SEQUENCE LISTING (10 sequences)

SEQ ID NO: 1 - protein, 8 aa, Artificial Sequence
  SIY peptide sequence
SIYRYYGL

SEQ ID NO: 2 - protein, 8 aa, Artificial Sequence
  SII peptide sequence
SIINFEKL

SEQ ID NO: 3 - protein, 9 aa, Artificial Sequence
  ROP7 peptide sequence
IPAAAGRFF

SEQ ID NO: 4 - protein, 9 aa, Artificial Sequence
  QL9 peptide sequence
QLSPFPFDL

SEQ ID NO: 5 - protein, 9 aa, Artificial Sequence
  GRA4 peptide sequence
SPMNGGYYM

SEQ ID NO: 6 - protein, 9 aa, Artificial Sequence
  ROP7 peptide sequence
IPAAAGRFF

SEQ ID NO: 7 - protein, 20 aa, Artificial Sequence
  p11b epitope
EWYDQSGLSVVMPVGGQSSF

SEQ ID NO: 8 - protein, 20 aa, Artificial Sequence
  p15c epitope
TFLTSELPGWLQANRHVKPT

SEQ ID NO: 9 - protein, 20 aa, Artificial Sequence
  p24c epitope
QRNDPLLNVGKLIANNTRVW

SEQ ID NO: 10 - protein, 20 aa, Artificial Sequence
  p27c epitope
LGGNNLPAKFLEGFVRTSNI

(19) United States
(12) Patent Application Publication
(10) Pub. No.: US 2011/0302665 A1
(43) Pub. Date: Dec. 8, 2011

(54) NON-HUMAN MAMMALS WITH T OR B CELLS HAVING PREDEFINED SPECIFICITY

(76) Inventors: Oktay Kirak, Boston, MA (US); Gijsbert M. Grotenbreg, Singapore (SG); Eva-Maria Frickel, London (GB); Rudolf Jaenisch, Brookline, MA (US); Hidde Ploegh, Brookline, MA (US)

(21) Appl. No.: 13/002,334

(22) PCT Filed: Jul. 2, 2009
(86) PCT No.: PCT/US09/49596

§ 371 Date: Aug. 24, 2011

Related U.S. Application Data
(60) Provisional application No. 61/077,835, filed on Jul. 2, 2008.
(60) Provisional application No. 61/077,807, filed on Jul. 2, 2008.

(51) Int. Cl. A01K 67/027; C12N 5/0781; C12N 5/071; C12Q 1/68; A61K 49/00; C12N 5/0735; C12N 5/0783; G01N 33/566

(52) U.S. Cl. 800/8; 435/325; 435/7.24; 435/6.12; 424/9.1

(57) ABSTRACT

The present invention provides non-human mammals, e.g., mice, generated from a T cell or B cell with a predefined specificity or isolated from an organism suffering from a condition of interest. In some embodiments the non-human mammals are not genetically modified. Also provided are methods of using the non-human animals.

### CROSS-REFERENCE TO RELATED APPLICATIONS

This application claims priority to, and the benefit of, U.S. Provisional Application No. 61/077,807, filed Jul. 2, 2008, and U.S. Provisional Application No. 61/077,835, filed Jul. 2, 2008. The entire contents of the afore-mentioned applications are incorporated herein by reference.

### GOVERNMENTAL FUNDING

The invention described herein was supported, in whole or in part, by Grants R37-CA84198 and RO1-HD045022 from the National Institutes of Health. The United States government has certain rights in the invention.

### BACKGROUND OF THE INVENTION

A properly functioning immune system plays important roles in defense against deleterious infectious agents ranging from viruses to multicellular parasites and in tumor immunosurveillance. It detects a wide variety of deleterious agents and contributes to their elimination. However, inappropriate or excessive immune system activation and/or recognition of self antigens by immune system effector cells can lead to adverse consequences such as autoimmune disease. The vertebrate immune system encompasses a variety of signaling molecules, proteins, cell types, organs, and tissues. Among these, T and B cells play a critical role in the adaptive immune response. A deeper understanding of the development and function of T and B cells would be of great scientific and medical significance.

### SUMMARY OF THE INVENTION

The present invention relates to methods of generating non-human mammals having T or B cells with predefined specificity. The invention also relates to uses of such non-human mammals and cells, e.g., for studying the immune system, for evaluating drug candidates and vaccines, etc.

Lymphocytes such as T and B cells generate their receptor repertoire by genetic rearrangement of their V(D)J regions. The combination of these V(D)J regions determines the specificity of the receptor. Using, for example, peptide-MHC complexes or antigens, T and B cells can be sorted according to their specificity. Such T and B cells with pre-defined specificity can then be used as donors for somatic cell nuclear transfer (SCNT) (or other methods to reprogram cells). The resulting embryonic stem (ES) cells as well as non-human mammals, e.g., mice, will carry the exact same rearrangement and therefore comprise cells having the exact same specificity. Without wishing to be bound by theory, an advantage of SCNT is that no genetic modification is introduced into the genome and the expression and regulation of those T and B cell receptors should thus be expected to mimic physiological levels. Similarly, somatic cell reprogramming would typically leave the T and B cell receptors under control of endogenous regulatory elements.

The procedure can also be used in a reverse approach in which lymphocytes are first reprogrammed (e.g., using SCNT or somatic cell reprogramming) and then ES cells and mice are generated. The specificity and immunological importance of such lymphocytes can then be characterized in those cells and animals. This is of interest in diseases such as cancer and autoimmunity, e.g., to assess the role of a certain lymphocyte subtype.

The mice of the instant invention that are generated via somatic cell nuclear transfer or somatic cell reprogramming of T cells or B cells are referred to herein as transnuclear (TN) mice or monoclonal mice.

In one aspect, the invention provides a method of producing a non-human mammal, the method comprising: (a) providing a non-human mammalian T or B cell that has a predefined specificity, wherein the mammalian T or B cell is not a natural killer (NK) T cell; and (b) generating a non-human mammal using the mammalian T or B cell, wherein at least some cells of the non-human mammal contain at least one TCR or BCR gene derived from the mammalian T or B cell. In some embodiments the mammalian T or B cell has rearranged TCR alpha and beta chain genes, or rearranged BCR heavy and light chain genes, respectively, and at least some cells of the non-human mammal contain rearranged TCR alpha and beta chain genes or BCR heavy and light chain genes, respectively, that assemble to form a TCR or BCR with the same specificity as those of the T or B cell. In some embodiments the cell is a conventional T cell. In some embodiments the cell is a CD8+ T cell. In some embodiments the T or B cell is specific for a predefined epitope. In some embodiments the predefined epitope is a peptide. In some embodiments the T or B cell is specific for a predefined antigen. In some embodiments the predefined antigen is a protein. In some embodiments the predefined antigen is produced by a microorganism. In some embodiments the predefined antigen is produced by a pathogen. In some embodiments the predefined antigen is a tumor antigen. In some embodiments the non-human mammal is a mouse. In some embodiments the T cell has a non-invariant TCR alpha chain. In some embodiments, generating the non-human mammal comprises reprogramming the nucleus of the T or B cell to pluripotency. In some embodiments, generating the non-human mammal comprises performing somatic cell nuclear transfer (SCNT) using a T or B cell with a predefined specificity as a nuclear donor. In some embodiments the SCNT is performed within 24 hours of isolating the T or B cell from an animal. In some embodiments the SCNT embryo is cultured in medium containing an inhibitor of histone deacetylase. In some embodiments the inhibitor is trichostatin A. In some embodiments, generating the non-human mammal comprises performing two step cloning. In some embodiments, said two step cloning comprises introducing ES cells into mouse tetraploid blastocysts by injection under conditions that result in production of an embryo. In some embodiments, the non-human mammal of the invention is not genetically modified. In some embodiments T and B cells of the non-human mammal do not contain a TCR or BCR transgene.

In some embodiments, the method of producing a non-human mammal comprises: (a) reprogramming a T or B cell that has a predefined specificity of interest to form an induced pluripotent stem (iPS) cell; and (b) generating a non-human mammal from the iPS cell. In some embodiments, the method of producing a non-human mammal comprises: (a) isolating from a first non-human mammal a T or B cell that has a predefined specificity of interest; and (b) generating a second non-human mammal from the T or B cell.

In some embodiments of the methods of producing a non-human mammal, the method comprises immunizing a first non-human mammal with an antigen of interest prior to isolating T or B cell(s) from the mammal, wherein an isolated T or B cell is used to produce a non-human mammal. In some embodiments the method comprises contacting the first non-human mammal with a microorganism or multicellular parasite of interest prior to isolating the T or B cell. In some embodiments the microorganism or multicellular parasite establishes an infection, e.g., survives and, in some embodiments, replicates, within the animal. In some embodiments, the step of isolating comprises: (a) obtaining T cells from the first non-human mammal; (b) contacting the T cells with an MHC-epitope complex; and (c) isolating a T cell that binds to the MHC-epitope complex. In some embodiments, the step of isolating comprises: (a) obtaining B cells from the first non-human mammal; (b) contacting the B cells with an epitope or antigen; and (c) isolating a B cell that binds to the epitope or antigen. In some embodiments, the step of isolating comprises: (a) obtaining B cells from the first non-human mammal; (b) culturing individual B cells under conditions in which antibody is secreted; and (c) isolating a B cell that secretes an antibody having the predefined specificity.

In some embodiments, the method further comprises isolating T or B cells from the non-human mammal generated from a T or B cell having predefined specificity or generated from a T or B cell obtained from an individual (e.g., non-human animal) having a disease or condition of interest (e.g., an infection, cancer, autoimmune disease). In some embodiments the method further comprising analyzing the T or B cells isolated from the generated non-human animal. In some embodiments the method further comprises analyzing the immune response of the generated non-human mammal to an antigen towards which the T or B cell has specificity.

In another aspect the invention provides a non-human animal, e.g., non-human mammal, produced according to a method described herein or descended from such an animal.

In some aspects, the invention provides method of producing a non-human mammalian ES cell, the method comprising: (a) providing a mammalian T or B cell that has a predefined specificity of interest, wherein the mammalian T or B cell is not a natural killer (NK) T cell; (b) introducing the nucleus of the T or B cell into an enucleated oocyte of the same species; (c) allowing the oocyte to develop into a blastocyst in vitro; and (d) isolating an ES cell from the blastocyst. The invention further provides an ES cell produced according to the foregoing method.

The invention provides an ES cell produced from a T or B cell with a predefined specificity. In some embodiments the T or B cell is a non-human cell. The invention provides an iPS cell produced from a T or B cell with a predefined specificity. In some embodiments the T or B cell is a non-human cell.

The invention further provides a non-human animal, e.g., non-human mammal, in which at least 50% of the T cells or at least 50% of the B cells are specific for a predefined antigen or epitope, and wherein T and B cells of the non-human animal do not comprise a TCR or BCR transgene, respectively. In some embodiments the mammal is a mouse. In some embodiments the non-human animal is not genetically modified.

The invention provides descendants of the non-human animals, which may be obtained by interbreeding, back-crossing, outbreeding, or cloning the non-human animals. The invention provides cells obtained from the non-human animals of the invention, cell lines derived therefrom, and animals generated from the cells, e.g., by SCNT or somatic cell reprogramming.

The invention provides a method of producing a non-human mammal, the method comprising: (a) providing a T or B cell isolated from an individual suffering from or at risk of a disease; and (b) generating a non-human mammal from the T or B cell. In some embodiments the individual suffers from a tumor and the T or B cell is isolated from the tumor. In some embodiments the individual suffers from or is at risk of an autoimmune disease or infection and the T or B cell is isolated from tissue affected by the autoimmune disease or infection (e.g., tissue that has suffered damage or reduced function as a result of the condition). In other embodiments the T or B cell is isolated from tissue not apparently affected by the autoimmune disease or infection. In some embodiments the individual suffers from or is at risk of diabetes (e.g., Type I diabetes) and the T or B cell is isolated from the pancreas. In some embodiments the individual suffers from a tumor and the T or B cell is isolated from the tumor. The condition, e.g., infection or tumor, could be naturally occurring or could be experimentally induced. In some embodiments the individual has received a transplant of non-autologous tissue. In some embodiments the T or B cell is isolated from the transplanted tissue. The invention further provides a non-human animal, e.g., mammal, produced according to any of the methods.

Specific embodiments of the invention are described in more detail below. However, these are illustrative embodiments, and should not be construed as limiting in any respect. It is contemplated that embodiments described herein are applicable to various different aspects of the invention. It is also contemplated that any of the embodiments or aspects described herein can be freely combined with one or more other such embodiments or aspects whenever appropriate.

### BRIEF DESCRIPTION OF THE DRAWINGS

FIG. 1 shows a schematic diagram of a method for performing somatic cell nuclear transfer (SCNT). The upper panel illustrates one step, or “direct” cloning approach. In this approach, a somatic cell nucleus is isolated and introduced into an enucleated oocyte, which is allowed to begin developing, e.g., to a blastocyst. The blastocyst is transferred to a pseudopregnant female. Resulting offspring contain cells whose genetic material is derived from the somatic cell nucleus. The lower panel illustrates the two step, or “indirect” cloning approach. In this approach, a somatic cell nucleus is isolated and introduced into an enucleated oocyte, which is then introduced into a blastocyst. ES cells are isolated from the blastocyst and used to generate an animal. In both approaches, SCNT converts a somatic cell into a cell with an ESC-like state without modifying the sequence of its genome.

FIG. 2A shows a summary of steps involved in generating a mouse from a T or B cell with specificity for a predefined epitope using the indirect cloning approach, and approximate timeline. FIG. 2B shows another representative timeline with representative photographs illustrating the steps. −X indicates that the mouse is immunized with an antigen of interest or infected with a microorganism at a time X prior to harvesting cells. Harvested cells are analyzed, e.g., by staining and FACS sorting, to identify those specific for the epitope or antigen of interest.

FIG. 3. Design and application of conditional ligands for H-2L^{d} class I major histocompatibility complex (MHC) molecules. A, Rendering of the p29 epitope (orange) in complex with H-2L^{d} (omitted for clarity, PDB-ID 1LD9). Individual replacement of the P6 to P8 residues with the synthetic Anp-residue produced photocleavable derivatives p29-P6*, p29-P7, and p29-P8*, respectively. B, Class I MHC tetramers composed of H-2K^{b}/SV9-P7* [ref. 16 of Example 2] or H-2L^{d} preloaded with either p29-P6*, p29-P7*, or p29-P8* were UV-irradiated in the presence or absence of the peptides SIYRYYGL (SIY) (SEQ ID NO: 1), SIINFEKL (SII) (SEQ ID NO: 2), IPAAAGRFF (ROP7) (SEQ ID NO: 3), or QLSPFPFDL (QL9) (SEQ ID NO: 4) as indicated and used to stain OT-1 or 2C TCR transgenic cells. Functional staining reagents were obtained only in case of the correct pMHC-T cell receptor combinations. PE, phycoerythrin.

FIG. 4. Screening for endogenous Toxoplasma gondii-derived CD8⁺ T cell epitopes with caged major histocompatibility complex (MHC) tetramers. A, Schematic depiction of a T. gondii tachyzoite with the relevant organelles highlighted. B, Origin and numbers of proteins, as well as the candidate 9-mer epitopes embedded therein, that were selected for MHC tetramer screening. Redundancy in the selected proteome generates a surplus of 263 epitopes from 246 unique peptide sequences. C, Staining of CD8⁺ T cell-enriched splenocytes with H-2L^{d}/SPMNGGYYM (GRA4) (SEQ ID NO: 5) on day 14 after infection or H-2L^{d}/IPAAAGRFF (ROP7) (SEQ ID NO: 6) 42 days after infection reveals the antigen-specific CD8⁺ T cell subpopulations after infection with T. gondii. These reagents were obtained separately after photocleavage and peptide-exchange on the caged H-2L^{d}/p29-P8* tetramer complex. PE, phycoerythrin.

FIG. 5. Kinetic distinctions in cellular immune responses to the GRA4- and ROP7-derived epitopes. A, Cell surface staining with the appropriate class I major histocompatibility complex tetramer plotted as percentage of tetramer-positive cells among CD8⁺ cells versus the time point after infection shows maximal expansion of the GRA4-specific CD8⁺ lymphocyte population at ˜14 days. The population of CD8⁺ T cells that recognize the ROP7 epitope peak between 4-6 weeks after infection. These phenomena were generally comparable over the different tissues sampled (spleen, peritoneal cavity [PEC], and brain). Data shown are representative of >3 independent experiments. B, To compare CD69 staining, cells were gated on the CD8⁺ population (infection⁻) or the H-2L^{d}/GRA4-specific or H-2L^{d}/ROP-specific population (infection⁺; 2 and 4 weeks after infection, respectively). The percentage of cells that were CD69-high, as specified per plot, was increased for the antigen-specific population. Pregating on CD8⁺ splenocytes, both populations of H-2L^{d}/GRA4-specific T cells (2 weeks after infection) and H-2L^{d}/ROP7-specific T cells (4 weeks after infection) are high for the surface marker CD44. The percentage of antigen-specific cells is indicated in the lower quadrant. C, In vitro stimulation of splenocytes from T. gondii-infected BALB/c mice with the addition of either no peptide (−), enterotoxin (ET), SPMNGGYYM (GRA4) or IPAAAGRFF (ROP7) epitope, or the known H-2L^{d}-binding peptide QLSPFPFDL (QL9) demonstrated that both H-2L^{d}/GRA4 and H-2L^{d}/ROP7 CD8⁺ T cells produce interferon (IFN)-γ in an epitope-specific fashion. The gating for total CD8⁺ T cells excluded the tetramer-positive population. APC, allophycocyanin; FITC, fluorescein isothiocyanate; PE, phycoerythrin.

FIG. 6. Stage-specific delivery and expression of antigens and their effect on the specific CD8⁺ T cell repertoire. A, Toxoplasma gondii mutants defective in establishing a chronic infection elicit differential epitope-specific CD8⁺ T cell responses at 4 and 8 weeks after infection. For details, see Example 2(* P<0.05, by Welch's t test). B, Western blot analysis of the expression of ROP7 versus GRA4 in T. gondii ME49 tachyzoites and in vivo-generated bradyzoites shows GRA4 to be strongly expressed in the tachyzoite stage only, whereas ROP7 exhibits similar expression profiles in both parasitic stages. SAG2Y is exclusively expressed in bradyzoites, and α-tubulin was used as loading control. PE, phycoerythrin.

FIG. 7. Characterization of Pru mutant 76-E2. A, Mice infected with the 76-E2 mutant do not form large cysts (25-50 μm) during chronic infection. CBA/J mice (JAX labs) were injected intraperitoneally with 2×10⁴ tachyzoites. Four weeks after inoculation, mice were sacrificed, and the brains were stained with fluorescein isothiocyanate-conjugated Dolichos biflorus to visualize the cysts [ref. 21 of Example 2]. Images were taken on a Zeiss Axiovert 100TV with a 63× objective. The solid white line indicates 50 μm. B, the pLK47 insertion site for the 76-E2 mutant is in the 3′ end of the GRA3 locus on chromosome X at position 992,067 bp and is indicated by the vertical line. The diagram is adapted from the genome browser of ToxoDB (ToxoDB4.3 [ref 32 of Example 2]). Red arrow boxes, gene transcripts and their direction. Lines, predicted introns. C, Disruption of the GRA3 locus as seen by Northern blot analysis. Tachyzoite RNA from both the 76-E2 mutant and wild-type parasites was probed with the GRA3 ORF. An α-tubulin probe was used as a loading control. 3′ Rapid amplification of cDNA end by polymerase chain reaction indicated multiple polyadenylation sites at the GRA3 locus, some of which were disrupted by the insertion of the pLK47 plasmid in the 76-E2 mutant. These disrupted mRNAs were shifted up in size, most likely as a result of read-through into the pLK47 plasmid. D, Western blot analysis showed that the GRA3 protein is reduced but not eliminated in the 76-E2 mutant. Polyclonal antibodies were used to detect both GRA3 and β-tubulin proteins (courtesy of Keith Joiner and David Sibley, respectively) in wild type, host cell only (human foreskin fibroblasts [HFF]), and 76-E2. While the β-tubulin was designed to be T. gondii specific, it does cross-react to a minimal extent with HFF cells. E, Histone deacetylase 3 (HDAC3) does not appear to be disrupted in the 76-E2 mutant. Tachyzoite RNA from both the 76-E2 mutant and wild-type parasites was probed with the HDAC3 ORF. An α-tubulin probe was used as a loading control.

FIG. 8. Serum IgG reaction to Toxoplasma gondii in BALB/c mice infected with 2×10⁶ γ-irradiated Prugniaud tachyzoites. A, Western blot analysis. T. gondii tachyzoite lysate was incubated with the indicated BALB/c serum and the blot was probed with total mouse-IgG antibody. B, Human foreskin fibroblasts previously infected with T. gondii RH YFP2 [ref. 33 of Example 2] were fixed with formaldehyde, permeabilized with saponin, and incubated with a 1:100 dilution of the denoted BALB/c serum. Alexa-Fluor 647-conjugated mouse IgG antibody (Molecular Probes) was used to probe for serum antibodies against T. gondii.

FIG. 9. Agreement among the predicted GRA4 protein sequences. Alignment of 2 current protein prediction algorithms available on ToxoDB [ref. 32 of Example 2]; red box, sequence of the GRA4 epitope. Note that only the TwinScan model contains the exon encompassing the region that encodes for the epitope.

FIGS. 10A and 10B show an overview of the MHC tetramer strategy.

FIG. 11 shows FACS analysis of T cells from T. gondii-infected Balb/c mice, illustrating the presence of T cells specific for epitopes from Gra4 or Rop7.

FIG. 12 shows results of SCNT and ES cell line derivation using T cells specific for T. gondii epitopes as donors. The T cells were obtained from Balb/C mice.

FIG. 13 shows results of SCNT and ES cell line derivation using T cells specific for T. gondii epitopes as donors. The T cells were obtained from Balb/C×BL/6 F1 mice.

FIG. 14 shows generation of chimeric mice following SCNT. The left panel shows chimeric mice with contribution from T cells obtained from Balb/C×BL/6 F1 mice.

FIG. 15 shows results of FACS analysis of T cells obtained from chimeric mice and stained with Ld-Rop7, Ld-Gra4, or Kb-A4 tetramers, as indicated. The results show that the chimeric mice contain CD8+ cells specific for the T. gondii epitopes Rop7, Gra4, or Kb-A4. FIG. 15A: Top panels show representative flow cytometry plots of BL6×Balb/c F1 background (left) or Balb/c (right) mice infected with T. gondii. Gate and number (% per total CD8+ T cells) indicates sorted specific CD8+ T cells with defined specificity. Lower panels show representative flow cytometry analysis of chimeric mice injected with SCNT-ES cells derived from according donor cells in top panel. Box and number (% per total CD8+ T cells) indicates the presence of naive specific CD8+ T cells in chimeric mice. FIG. 15B shows the same results shown in FIG. 15A with the panels organized differently and some additional results of similar experiments.

FIG. 16A shows offspring resulting when chimeric mice derived from T cells obtained from BL/6×Balb/c F1 mice were back-crossed into BL/6. Germline transmission can be evaluated by agouti coat color (black arrow). FIG. 16B shows offspring resulting when chimeric mice derived from T cells obtained from BL/6×Balb/c F1 mice were back-crossed into Balb/c, demonstrating contribution to the germline. Germline transmission can be evaluated by albino coat color.

FIG. 17A shows results of FACS analysis of T cells obtained from chimeric mice arising from SNCT using T cells specific Rop7 or Gra4. The T cells were stained with Ld-Rop7 or Ld-Gra4 tetramers as indicated, and demonstrate that the chimeric mice contain CD8+ T cells specific for the T. gondii epitopes Rop7 or Gra4. FIG. 17B shows PCR amplification of genomic DNA (B) on wildtype (W) and offspring (G) carrying the specific TCR rearrangements. Specific PCRs were set up to detect either the rearranged (*) only (alpha-chain of Kb-Tg-tgd05759-66) or both the wildtype (−) and according rearranged (*) alpha- and beta-chains. FIG. 17C is a table summarizing the breeding and germline transmission of chimeric transnuclear mice. FIG. 17D shows additional flow cytometric analysis of peripheral blood from various TN mice.

FIG. 18A shows TCR sequence and comparison of transnuclear T cells with wildtype and transgenic mice. Schematic representation of the TCR-alpha (top panel) and TCR-beta locus (lower panel) of rearrangements in transnuclear mice. Top row represents wildtype configuration and some regions for orientation (data based on www.ensembl.org).

FIG. 18B shows a summary of results achieved using two different mouse strains and three different epitopes. A total of 7 ES cell lines were derived from T cells specific for Rop7, Gra4, or A4. Three of these lines contributed to the germline in chimeras, resulting in mice having T cells specific for Rop7 (two lines) or Gra4 (one line).

FIG. 19A-19F shows DNA and protein sequence of TCRs. Sequence from the start codon to the beginning of constant region for the alpha and beta chains of the TN line Kb-Tg-tgd05759-66 (A), Ld-Tg-Gra4107-115 (B), Ld-Tg-Rop7161-169-I (C), Ld-Tg-Rop7161-169-II (D), and Ld-Tg-Rop7161-169-III (E). Amino acid sequence of the CDR3-regions of transnuclear mice (F).

FIG. 20. Flow cytometric analysis of spleen from wildtype, transnuclear and transgenic mice. Splenocytes were compared first for their CD3+ and B220+ population (upper row). The CD3+ population was then analyzed for their CD4 and CD8 expression (middle row), and the CD8+ population was analyzed for their expression of CD44 and CD62L (lower row).

FIG. 21. Development of T cells in transnuclear versus transgenic mice. Flow cytometric analysis of thymus from wildtype, transnuclear and transgenic mice (A). Thymocytes were first gated on γδ⁻, NK1.1⁻, CD19⁻ cells and then analyzed first for their CD4 and CD8 expression (upper row). CD4-CD8 double-negative cells were then analyzed for their CD44 and CD25 expression (lower row). The CD4-CD8 double-negative and CD8 single-positive population was analyzed for their expression of CD69 and CD5 (B).

FIG. 22. Functionality of transnuclear T cells and germline transmission. Dissociation of peptide-L^{d} tetramers from CD8⁺ T cells was measured for the transgenic line 2C and the transnuclear lines L^{d}-Tg-Rop7¹⁶¹⁻¹⁶⁹ and L^{d}-Tg-Gra4¹⁰⁷⁻¹¹⁵ using FACS and plotted over time (A). Dissociation of the peptide-K^{b} tetramers from CD8⁺ T cells for the transgenic line OT-I and the transnuclear line K^{b}-Tg-tgd057⁵⁹⁻⁶⁶ (B). Stabilization of H-2L^{d} on the surface of TAP−/− cells via titration of QL9, Rop7¹⁶¹⁻¹⁶⁹ or Gra4¹⁰⁷⁻¹¹⁵ peptide (C) (analyzed using Graphpad Prism software). Flow cytometry analysis of IFN-γ secretion (D) upon stimulation of transnuclear T cells with antigen-presenting cells loaded with control peptide (upper panel) or specific peptide (lower panel). (E) Purity of T cells after negative selection for CD8 (upper left blot) and their expression of CD69 (lower left blot). Dilution of CFSE and upregulation of CD69 upon in vivo challenge with T. gondii (middle and right column). Survival curve of wildtype B6CF1 mice infected with lethal dose of tachyzoites (n=3 for each group) (F). Flow cytometric analysis of the presence of tgd057⁵⁹⁻⁶⁶ specific T cells in mice expressing various combination of the α- or β-chain of the given TCR (G).

FIG. 23. Flow cytometric analysis of blood cells from a transnuclear B cell-derived mouse expressing Ovalbumin-specific IgG1. Control mouse (left) with 17.4% IgG1⁺ B cells and almost no specificity for Ovalbumin (0.17% and 0.19%). TN IgG1-Ova mouse has almost exclusively IgG1⁺ B cells (2.59%+97.4%) with the great majority being specific for Ovalbumin (97.4%).

FIG. 24. ELISA analysis of serum immunoglobulins from transnuclear mice derived from B cells expressing Ovalbumin-specific IgG1.

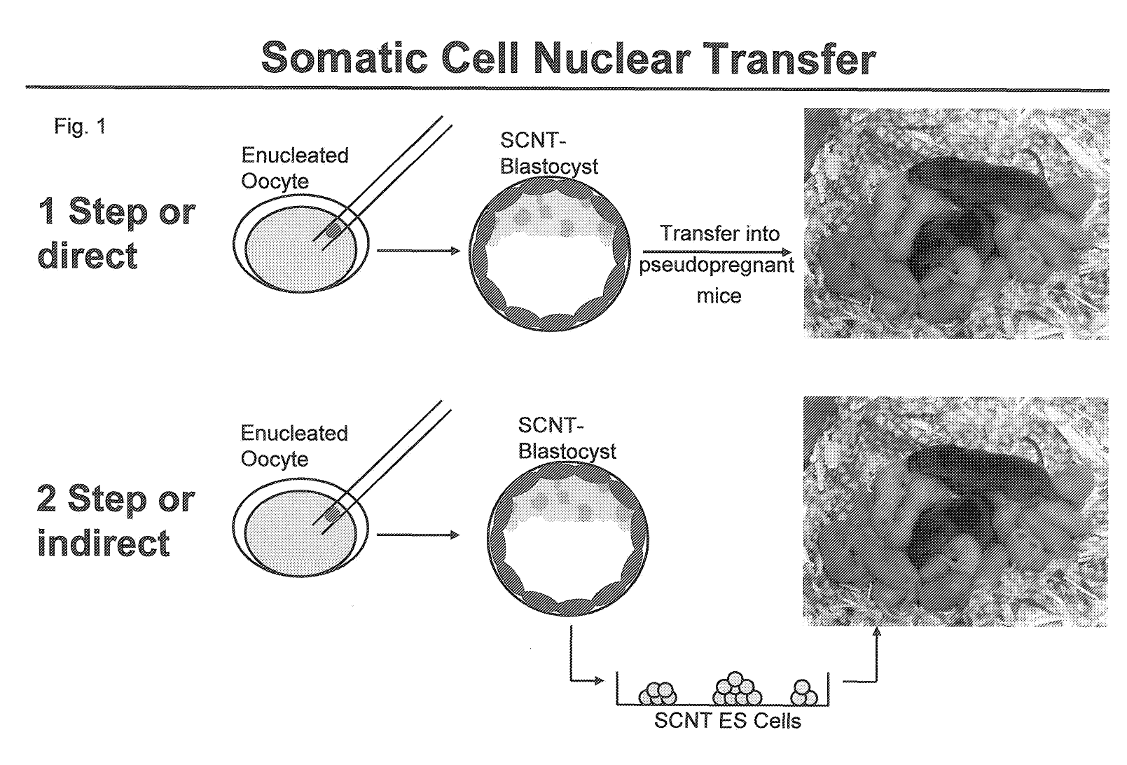

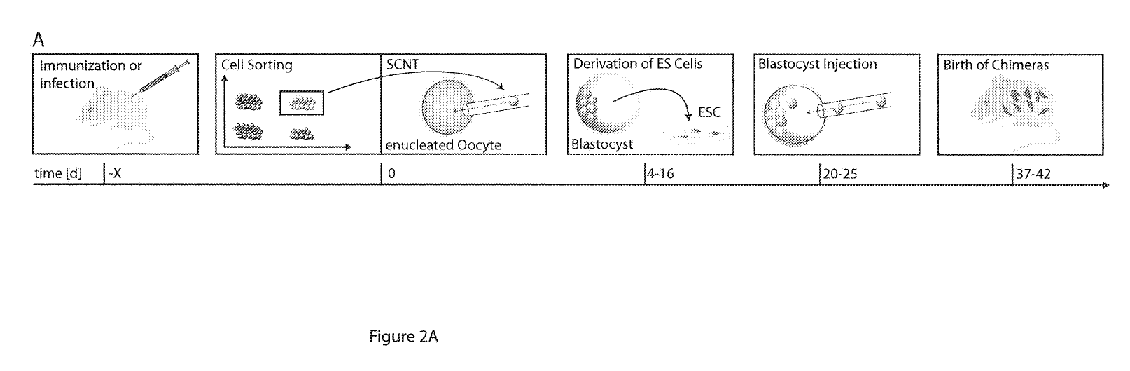

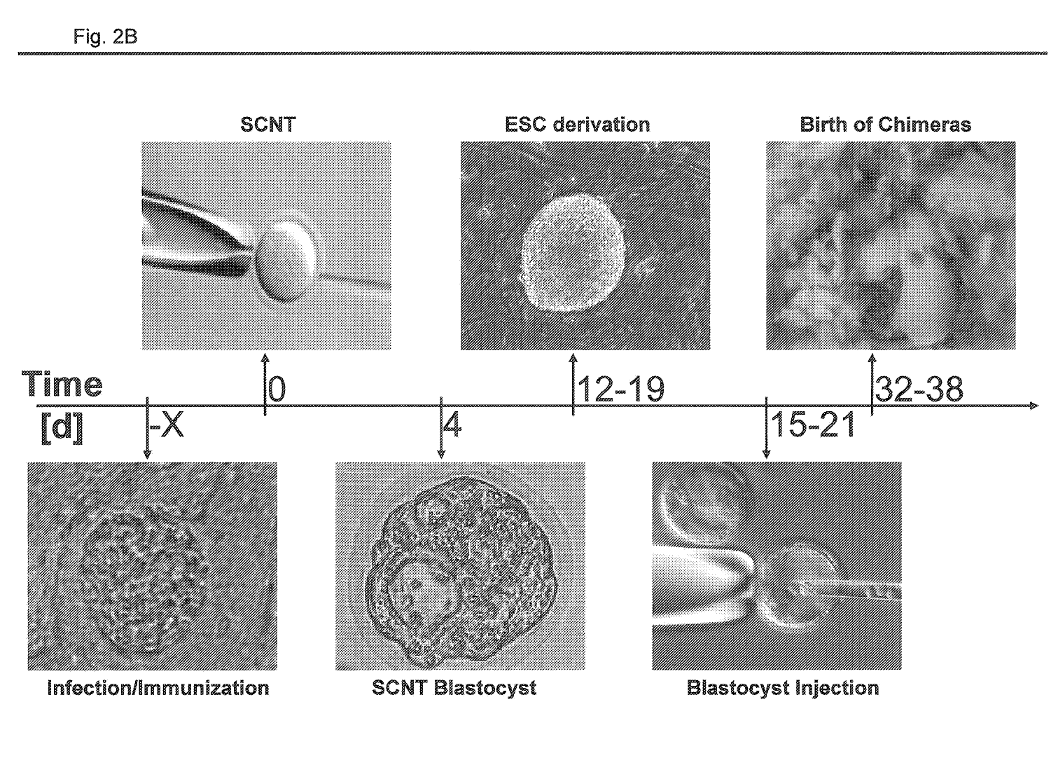

### DETAILED DESCRIPTION OF THE INVENTION

Mature T and B cells display T cell receptors (TCR) and B cell receptors (BCR), respectively, on their surface, which are responsible for recognizing and binding to particular epitopes and/or antigens with a certain, in some instances high, affinity and specificity. Applicants have developed novel methods to generate non-human mammals with T or B cells that have specificity for a predefined epitope or antigen of interest. Certain of the methods involve providing a non-human mammalian T or B cell that has a predefined specificity and generating a non-human mammal using the mammalian T or B cell, wherein at least some cells of the non-human mammal contain TCR or BCR genes derived from the non-human mammalian T or B cell. Other methods of the invention involve isolating a T or B cell from a non-human mammal suffering from or at risk of a condition of interest and generating a non-human mammal from the T or B cell. The condition of interest is, in at least some embodiments, one in which the immune system contributes to the condition or to defense against the condition. For example, the condition may be an autoimmune disease or a tumor or an allergic condition. The T or B cell may be isolated from a tumor or from an organ or tissue that is subject to T or B cell attack in an autoimmune condition. The non-human mice of the instant invention that are generated via somatic cell nuclear transfer of T cells or B cells with pre-defined specificity, or that are generated by somatic cell reprogramming of T cells or B cells with pre-defined specificity, represent a new type of mouse model, and are referred to herein as transnuclear (TN) mice or monoclonal mice. The non-human mice of the instant invention that are generated by SCNT of T cells or B cells isolated from a mouse suffering from or at risk of a condition of interest, or that are generated by somatic cell reprogramming of T cells or B cells isolated from a mouse suffering from or at risk of a condition of interest, are also referred to herein as TN mice or monoclonal mice. The present invention may find particular use as applied to mice. However, the invention is not limited to mice but may be applied to other non-human animals, e.g., non-human mammals or avians. For example, various embodiments of the invention relate to murine, caprine, ovine, bovine, porcine, canine, feline and non-human primate species, e.g., cows, pigs, horses, sheep, rabbits, guinea pigs, monkeys, rats, etc. For purposes of description the instant specification mainly refers to mice, but it should be understood that the invention provides embodiments relating to other non-human animals.

A T or B cell to be used in certain of the inventive methods is specific for a predefined epitope or antigen. The term “predefined” is used to mean that information regarding the identity of the epitope or antigen for which the T or B cell is specific is known or readily available prior to using the T or B cell in the inventive method. “Identity” could be expressed in terms of sequence (e.g., in the case of epitopes or antigens that are polypeptides or nucleic acids, or portions of either), chemical formula, structure, or any combination of functional and/or structural identifying characteristics or instructions regarding how to make or obtain the epitope or antigen. In certain embodiments of the present invention, a T or B cell is considered specific for a predefined epitope or antigen (also referred to as epitope or antigen of interest), if it has been subjected to a sorting or selection process that separates T or B cells that bind to particular epitope or antigen of interest from other T or B cells that have significantly lower (e.g., on average at least 10-fold lower, or at least 100-fold lower) binding affinity for the epitope or antigen of interest.

Most T cells express a TCR composed of α and β chains, which assemble to form an αβ dimer. γδ T cells are a minority population and possess an alternative TCR composed of γ and δ chains. The TCR and BCR are generated through processes that collectively generate the immense diversity of binding specificities represented in a typical vertebrate immune system. Briefly, each TCR or BCR chain contains a variable (V) region and a constant (C) region. The variable region, which contains the portion of the chain that contributes to the epitope binding site, is composed several distinct gene segments (e.g., V, D, and J in the case of the TCR β chain or BCR heavy chain; V and J in the case of the TCR α chain or BCR light chain). These segments are rearranged and brought into proximity with one another to generate a complete coding sequence in a sequential process. There are multiple copies of the V and J segments for each chain in germline DNA, with differing sequences. Many different V regions can thus be generated by selecting different combinations of these segments. Utilization of one gene segment of each type makes possible the great diversity of variable regions found among the TCRs and BCRs expressed by T and B cells in a given individual. Similar mechanisms give rise to antibody molecules, which resemble BCR but contain a domain responsible for directing secretion of the molecule rather than a domain that spans the plasma membrane. Recombination activating gene 1 (RAG 1) and RAG2 are proteins that mediate V(D)J recombination in developing T and B cells, which allows for production of TCR, BRC, and antibodies. It will be appreciated that additional mechanisms such as somatic hypermutation, gene conversion, etc., may also contribute to immunoglobulin gene diversification. See, e.g., Janeway's Immunobiology, 7^{th} ed. Murphy, K., et al, Garland Science Taylor & Francis Group (2008), which provides extensive information on these and other aspects of the immune system. See also Chaudhuri, J. and Alt, F., Nature Reviews Immunology, 4: 541-552 (2004). As used herein, a “conventional” T cell is one whose TCR recognizes an epitope in complex with an MHC molecule and/or whose TCR has undergone V(D)J rearrangement without being restricted to utilization of particular V or J gene segments. There also exist certain “unconventional” T cell subsets bearing semi-invariant or invariant TCRs (e.g., TCRs whose α or β chain contains specific V and/or J gene segments), such as CD1d-restricted Natural Killer (NK) T cells. These T cells are distinct in a number of ways from the conventional T cells whose TCR arises from typically unrestricted V(D)J recombination.

Consistent with usage in the art, a T or B cell is said to “be specific for” or “have specificity for” or a predefined epitope or antigen, if the cell expresses a TCR, BCR, or antibody that recognizes and binds to the epitope or antigen with significantly higher affinity and/or specificity relative to interactions that the TCR, BCR, or antibody may have with most other epitopes or antigens. It will be appreciated that T cells recognize epitopes that are bound to a major histocompatibility complex (MHC) molecule. T cells that express the CD8 co-receptor (CD8+ T cells) recognize epitopes bound to Class I MHC molecules, while T cells that express the CD4+ co-receptor (CD4+ T cells) recognize epitopes bound to Class II MHC molecules. In contrast, the BCR and antibody molecules can recognize epitopes in solution or on the surface of cells or pathogens present as antigens, without the need for processing and MHC presentation. The situation in vitro might vary for TCRs, BCRs as well as antibodies such that their epitopes or antigens might be recognized in a different context, e.g. antibodies can detect their antigen also when bound to a membrane used for example in Western blots.

It is understood that a TCR that is specific for an epitope binds to the epitope when the epitope is properly complexed with an appropriate MHC molecule, and a TCR that is specific for an antigen binds to an epitope contained within the antigen, e.g., an epitope that results from processing of the antigen (e.g., by an antigen presenting cell), when the epitope is properly complexed with an appropriate MHC molecule. In exemplary embodiments, a TCR or BCR is specific for an epitope or antigen if cells that express the TCR or BCR can be readily separated from cells that do not express the TCR or BCR (e.g., cells that express different TCR or BCR or that do not express any TCR or BCR) by contacting the cells with the epitope or antigen and a label, e.g., a fluorescent label, maintaining the cells under conditions suitable for TCR or BCR binding to an epitope, and subjecting the cells to fluorescence activated cell sorting (FACS). It will be appreciated that in the case of the TCR, the epitope should be complexed with an appropriate MHC molecule, as discussed further elsewhere herein. The epitope, epitope/MHC complex or antigen may be directly or indirectly linked to the fluorescent label. Alternately, the epitope, epitope/MHC complex, or antigen is not labeled, but after allowing binding to occur, the cells are contacted with a labeled antibody or other binding agent that binds to the epitope/MHC complex or antigen, and the cells are subjected to FACS after allowing binding to occur. One of skill in the art will be able to select FACS parameters that distinguish between cells that have specifically bound the epitope or antigen and those that display a background level of (nonspecific) binding. See, e.g., Example 1 and 2 and references therein. It will be appreciated that a variety of methods may be used to determine that a TCR, BCR, or antibody is specific for an epitope or antigen of interest, and a variety of methods can be used to enrich for specific T or B cells, such as MACS® (see further discussion below).

In certain of the inventive methods, a T or B cell specific for a predefined epitope or antigen is used as a donor of a genome (e.g., a donor of a nucleus comprising a genome) that includes rearranged TCR or BCR genes. For example, the T or B cell has rearranged TCR α and β chain genes or rearranged BCR heavy and light chain genes, respectively. It is also within the scope of certain embodiments of the invention to use a T cell or B cell that has rearranged only a single chain of its receptor. In most embodiments of the invention, a T cell does not comprise an invariant alpha chain. Using any of a variety of methods, the genome is reprogrammed to a pluripotent state, and the reprogrammed pluripotent genome is used to generate a cloned non-human mammal or a chimeric non-human mammal containing at least some descendant cells whose genome is derived from the genome of the original T or B cell, i.e., the genome of the descendant cells resulted from successive cell divisions in which the genome of the original T or B cell was copied by DNA synthesis and transmitted to daughter cells. The genome of such cells may be essentially identical in genetic sequence to that of the donor T or B cell. The term “essentially identical” is used to take into account the fact that uncorrected errors in DNA copying may occur at a low frequency (e.g., less than about 1 in 10⁵ base pairs) and may be transmitted to daughter cells, resulting in slight variations in sequence.

The Applicants hypothesized that descendant cells should thus contain the same TCR or BCR gene segment rearrangements as found in the T or B cell from which they were derived. The Applicants further hypothesized that at least some T or B cells in the cloned or chimeric animal would express the rearranged TCR or BCR, respectively, resulting in a non-human mammal having T cells or B cells with specificity for the predefined epitope or antigen. As described in Example 3, the Applicants used CD8+ T cells with predefined specificity for epitopes found in T. gondii proteins to generate mice and demonstrated that these mice indeed contain CD8+ T cells specific for these epitopes in the absence of immunization. The results show that the rearranged TCR α and β chains are expressed and form functional TCRs in T cells of mice whose cells all contain the correct, i.e., rearranged, TCR α and β genes inherited from the original T cell. It is believed that these results represent the first instance of sorting conventional T cells to select those specific for a predefined antigen and then using the selected T cells to generate an ES cell. It is also believed that these results represent the first instance of sorting conventional T cells to select those specific for a predefined antigen and then using the selected T cells to generate a mouse.

A T or B cell to be used in the inventive methods may be specific for any of a wide variety of epitopes or antigens. Epitopes of interest include essentially any molecular structure that can be recognized and specifically bound by a TCR or BCR generated by rearrangement of the TCR or BCR locus of an animal, e.g., a mammal, e.g., a non-human mammal, e.g., a mouse. As noted above T cells recognize an epitope presented in complex with MHC Class I or II molecules. Epitopes of interest may be portions of a nucleic acid or protein expressed by an organism, e.g., a microorganism (which term is used herein to encompass viruses, bacteria, fungi, and protozoa) or a multicellular parasite (e.g., a helminth, arthropod). Viruses of interest include, e.g., single or double stranded DNA or RNV viruses, retroviruses, etc. They may belong, e.g., to the following families: Adenoviridae, Picornaviridae, Herpesviridae, Hepadnaviridae , Flaviviridae, Retroviridae, Orthomyxoviridae, Paramyxoviridae, Papovaviridae, Rhabdoviridae, Reoviridae, Togaviridae. Specific examples are HBV, HCV, HIV, EBV, CMV, measles, influenza virus. Bacteria of interest include, e.g., gram positive bacteria, gram negative bacteria, acid fast bacteria, etc. Examples are Mycobacteria, e.g, M. tuberculosis, Chlamydia, e.g., C. trachomatis, Staphylococcus, Streptococcus, Pseudomonas, Enterococci, Enterobacteriaceae (Klebsiella, Salmonella, Serratia, Yersinia), Erysipelothrix, Helicobacter, Legionella, Leptospires, Listeria, Mycoplasmatales, Neisseriaceae (e.g., Acinetobacter, Menigococci), Pasteurellacea (e.g., Actinobacillus, Heamophilus, Pasteurella), Rickettsia, Bacillaceae (e.g., Anthrax, Clostridium), Bacteroidaceae, etc. Fungi of interest include Cryptococcus, Coccidia, Histoplasma, Candida, Aspergillus, Blastomyces, etc. Parasites of interest include, e.g., Apicomplexans such as Toxoplasma, Cryptosporidium, or Plasmodium; kinetoplastids such as Trypanosomes; worms, e.g., nematodes such as Ascaris, cestodes, trematodes (often called flukes), etc. In some embodiments the organism is a pathogen, i.e., an organism responsible for disease. In some embodiments the organism is one that establishes a latent or chronic infection in at least some hosts. In some embodiments the organism is an intracellular pathogen, i.e., it replicates intracellularly and/or resides intracellularly during at least part of its life or during one or more stages of its life cycle. In some embodiments the epitope is one that is associated with allergy and/or type I hypersensitivity reactions.

In some embodiments the epitope is a linear epitope. In some embodiments an epitope is a conformational epitope. In some embodiments the epitope is a continuous epitope. In some embodiments the epitope is a discontinuous epitope. One of skill in the art will recognize that not all peptides or molecular structures are capable of serving as epitopes. A number of T or B cell epitopes are known in the art. See, e.g., The Immune Epitope Database and Analysis Resource (IEDB) (http://www.immuneepitope.org/home.do). Computational epitope prediction tools are available.

Antigens of interest include essentially any molecular structure that contains at least one epitope. In some embodiments the antigen of interest is a molecule expressed by an organism, e.g., a microorganism or multicellular parasite. In some embodiments the antigen is one that has been associated with allergy and/or type I hypersensitivity reactions. Such antigens may be found, e.g., in foods, drugs, house dust, animal dander or fur, plants (e.g., pollen), etc.

In some embodiments, the antigen of interest is a tumor antigen. Tumor antigens can be any molecule or component thereof that is expressed or present selectively or exclusively in or on the surface of tumor cells relative to other cells in the body of the subject in which the tumor occurs. Tumor antigens are typically not present in individuals not suffering from a tumor, or may be expressed at lower levels or in a different context or otherwise aberrantly in an individual suffering from a tumor, hence the immune system may recognize them as “non-self” and mount a response against them. Mutation of proto-oncogenes and tumor suppressor genes can lead to production of abnormal proteins, which may act as tumor-specific or tumor-associated antigens (collectively referred to herein as tumor antigens). Proteins that are normally produced in very low quantities but whose production is significantly or dramatically increased in tumor cells are of interest. Oncofetal antigens are another important category of tumor antigen. Examples are alphafetoprotein and carcinoembryonic antigen, proteins that are normally produced in the early stages of embryonic development and disappear by the time the immune system is fully developed. Other tumor antigens are: CA-125 (associated, e.g., with ovarian cancer), MUC-1, epithelial tumor antigen, and melanoma-associated antigen (e.g., melanoma-associated antigen 3). Abnormal proteins may also produced by cells infected with oncoviruses, e.g., Epstein-Barr virus (EBV); papilloma virus, e.g., human papilloma virus (HPV) such as HPV6, 11, 16 or 18; herpes virus, e.g., human herpes virus 8; hepatitis virus, e.g., hepatitis B or C virus; polyoma virus such as Merkel cell polyoma virus. Substances such as cell surface glycolipids and glycoproteins may also have an abnormal structure in tumor cells and could contain epitopes of interest. In some embodiments, the tumor antigen is a telomerase reverse transcriptase or portion thereof, e.g., human telomerase reverse transcriptase (hTERT) or a peptide derived therefrom such as I540, 572Y, or 988Y (Vonderheide, V R, Biochimie, 90: 173-180 (2008)). In some embodiments a tumor antigen is associated with a carcinoma. In some embodiments a tumor antigen is associated with a sarcoma. In some embodiments a tumor antigen is associated with a hematologic malignancy, e.g., a lymphoma or leukemia or myeloma. In some embodiments a tumor antigen is associated with breast cancer (e.g., a breast cancer antigen such as EGFR (epidermal growth factor receptor) or I IFR2 antigen), bladder, bone, brain, cervical, colon, endometrial, esophageal, head and neck, laryngeal, liver, lung (small cell or non-small cell), ovarian, pancreatic, prostate, stomach, renal, skin (e.g., basal cell, melanoma, squamous cell), testicular, or thyroid cancer.

It should be noted that the particular epitope(s) within an antigen of interest that are recognized by T or B cells may or may not be known initially. Furthermore, in some instances it is unknown which proteins or other molecules expressed by a microorganism, tumor, parasite, etc., are antigenic. In some embodiments of the invention, an epitope recognized by T cells is identified. In some embodiments of the invention, an epitope recognized by B cells is identified.

A T or B cell for use in the present invention is one that has undergone rearrangement of its TCR or BCR, resulting in a cell that is specific for a predefined epitope or antigen. The T cell may be a CD4+ cell, a CD8+ cell, etc. In general, both chains (e.g., the α and β chains) of the TCR or BCR will have undergone rearrangement. In certain embodiments of the invention the cell is not an NK T cell. In certain embodiments of the invention the cell is a conventional T cell. In some embodiments of the invention a non-human mammal, e.g., a mouse, that has an at least partly humanized immune system, is used. For example, the mouse may be a transgenic mouse that contains one or more human transgenes, e.g., a human CD4, CD8, MHC, TAP, TCR, or BCR transgene. For example, the T or B cell may be obtained from such a mouse or a recipient may be transgenic for CD4, CD8, MHC, TAP, TCR, or BCR. In some embodiments of the invention a recipient blastocyst or embryo obtained from non-human animal that is deficient for RAG1 and/or RAG2, is used, e.g., as a recipient for an ES or iPS cell. The recipient may have a disruption/mutation of the gene encoding RAG1 and/or RAG2. Because such disruption/mutations block lymphocyte maturation, all mature lymphocytes in a chimera should be derived from the donor cells. In some embodiments a chimeric animal obtained according to the methods of the invention and having germline contribution from the original T or B cell is crossed with a RAG-deficient or otherwise immunodeficient animal, such that the lymphocytes of the offspring are derived from the chimeric animal and/or so that rearrangement of any unrearranged TCR or BCR locus is inhibited. It will be appreciated that disruption/mutation of other genes besides RAG1/RAG2 could be used. In some embodiments of the invention the donor animal from which T or B cells are isolated is an inbred animal, e.g., mouse. In some embodiments of the invention the donor animal from which T or B cells are isolated is an outbred animal, e.g., mouse. In some embodiments of the invention the recipient oocyte, embryo, blastocyst, or animal is an inbred animal, e.g., mouse. In some embodiments of the invention the recipient oocyte, embryo, blastocyst, or animal is an outbred animal, e.g., mouse. Mouse strains of interest include, e.g., Balb/c, Black6, C57BL/6, DBA/1, DBA/2, ICR, NOD, 129 strains such as 129/Sv, etc., and mice derived by crossing such strains, e.g., F1 or F2 mice.

In some embodiments of the invention animals that have a mutation or polymorphism in a gene of interest are used, e.g., as T or B cell donors or as recipients, or to backcross a chimeric or cloned animal of the invention. In some embodiments of the invention animals that have, overexpress, or underexpress a gene of interest are used, e.g., as T or B cell donors or as recipients or to backcross a chimeric or cloned animal of the invention. The gene of interest may be, e.g., a gene that is known or suspected to play a role in immune system development or function and/or in resistance or susceptibility to disease or infection. For example, the gene could encode a cytokine, chemokine, cytokine or chemokine receptor, transcription factor, growth factor, growth factor receptor, Toll-like receptor, kinase, phosphatase, etc. The gene, mutation, or polymorphism, or the overexpression or underexpression may be naturally occurring or engineered. For example, the animal may be a knockout animal, or may be transgenic for a short hairpin RNA (shRNA) that silences gene expression. The mutation may be generated using homologous or nonhomologous recombination. The mutation may disable the gene or may activate it or alter its function. Expression of the engineered gene could be conditional or constitutive. In some embodiments a transgene is targeted to a genetic locus that is not required for normal development of a mouse.

In some embodiments the engineered gene comprises expression control element(s), e.g., a promoter or promoter/enhancer, operably linked to a nucleic acid that encodes an RNA or polypeptide. In some embodiments the engineered gene comprises tissue-specific or cell-type specific expression control elements, so that the gene is expressed selectively in one or more cell types or tissues relative to others (e.g., in lymphoid cells, e.g., T or B cells). In some embodiments the gene comprises regulatable expression control element(s), e.g., an inducible or repressible promoter. Examples of regulatable promoters include heat shock promoters, metallothionein promoter, and promoters that comprise an element responsive to a small molecule such as tetracycline or a related compound (e.g., doxycycline), or a hormone. It will be understood that the cell should express the appropriate trans-acting proteins, e.g., proteins typically comprising a DNA binding domain, activation or repression domain, and ligand-binding domain to render transcription responsive to the ligand.

In some embodiments the animal is transgenic for a gene that encodes a marker protein, reporter, or genetically encoded sensor, e.g., one that would allow detection of one or more cell types or detection of a process or event or metabolite, etc. Exemplary marker proteins include fluorescent proteins such as green fluorescent protein (GFP), blue, sapphire, yellow, red, orange, and cyan fluorescent proteins and fluorescent variants such as enhanced GFP (eGFP), mCherry, etc., and luminescent proteins such as luciferase (e.g., firefly or Renilla luciferase), aequorin. In some embodiments the animal comprises a transgene that encodes a fusion protein that comprises a polypeptide of interest and a marker protein. In some embodiments the animal comprises a transgene that encodes a fusion protein that comprises a polypeptide of interest and a tag, e.g., an epitope tag that can be conveniently used for detection or purification. A transcriptional reporter could comprise a nucleic acid encoding a marker protein wherein the nucleic acid is operably linked to promoter of interest. A variety of genetically encoded sensors are known (Deuschle, K, et al. Cytometry A. 64(1):3-9, 2005). Such markers, reporters, or sensors could be used for in vitro or in vivo imaging, or to study events associated with B or T cell activation, e.g., phosphorylation of particular proteins, calcium release, nuclear translocation of particular proteins, or transcription of particular genes.

A variety of methods may be used to isolate a T or B cell having a predefined specificity. In some embodiments, a non-human animal, e.g., a mouse, is contacted with an epitope or antigen of interest. The contacting can be carried out in a variety of different ways and by a variety of different routes. For example, the epitope or antigen can be administered by injection (e.g., intraperitoneal, subcutaneous, intravenous, intradermal, etc.), by inhalation, orally, mucosally (i.e., by contacting a mucosal surface with the epitope or antigen). The epitope or antigen can be provided as part of a composition, which may comprise a variety of substances such as one or more adjuvants (see discussion below). The composition could comprise a purified epitope or antigen, or could comprise a cell or tissue extract comprising the epitope or antigen. In some embodiments the animal is contacted with an organism, e.g., a microorganism or multicellular parasite, comprising an epitope or antigen of interest. The microorganism or multicellular parasite could be viable or non-viable. In some embodiments the organism is capable of replication in the animal. In some embodiments the microorganism or multicellular parasite is “wild-type” while in other embodiments it is mutant, genetically modified, attenuated, or otherwise not wild type. In some embodiments the microorganism or parasite is drug-resistant. In some embodiments the composition comprises DNA or RNA that encodes an epitope or antigen of interest. The DNA or RNA could be delivered in a vector, e.g., a viral vector. The DNA or RNA is expressed in the animal, resulting in synthesis of the encoded epitope or antigen in the animal. The epitope, antigen, composition, or organism can be administered multiple times. In some embodiments, cells are harvested from the animal after a predetermined time period. For example, in some embodiments cells are harvested between 5 days and 60 days after initially contacting the animal with the epitope, antigen, composition, or organism. In some embodiments, cells are harvested about 2, 3, 4, 5, or 6 weeks after contacting the animal with the epitope, antigen, composition, or organism. In some embodiments, cells are harvested after the animal produces a detectable antibody titer against the epitope or antigen of interest. In some embodiments a typical or standard immunization schedule is used. In some embodiments cells are harvested less than 5 days, or more than 60 days, after initially contacting the animal with the epitope, antigen, composition, or organism

Cells can be isolated, e.g., from blood, lymphoid organs (e.g., spleen, lymph node), liver, or any organ or tissue or site of interest in the body. If desired, T or B cells can be separated from a total cell population by staining for or otherwise identifying cells that express characteristic cell surface molecules, e.g., CD4 or CD8 (for T cells), or CD19 or CD20 (for B cells), etc. For example, cells can be contacted with a labeled antibody that binds to the cell surface molecule, and the labeled cells separated from the unlabeled cells.

Cells are contacted (e.g., in a liquid medium) with an epitope or antigen of interest in order to identify those that have a specificity for such epitope or antigen. The epitope or antigen may be soluble or immobilized. The epitope may be presented in a complex or as part of a larger molecular entity (e.g., as part of a larger protein). The epitope or antigen can be presented as a multimer or complex comprising multiple antigen molecules, which can be crosslinked to each other or to another moiety. Cells that bind to the epitope, epitope-containing complex, or antigen, are isolated.

MHC/epitope complexes can be produced in vitro and used to directly identify and isolate T cells that specifically recognize a particular epitope of interest (see, e.g., Examples 1 and 2 and references therein). Such artificial MCH/epitope complexes typically include multiple MHC molecules, e.g., dimers, tetramers, at least some of which contain the epitope of interest. Recently, a method in which class I MHC molecules are occupied transiently with a conditional ligand that self-cleaves into two fragments upon photolysis has been described. When caged MHC-tetramers are exposed to a large molar excess of a ligand of choice during photocleavage, tetramers of desired specificity are generated, provided the putative ligands can bind to the class I MHC in question. This strategy allows the use of a single batch of photocleavable MHC tetramer from which arrays of MHC tetramers of defined specificity can be rapidly generated, e.g., for the purpose of high-throughput screening for T cell epitopes. As described in Example 1, a conditional ligand for both murine H-2Kb and H-2Db molecules of C57BL/6 mice for use in generating MHC multimers was developed. Without being bound by theory, the availability of a single conditional ligand for both these class I MHC products allows the phenotypic analysis of all CD8+ T lymphocytes that undergo clonal expansion after antigenic challenge in C57BL/6 mice. This approach was used to identify T cell epitopes from C. trachomatis and T. gondii and can readily be extended to other microorganisms or antigens of interest.

The epitope, epitope-containing complex, or antigen may comprise a label or have a label attached thereto, e.g., to facilitate isolation of cells that have bound the epitope, complex, or antigen. In various embodiments of the invention a label can be covalently or noncovalently attached to the epitope, complex, or antigen, either directly or indirect (e.g., both can be attached to a third moiety).

In some embodiments a T or B cell having (or not having) one or more properties of interest, in addition to specificity for a predetermined epitope or antigen, is selected. For example, it may be of interest to select a B cell that expresses a particular immunoglobulin isotype (e.g., IgG, IgM) or subclass (e.g., IgG1, IgG2, IgG3, IgG4), Such cells can be isolated, e.g., using a binding agent (e.g., an antibody) that binds to the constant region of an antibody of the particular isotype or subclass. It may be of interest to select a CD4⁺ or CD8⁺ T cell or a particular CD4+ T cell subset. Such selection can be performed based on particular cell surface markers using flow cytometry or other methods.

A label often comprises or consists of a compound that can be directly or indirectly detected, e.g., visually or using suitable instrumentation. The label is often an optically detectable label, e.g., a compound that produces a signal or a change in a signal based on light or an interaction with light. The signal can be, e.g., light scattering, absorption, emission, polarization, etc. Exemplary labels include fluorescent or luminescent molecules such as acridine dyes, Alexa dyes, cyanine dyes, fluorescein and derivatives thereof, rhodamine and derivatives thereof, particles such as quantum dots, etc. See, e.g., “Handbook of Fluorescent Probes and Research Products” (Molecular Probes, 9th edition, 2002) and “The Handbook—A Guide to Fluorescent Probes and Labeling Technologies”, (Invitrogen, 10th edition, available at the Invitrogen web site). Such labels are of use, e.g., to separate cells using FACS. In some embodiments a label comprises a metal, such as gold. In some embodiments a label comprises a magnetic moiety, e.g., a superparamagnetic moiety. In some embodiments a label comprises, or is attached to, a particle. For example, magnetic particles, often referred to as “beads”, are of use for cell separation. The particles typically have a moiety attached thereto that binds to a marker on the cell surface. Cells labeled with magnetic particles can be separated from non-labeled or weakly labeled cells using a magnetic field. Such particles may be biodegradable and/or may be readily removable from the cells, e.g., by cleavage. In some embodiments, a label comprises a moiety that can be used for affinity-based cell separation. For example, a label can comprise a moiety that is recognized by an antibody or other binding agent.

Cells can be sequentially or simultaneously contacted with multiple agents for purposes of selecting a desired cell. For example, cells can be contacted with a labeled antibody that binds to a characteristic cell surface molecule and with an MHC/epitope complex. Positive or negative selection, or both, can be used. Cell separation protocols can be based in part on physical properties of the cells, such as size or density. For example, cells can be passed through or trapped by strainers or filters. Density gradient centrifugation can be used. A variety of reagents and kits useful for cell separation are commercially available, e.g., kits using MACS® technology, comprising MACS MicroBeads, manual or automated MACS Separators, and/or MACS Columns (Miltenyi Biotec GmbH, Bergisch Gladbach, Germany), Dynabeads® produts and technology (Life Technologies Corporation, Carlsbad, Calif., formerly Invitrogen Corp.), etc. See Kumar, A., et al. (eds.), Cell Separation: Fundamentals, Analytical and Preparative Methods (Advances in Biochemical Engineering/Biotechnology), Springer, 2007, ISBN-13: 978-3540752622) for a description of various cell separation techniques. The cell separation methods can be used to identify T or B cells for use to generate transnuclear mice and/or to isolate and analyze cells from such mice or from control mice.

A variety of methods may be used in the present invention to generate a non-human animal, e.g., mammal, from a T or B cell. In some embodiments, the method comprises performing direct cloning by performing SCNT, activating the resulting oocyte, allowing the oocyte to begin development (e.g., to the blastocyst stage), and then transferring the resulting organism (e.g., blastocyst) into a pseudopregnant female. In some embodiments, the method comprises performing indirect cloning by performing SCNT, generating a blastocyst, isolating ES cells from the blastocyst, and using the ES cells to generate an animal (e.g., by introducing the ES cell into a blastocyst and implanting the blastocyst into a pseudopregnant female). See, e.g., Wakayama, T., et al., Nature, 394(6691): 369-74 (1998); Wakayama, T. and R. Yanagimachi, Mol Reprod Dev., 58(4):376-83 (2001) for examples of SCNT technology applied to cumulus cells and fibroblasts. It will be appreciated that variations exist. For example, embryos can be cultured and transferred into recipient females at the two cell or morula stage in certain embodiments. In some embodiments the resulting animal is a chimeric animal. In some embodiments tetraploid complementation is used, wherein the ES cell is transferred into a tetraploid blastocyst, wherein the placenta is derived from the tetraploid host cells and the embryo from the injected donor ES cells. The resulting animal contains cells derived primarily from the ES cell (and thus from the original T or B cell). See, e.g., U.S. Pat. No. 6,784,336 and Hochedlinger, K. and R. Jaenisch, Nature, 415(6875):1035-8 (2002). A similar approach could be used for iPS cells. In some embodiments ES cells are introduced into eight cell-stage embryos, e.g., using laser-assisted injection (Poueymirou, W., et al., Nature Biotechnology, 25(1): 91-99, (2007). In some embodiments, cell fusion, e.g., with an existing ES cell is used to reprogram a T or B cell with a predefined specificity.

In some embodiments the method of generating a non-human animal comprises using a compound that inhibits histone deacetylation, e.g., a histone deacetylase inhibitor, during at least part of the procedure. Exemplary histone deacetylase inhibitors are small chain fatty acids (e.g., valproic acid or butyrate); hydroxamate small molecule inhibitors (e.g., SAHA and PXD101); other small molecule inhibitors, e.g., MS-275; various cyclic peptides such as depsipeptide; trichostatin A (TSA); apicidin, etc. For example, in some embodiments the method using SCNT comprises using TSA (e.g., at about 5 nM to 100 nM) in oocyte activation medium and/or medium in which activated oocytes are cultured. TSA may be used, e.g., for between 6-10 hours in exemplary embodiments, such as 6 hours during activation followed by an additional 4 hours.

It has recently been shown that mouse and human fibroblasts can be reprogrammed in vitro to a pluripotent state through retroviral-mediated introduction of combinations of transcription factors, e.g., the four transcription factors Oct4, Sox2, Klf4, and c-Myc (with c-Myc being dispensable, although omitting c-Myc reduced reprogramming efficiency), or the four transcription factors Oct4, Nanog, Sox2, and Lin28 (see, e.g., Meissner, A., et al., Nat Biotechnol., 25(10):1177-81 (2007); Yu, J., et al, Science, 318(5858):1917-20 (2007); and Nakagawa, M., et al., Nat Biotechnol., 26(1):101-6 (2008), referred to as “reprogramming factors”). The resulting cells, termed iPS cells, appear essentially identical to ES cells, and can be used to generate viable chimeras with contribution to the germ line. It was further shown that non-fully and fully differentiated mouse B lymphocytes can be reprogrammed to pluripotency using similar approaches involving additional interruption with the transcriptional state maintaining B cell identity by either ectopic expression of the myeloid transcription factor CCAAT/enhancer-binding-protein-alpha (C/EBPalpha) or specific knockdown of the B cell transcription factor PaxS (Hanna, et al., Cell, 133(2):250-64 (2008). Furthermore, it has been reported that certain small molecules can enhance the reprogramming process. See, e.g., Shi, Y., et al., Cell Stem Cell, 2: 525-528 (2008); Huangfu, D., et al., Nature Biotechnology; Published online: 22 June 2008|doi:10.1038/nbt1418. The invention encompasses use of such molecules or others, e.g., histone deacetylase inhibitors, methyltransferase inhibitors, Wnt pathway agonists, molecules that enhance expression of endogenous genes such as Oct4, Sox2, etc., in the methods of the invention, or molecules that can substitute for one or more reprogramming factors. See, e.g., PCT/US2008/010249 (WO/2009/032194) and PCT/US2008/004516 (WO/2008/124133); Lysiottis, et al., Proc Natl Acad Sci USA. 106(22):8912-7, 2009.

The present invention encompasses in vitro reprogramming of a T or B cell with a predefined specificity using any available in vitro reprogramming technique to generate an iPS cell from the T or B cell. The invention further encompasses use of such iPS cells to generate cloned or chimeric non-human mammals containing T or B cells of the predefined specificity. The invention also encompasses isolating T or B cells from a non-human mammal suffering from a condition of interest, e.g., cancer or an autoimmune disease or an infection, generating iPS cells from such T or B cells, and using such iPS cells to generate a cloned or chimeric animal.

An important aspect of certain embodiments of the invention is to sort or otherwise identify a T or B cell of predefined specificity for use in the methods of generating a non-human animal. An important aspect of certain embodiments of the invention is to use T or B cells obtained from an animal suffering from a condition of interest, e.g., cancer or an autoimmune disease or infection (e.g., by a microorganism or multicellular parasite), wherein, for example, it is of interest to analyze T or B cells in subjects suffering from the condition or wherein it is of interest to generate a non-human mammal from such T or B cells. The invention is distinct from approaches in which T or B cells having undefined and unknown specificity (e.g., T or B cells obtained from blood, spleen, liver, etc., e.g., obtained from a normal non-human mammal, and not further evaluated or sorted for their binding specificity) are used to generate non-human mammals, ES cells, iPS cells, etc.

In some embodiments of the invention T or B cells are subjected to the initial steps of an SCNT protocol (e.g., harvesting of the nucleus) or reprogramming protocol within a predetermined time period following their isolation from an animal and/or following their identification as being specific for an epitope or antigen of interest. For example, the T or B cells may be sorted and used within, e.g., 2 hours of being sorted. In some embodiments T or B cells are used within up to 3, 4, 6, 8, or 12 hours of being isolated from an animal. In some embodiments the T or B cells are sorted and are not returned to standard culture conditions (e.g., incubation at ˜37 degrees C. in cell culture medium) prior to use. In other embodiments, cells may be returned to culture and subsequently used. While Applicants were not able to derive SCNT-ES cell lines from T cells that had been cultured for between 1 and 7 days in their initial experiments, it is anticipated that modification of the culture conditions and/or maintaining the cells in culture for longer time periods would allow such derivation.

The present invention provides a convenient means to generate mice or other non-human mammals carrying a specific TCR or BCR without use of transgenic techniques and, in some embodiments, without genetic modification. In certain embodiments of the invention the TCR and BCR genes retain their endogenous regulatory elements and are located at their native position in the genome rather than being located at non-homologous sites. Without wishing to be bound by any theory, for this and other reasons, various embodiments of the instant invention offer a number of advantages relative to mice known in the art that are transgenic for rearranged T cell receptor or B cell receptor genes. The generation of such transgenic models is based on the long-term culture and repeated stimulations with antigen either as a T cell clone or as a T cell hybridoma, therefore most likely selecting cells that survive the culture conditions best. Once a line or clone has been established, the TCR α- and β-chains are isolated and cloned either as cDNA or genomic fragment under the control of a non-endogenous promoter and integrated into the mouse genome at non-homologous sites. Although several expression cassettes for the TCR transgenes are available, the generation of such mice remains a challenge. Random integration and non-endogenous promoter inevitably leads to variation in expression levels and kinetics, even among mice expressing the same TCR (27). Further studies of a suitable line are dictated mostly by random selection of a “best responder”. The variations in expression level and kinetic of a transgenic TCR can strongly influence the development of T cells. As described in the Exemplification, Applicants showed that certain transnuclear mice not only express their TCR in a more homogenous fashion among each other but their development mimics a more physiological pattern than transgenic mice.

In some embodiments of the invention a non-human animal generated from a T or B cell having a predefined specificity is a chimeric animal. In some embodiments of the invention a non-human animal generated from a T or B cell having a predefined specificity is a cloned animal. In some embodiments of the invention the animal is backcrossed, e.g., to a wild type animal. It will be appreciated that the non-human animal of the invention need not be derived solely from the original T or B cell but in certain embodiments of the invention may also have a contribution (e.g., a genetic contribution) from the recipient and/or from an animal with which a chimeric animal is back-crossed. For example, and without limitation, an enucleated oocyte contributes cytoplasm, mitochondria, etc., and a recipient blastocyst contributes cells, etc.

In some embodiments of the invention at least 20%, at least 30%, at least 40%, at least 50%, at least 60%, at least 70%, at least 80%, or more of the T or B cells of the non-human animal of the invention (which can be chimeric or entirely derived from such T or B cells or backcrossed to a strain of interest) have the predefined specificity of the original T or B cell. For example, in some embodiments between 30% and 60%, or in some embodiments between 60% and 80% of the T or B cells in an animal having both rearranged TCR or BCR chains derived from the original T or B cell have the specificity of the original T or B cell. In some embodiments between 80% and 90% or between 90% and 95%, 96%, 97%, 98%, 99%, or more of the T or B cells in an animal having both rearranged TCR or BCR chains derived from the original T or B cell have the specificity of the original T or B cell.

The non-human animals, e.g., mammals, of the invention, and cells obtained therefrom, have a variety of uses. For example, such animals can be used as animal models to study development and function of the immune system or components thereof, e.g., T cells or B cells. Once a non-human mammal of the present invention is produced, cells can be isolated from the animal. Cells could be isolated from any organ or tissue. For example, cells could be isolated from blood, lymphoid tissue (e.g., spleen, lymph node), bone marrow, liver, etc. Optionally, the cells are further analyzed or characterized in vitro. For example, the cells can be contacted with a MHC tetramer complex containing the epitope of interest and sorted by FACS to isolate T cells specific for the epitope from among a population of cells. Alternately B cells could be contacted with the epitope or antigen itself In some embodiments, plasma cells that secrete an antibody specific for the epitope or antigen of interest are isolated. The tetramer complex, epitope, or antigen could be labeled (e.g., fluorescently labeled) or tagged to facilitate isolation of cells that bind to it. The non-human animals of the invention thus have, in various embodiments, the capacity to serve as a source of T and/or B cells having a predefined specificity for an antigen or epitope of interest, or as a source of T and/or B cells that may promote or at least in part cause an undesired immune response, or as a source of T and/or B cells that may promote or at least in part provide a beneficial immune response (e.g., a protective, therapeutic, or curative immune response). The invention provides a population of T or B cells, wherein the T or B cells are isolated from an animal produced according to the invention. In some embodiments, the invention provides a population of T or B cells having a predefined specificity, wherein the T or B cells are isolated from an animal produced according to the invention. In some embodiments, a cell line, e.g., an immortalized cell line, is generated from such T or B cells.

In some embodiments, animals of the invention, e.g., animals generated from a T or B cell with predefined specificity, are immunized with the epitope or antigen or are infected with a microorganism or multicellular parasite. Cells are harvested at various time points and their response to the epitope, antigen, or microorganism is assessed. Alternately, animals are not immunized. T or B cells are harvested and may be contacted with an epitope or antigen of interest in vitro and their response assessed.

If desired, the rearranged TCR or BCR of the T or B cells (or antibody secreted by plasma cells) can be cloned, sequenced, and/or produced in vitro using recombinant DNA technology. It may be of interest to analyze the details of the interaction of the epitope or antigen with the TCR, BCR, or antibody, e.g., by modeling or crystallizing the complex. In some embodiments, T or B cells are isolated and used for adoptive transfer to a host animal, e.g., of the same species. Animals of the present invention can also serve as a source of antibody of a predefined specificity. The invention thus provides a method of producing an antibody comprising generating a non-human mammal from a mature B cell having a predefined specificity, wherein the animal produces the antibody with a predefined specificity, and harvesting the antibody from the animal.

Non-human mammals of the invention, such as mice, can be used as a model for a condition for which a preventive or therapeutic agent is sought. In some embodiments of the invention the condition is an autoimmune disease. For example, non-human mammals of the present invention in which the T or B cells have specificity for a self antigen may serve as a model of autoimmune disease. Non-human mammals of the present invention in which T or B cells have specificity for an allergen may serve as a model for an allergic or atopic condition. In some embodiments, animals of the present invention that were derived from T or B cells obtained from an animal suffering from or at risk of the condition serve as a model of the condition.

A method of identifying an agent to be administered to treat a condition in a mammal comprises producing, using the methods of the present invention, a non-human mammal, e.g., a mouse that is a model of the condition; administering to the animal mouse an agent, referred to as a candidate agent, to be assessed for its effectiveness in treating or preventing the condition; and assessing the ability of the agent to treat or prevent the condition. If the candidate agent reduces the extent to which the condition is present or progresses or causes the condition to reverse (partially or totally), the candidate agent is an agent to be administered to treat the condition. Agents that may be tested could be, for example, small molecules, proteins, nucleic acids, etc. “Small molecule” refers to organic compounds, typically containing multiple carbon-carbon bonds, having a molecular weight of 2,500 daltons or less, e.g., 2,000 daltons or less, e.g., 1,500 daltons or less, e.g., 1,000 daltons or less. In some embodiments the small molecule has between 5 and 50 carbon atoms, e.g., between 7 and 30 carbons, and often one or more heteroatoms, e.g., nitrogen, oxygen, sulfur. In some embodiments, the agent is a candidate vaccine or vaccine component. The vaccine or vaccine component may comprise the epitope or antigen for which the T or B cells from which the non-human mammal was derived has specificity. The technique may be used determine whether the presence of T or B cells with a certain specificity is of advantage or disadvantage for the outcome of the underlying disease and/or to assess the effect of a particular vaccine or vaccine component on such cells or on the animal's immune response, e.g., its ability to withstand infection. The invention thus provides a means to evaluate the efficacy or effect of a candidate vaccine or vaccine component. The invention provides methods to assess or investigate any vaccine or vaccine component of interest. In some embodiments a vaccine or vaccine component of interest comprises an adjuvant (i.e., a substance that stimulates or promotes the immune response towards a co-administered antigen), or a substance to be assessed for use as an adjuvant. Exemplary adjuvants include inorganic compounds such as aluminum salts, organic compounds such as squalene, oil-based adjuvants such as complete or incomplete Freund's adjuvant, ISCOMATRIX (a particulate adjuvant comprising cholesterol, phospholipid and saponin), virosomes, TLR ligands such as oligonucleotides comprising CgG motifs, double-stranded RNA, bacterial cell wall components such as lipopolysaccharide, bacterial exotoxins such as E. coli heat-labile enterotoxin (“LT”), (U) cholera toxin (“CT”), or diphtheria toxin (“DT”) or detoxified mutants of any of these. In some embodiments the invention comprises a method of assessing a composition comprising an antigen and an adjuvant. For example, it may be of interest to assess the response to different compositions comprising a particular antigen and different adjuvants, e.g., to identify adjuvants particularly suited for use with a particular antigen. In some embodiments a vaccine comprises a vector, e.g., a viral vector, that delivers a DNA or RNA that encodes an epitope or antigen of interest to the animal. Exemplary viral vectors include, e.g., retroviruses (e.g., lentiviral vectors), adenoviral vectors, and adeno-associated viral vectors. A viral vector could comprise an intact virion or a portion thereof, such as at least a portion of the viral genome.

It is also of interest to assess the effect of particular agents on immune system function in animals of the invention. For example, animals of the present invention represent an attractive system to study the effect of known or potential immunodulators, e.g., immunosuppressive agents, on immune system development function. Furthermore, animals of the present invention are of use to study the effect of mutations, polymorphisms, overexpression, or underexpression of various genes on immune system development and function.

In some embodiments, an animal of the invention is used to assess whether a particular epitope or antigen contributes to development or progression of a disease. For example, an animal is generated from a T or B cell having predefined specificity for an epitope or antigen to be assessed, e.g., an epitope or antigen suspected of playing a role in autoimmune disease (e.g., suspected of being a target of the immune system in individuals suffering from the disease). The animal is observed to determine whether it develops the disease and/or the progression of the disease is monitored. In some embodiments the animal is immunized with the antigen. The animal may be used to assess candidate agents for use in prevention or therapy of the disease. In some embodiments the animal is repeatedly exposed to the epitope or antigen.

In some embodiments of the invention, a non-human animal is generated from a T or B cell obtained from an animal that serves as a model for a condition. For example, the animal may be one that suffers from an autoimmune disease or cancer or an allergic condition. Exemplary autoimmune diseases are diabetes mellitus (type I), multiple sclerosis, rheumatoid arthritis, systemic or cutaneous lupus erythematosus, Wegener's granulomatosis, Goodpasture's disease, ankylosing spondylitis, autoimmune hepatitis, thyroiditis, Graves' disease, myasthenia gravis, etc. Animal models of a number of these diseases are known. See, e.g., Taneja, V. and David, C. S., Nature Immunology, 2(9): 781-784 (2001). For example, the non-obese diabetic mouse, or the experimental autoimmune encephalomyelitis (EAE) model of multiple sclerosis, are of interest. Asthma is another condition of interest. The invention provides a non-human mammal obtained by crossing a non-human mammal derived from a T or B cell that has predefined specificity with a non-human mammal of the same species, wherein the non-human mammal of the same species serves as a model of human disease.

In some embodiments, an animal of the invention is used to assess whether a particular epitope or antigen contributes to protection against development or progression of a condition. For example, an animal is generated from a T or B cell having predefined specificity for an epitope or antigen suspected of playing a role in protecting against development or progression of the condition (e.g., suspected of being a target of the immune system in individuals suffering from or at risk of the condition. In some embodiments the epitope or antigen is derived from a tumor or from a pathogenic microorganism or multicellular parasite. The animal is observed to determine whether it develops the condition and/or progression or severity of the condition is monitored. In some embodiments the animal is immunized with the antigen or infected with the pathogenic organism. In some embodiments, tumor tissue or tumor cells (e.g., primary tumor cells, tumor cell lines, etc.) are introduced (e.g., injected, implanted) into the animal. The animal may be used to assess candidate agents for use in prevention or therapy of the condition. In some embodiments an animal derived from a T or B cell having specificity against a tumor antigen is implanted or injected with tumor tissue, tumor cells, tumor cell line (e.g., comprising cells expressing the antigen). The animal is monitored to assess tumor growth and/or metastasis. If the tumor growth or metastasis occurs to a lesser extent in the TN animal than in a control animal, the tumor antigen or an epitope thereof is a candidate for development of an anti-tumor vaccine.

Animals, e.g., mice, derived using the inventive methods may be used, e.g., to address questions such as whether responses against various different epitopes are equally protective, or whether there a distinction among such epitopes; whether certain epitopes are more likely to elicit particular subsets of cells, e.g., memory T cells and others effector T cells; whether responses against some epitopes protect from particular manifestations of a disease, e.g., Toxoplasma-induced encephalitis, whereas others do not; whether responses against some epitopes can be used not only as prophylaxis, but also to clear an established infection, and many others.

In the methods described herein, the non-human animal may be compared with a suitable control animal of the same species. The control animal may be isogenic with the non-human animal of the invention, except for its TCR or BCR. The control animal may be, e.g., an animal generated from a T or B cell that does not have specificity for the predefined epitope or antigen. The control animal may be generated via normal reproduction without use of SCNT or in vitro somatic cell reprogramming and is typically not descended from an animal generated using such techniques.

The methods described herein may be provided as a service in which non-human mammals, e.g., mice, ES cells, and/or iPS cells are generated (e.g., for a fee) upon request of researchers or companies. The invention provides a method of doing business comprising receiving a request (e.g., an “order”) for or relating to a non-human mammal, ES cells and/or iPS cell; generating a non-human mammal, ES cell, and/or iPS cell according to the method of the invention; and providing the non-human mammal, ES cell, and/or iPS cell in response to the request and/or performing further studies using the non-human mammal, ES cell, and/or iPS cell. In some embodiments, the non-human mammal, ES cell, and/or iPS cell is derived from a T or B cell having a predefined specificity. The invention provides a method of doing business comprising receiving a request (e.g., an “order”) for or relating to a T or B cell having a predefined specificity; generating a non-human mammal according to the method of the invention using a T or B cell of the predefined specificity; and providing T or B cells harvested from the non-human mammal in response to the request and/or performing further studies using the T or B cell(s) having a predefined specificity.

The practice of the present invention will employ, unless otherwise indicated, conventional techniques of mouse genetics and manipulation, cell biology, cell culture, molecular biology, transgenic biology, microbiology, recombinant DNA, and immunology, which are within the skill of the art. Such techniques are described in the literature. Non-limiting descriptions of certain techniques are found in the following publications: Ausubel, F., et al., (eds.), Current Protocols in Molecular Biology, Current Protocols in Immunology, Current Protocols in Protein Science, and Current Protocols in Cell Biology, all John Wiley & Sons, N.Y., e.g., edition as of July 2008 or before; Sambrook, Russell, and Sambrook, Molecular Cloning: A Laboratory Manual, 3rd ed., Cold Spring Harbor Laboratory Press, Cold Spring Harbor, 2001; Harlow, E. and Lane, D., Immunochemical Protocols (Methods in Molecular Biology) Humana Press; 3rd ed., 2005. All patents, patent applications and references cited herein (including those cited in Examples 1 and 2 and elsewhere in the Exemplification) are incorporated herein in their entirety by reference. In the event of a conflict or inconsistency between any of the literature and the instant specification, the specification (and any amendments thereto) shall control. Standard art-accepted meanings of terms are used herein unless indicated otherwise. Standard abbreviations for various terms are used herein.

### EXEMPLIFICATION

The invention now being generally described, it will be more readily understood by reference to the following example, which are included merely for purposes of illustration of certain aspects and embodiments of the present invention, and are not intended to limit the invention in any way. It should be understood that the epitopes and reagents identified as described in the Examples, compositions containing them, and methods of making and using them, are aspects of the invention. For example, and without limitation, C. trachomatis and T. gondii epitopes identified herein may be of interest to study immune system response to infection, as potential protective epitopes (e.g., when used as vaccines), and such compositions and uses are a distinct aspect of the invention and independent from the non-human animals and cells of the invention.

### Example 1

### Discovery of CD8+ T Cell Epitopes in Chlamydia trachomatis Infection and Isolation of CD8+ T Cells with Specificity These Epitopes

This example describes the development of photocleavable analogs of the FAPGNYPAL (SV9) epitope that bind H-2K(b) and H-2D(b) with full retention of their structural and functional integrity. A 2,000-member class I MHC tetramer array containing nonameric epitopes that span the genome of Chlamydia trachomatis was produced. The array allowed the discovery of two variants of an epitope derived from polymorphic membrane protein I (PmpI) and the isolation of CD8+ T cells specific for these epitopes. Details of this example are contained in the paper entitled, “Discovery of CD8+ T cell epitopes in Chlamydia trachomatis infection through use of caged class 1 MHC tetramers”, Grotenbreg G M, et al., Proc Natl Acad Sci USA, 105(10):3831-6, 2008. See U.S. Ser. No. U61/077,807 and U.S. Ser. No. 61/077,835.

### Example 2

### Identification of Toxoplasma gondii Epitopes and Isolation of CD8+ T Cells with Specificity for These Epitopes

Caged MHC molecules were used to generate ˜250 H-2Ld tetramers and distinguish T. gondii-specific CD8+ T cells in BALB/c mice. Two T. gondii specific H-2Ld-restricted T cell epitopes were identified, one from dense granule protein GRA4 and the other from rhoptry protein ROP7. H-2Ld/GRA4 reactive T cells were isolated from multiple organ sources and predominate 2 weeks after infection, while the reactivity of the H-2Ld/ROP7 T cells peaks 6-8 weeks after infection.

Toxoplasma gondii is an obligate intracellular parasite infecting all homeothermic vertebrate hosts, with human infection rates of 20%-90%. As the causative agent of toxoplasmic encephalitis, the parasite poses a severe health threat for immunocompromised individuals, especially AIDS patients, and it causes congenital defects in newborns [1]. No human vaccines or drugs that eradicate the infection are available.

In humans and rodents, T. gondii exists as the rapidly multiplying lytic tachyzoites, which later during infection convert into slower growing bradyzoites, harbored in cysts in neural and muscular tissue [2]. Sequential secretion through 3 organelles—micronemes, rhoptries, and dense granules from tachyzoites—mediates invasion and survival inside the host cell [3]. In Europe and North America the prevalent T. gondii strains are divided into 3 types, with type II strains accounting for >70% of isolates obtained from human cases of toxoplasmosis [4].

Like immunocompetent humans [5], BALB/c mice limit type II and III T. gondii, with fewer cysts in their brains developing a latent chronic infection, compared with susceptible mouse strains [6]. This is ascribed to the generation of H-2L^{d}-restricted cytotoxic T lymphocytes and dependent on type II parasites [7]. When the immune response wanes, the parasite may recrudesce from the bradyzoite to the tachyzoite stage, which can invade virtually any nucleated cell. By replicating unchecked, T. gondii can cause fatal toxoplasmic encephalitis. The generation of interferon (IFN)-γ by innate NK cells and by CD4⁺ and CD8⁺ T lymphocytes is central to host resistance [8-10]; however, no T. gondii-derived CD8⁺ T cell epitopes have been reported prior to the instant invention. Published reports of T. gondii-specific CD8⁺ T cell responses are based on studies in which animals were vaccinated mostly with major parasite protein: the surface antigens (SAG1-SAG3). The resulting CD8⁺ T cells responded to either parasite lysate or to peptide restimulation in vitro ([11-14] and references therein). Whether these potential SAG epitopes are generated in the course of a natural infection is not known.

Problems inherent in the identification of parasite-derived T cell epitopes are the organisms' large and complex genomes, their multifaceted life cycles, and their persistence in the host despite the presence of protective immunity. We used the caged major histocompatibility complex (MHC)-tetramer technology [15-17] to generate an array of ˜250 H-2L^{d} tetramers of defined specificity to screen for T. gondii-specific T cell epitopes in infected BALB/c mice. We identified CD8⁺ T cell epitopes derived from 2 distinct parasite proteins, dense granule protein GRA4 and rhoptry protein ROP7. The GRA4-specific T cells are detected during the acute phase of the infection, whereas the T cells reactive to the ROP7 peptide persist during the chronic phase. Both types of T cells secrete protective IFN-γ. Mice infected with mutant parasites defective in establishing a chronic infection exhibit altered levels of the 2 epitope-reactive T cells throughout the course of infection, consistent with the ability of bradyzoites to sustain a ROP7-reactive CD8⁺ T cell response. The identification of endogenous T. gondii-derived epitopes, as distinct from the use of engineered T. gondii expressing model antigens, affords new opportunities for dissecting the immune response against the parasite.

Results

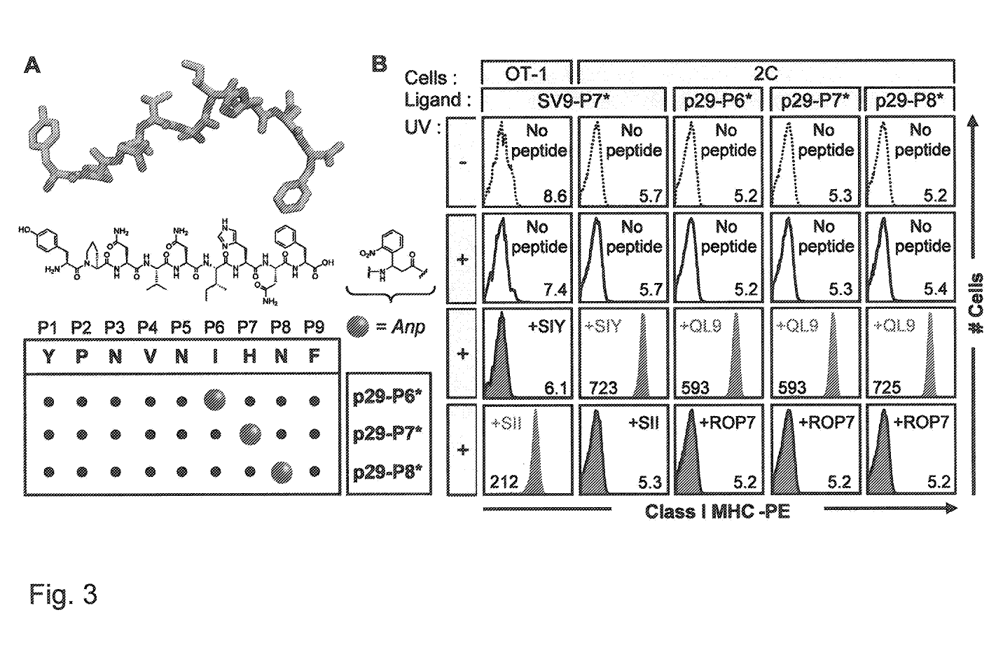

Design and validation of conditional ligands for H-2L^{d} class I MHC tetramers. MHC tetramers enable the direct visualization of antigen-specific T cells [26], and arrays of MHC tetramers can be produced rapidly by using caged MHC complexes. This technology is based on transient occupation of class I [15-17] or class II [27] MHC molecules with a conditional ligand. Here, we apply this strategy to the identification of parasite-specific CD8⁺ T cell epitopes by generating ˜250 distinct H-2L^{d} MHC tetramers. We designed 3 photocleavable ligands based on the p29 peptide, which conforms to the H-2L^{d} consensus binding motif [28]. After inspection of the crystal structure of H-2L^{d} in complex with p29 ([29] and FIG. 3A) we surmised that the Ile, His, and Asn amino acid residues at positions P6, P7, and P8, respectively, could be replaced with the photocleavable 3-amino-3-(2-nitro)phenyl-propanoic acid (Anp) residue. The resulting p29-P6*, p29-P7*, and p29-P8* ligands were synthesized and used for the production of caged H-2L^{d} tetramers.

To validate the peptide exchange reaction, we used 2C T cell receptor (TCR)-transgenic cells that recognize the H-2L^{d}/QL9 complex [30]. Only after UV irradiation in the presence of QL9 did we observe successful peptide exchange for the 3 H-2L^{d} tetramers preloaded with p29-based ligands as visualized by surface staining (FIG. 3B). Control staining that used an irrelevant H-2L^{d} ligand IPAAAGRFF (see below) established that staining of 2C T cells was strictly peptide-specific. We then applied these conditions to H-2K^{b} complexes carrying photocleavable SV9-P7* [16]. Here, UV-induced peptide exchange yielded reagents that stained both OT-1 and 2C TCR-transgenic cells when provided with the respective SIINFEKL and SIYRYYGL peptide, consistent with the ability of the 2C T cell clone to recognize both allogeneic (H-2L^{d}) and syngeneic (H-2K^{b}) peptide-MHC complexes [30]. The p29-P6*, p29-P7*, and p29-P8* photolabile peptides can thus be used as conditional ligands for H-2L^{d} to rapidly generate MHC tetramer arrays of defined specificity in a single step without loss of functional integrity. We arbitrarily chose to use H-2L^{d}/p29-P8* for screening purposes.

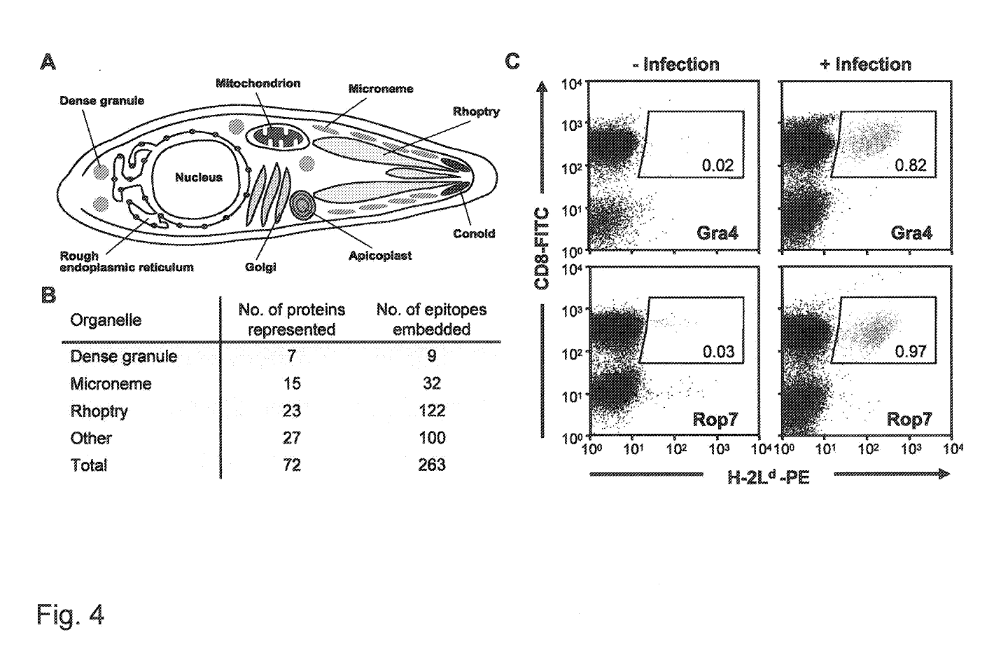

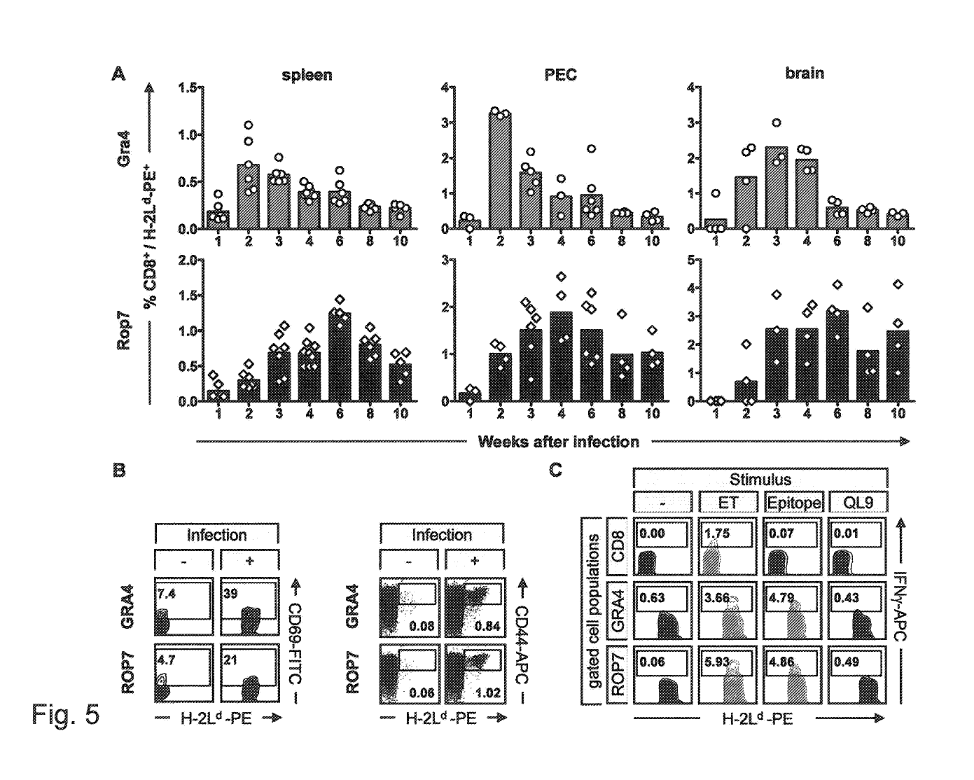

Screening for CD8⁺ T cell epitopes from T. gondii. A type II Pru line engineered to express the model H-2L^{d} antigen β-galactosidase, elicits a specific CD8⁺ T cell response that peaks 3 weeks after infection [31]. We therefore mined the T. gondii database for tachyzoite-stage secreted proteins to compile a partial list of antigens that could be a source of T. gondii CD8⁺ T cell epitopes (FIGS. 4A and 4B and U.S. Ser. No. U61/077,807 and U.S. Ser. No. 61/077,835). Three Web-based predictive algorithms were used to analyze 73 selected proteins for the presence of candidate H-2L^{d}-restricted nonameric epitopes. This gave 246 unique candidate sequences (FIG. 5B and U.S. Ser. No. U61/077,807 and U.S. Ser. No. 61/077,835) out of 33.525 possible nonameric peptides in this protein data set, which we then used to generate distinct H-2L^{d} tetramers.

Splenic CD8⁺ T cells from BALB/c mice were collected 10 and 21 days after intraperitoneal injection of 40 T. gondii ME49 cysts and stained with freshly generated H-2L^{d} tetramers. We found 2 different H-2L^{d}-restricted antigens in these independent screens; the peptide SPMNGGYYM, from GRA4, and IPAAAGRFF, derived from ROP7 (FIG. 4C).

Kinetics of H-2L^{d} tetramer—positive T cells throughout the infection. We found different frequencies and distribution for the GRA4- and ROP7-specific CD8⁺ T cell response. Infected BALB/c mice exhibited GRA4-reactive T cells as early as 10 days after infection (FIGS. 4C and 5A), whereas the ROP7 CD8⁺ T cell response peaked during week 6 after infection (FIG. 5A). For both epitopes the levels of tetramer-positive CD8⁺ T cells were 2-3 times higher in the peritoneal cavity (PEC) and brain than in the spleen. The parasite had converted to the bradyzoite stage, since we could detect cysts in fixed brain sections from a BALB/c mouse 4 weeks after infection. (data not shown). Moreover, both epitopes were not only generated in BALB/c mice infected intraperitoneally, but also in animals orally infected with 5 ME49 cysts as evident from the presence of the corresponding tetramer-positive CD8⁺ T cells (data not shown).

The T. gondii-specific CD8⁺ T cells displayed up-regulated early activation marker CD69 (FIG. 5B), compared with uninfected CD8⁺ T cell populations, as well as in comparison with tetramer-negative CD8⁺ T cells (data not shown). Surface expression of CD44 (FIG. 5B) was high, relative to that of the tetramer-negative CD8⁺ splenocytes, and persisted throughout the later stages of infection, consistent with their in vivo activation state. Upon restimulation in vitro, both the ROP7- and GRA4-specific CD8⁺ T cells produced IFN-γ in response to the corresponding peptide (FIG. 5C). Moreover, tetramer-reactive CD8⁺ T cells from chronically infected animals (week 8 after infection) displayed renewed expression of CD69 on their surface after 12 h of in vivo restimulation with 10⁵ T. gondii Pru tachyzoites (data not shown).

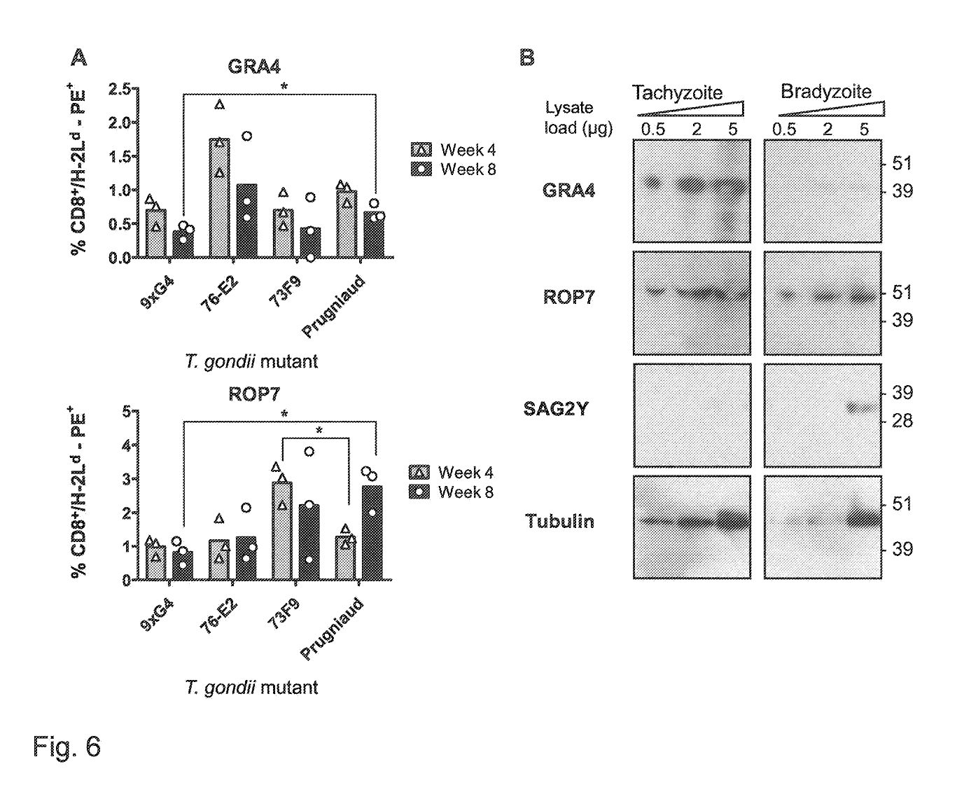

Parasites defective in establishing a chronic infection show altered levels of H-2L^{d}/ROP7-positive CD8⁺ T cells. To dissect the origin of the GRA4 and ROP7 antigens in the course of T. gondii infection, we used T. gondii Pru mutants defective in establishing a chronic infection ([21] and Methods). BALB/c mice infected with mutant parasites, compared to those infected with wild-type parasites, exhibited a CD8⁺ T cell response of altered magnitude at 4 and 8 weeks after infection in the brain (FIG. 6A) Animals infected with the Pru mutant 76-E2, which produced fewer and smaller brain cysts exhibited the most H-2L^{d}/GRA4-specific CD8⁺ T cells at 4 weeks after infection. Interestingly, the ROP7-CD8⁺ T cell response was sharply reduced for this 76-E2 mutant, compared with wild-type Pru at 8 weeks after infection, showing the importance of establishing a chronic infection for development of a T cell response against ROP7. The ROP7-CD8⁺ T cell response peaked early at 4 weeks after infection for the Pru mutant 73F9, which is defective in nuclear trafficking [21]. Even though infection with the 73F9 mutant results in an ˜200-fold reduction in the number of cysts in the brains of mice, compared with infection with the Pru wild type [21], microarray analysis showed that 73F9 developed into bradyzoites faster than wild-type Pru in vitro (P. Davis and D. Roos, personal communication). Pru mutant 9×G4, which has a dramatically reduced lethality in IFN-γ^{-/-} mice, elicits a reduced CD8⁺ T cell response for both GRA4 and ROP7 at 8 weeks after infection. Clearly, the kinetics of parasite-stage conversion and the morphology of the bradyzoite stage influence CD8⁺ T cell specificity.

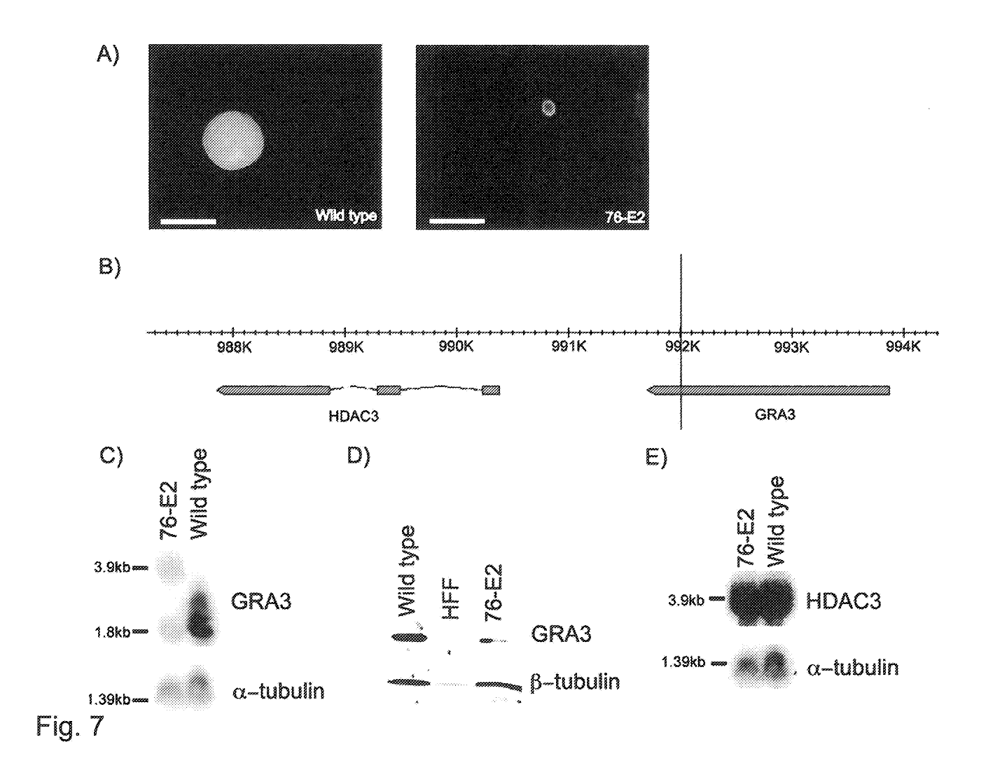

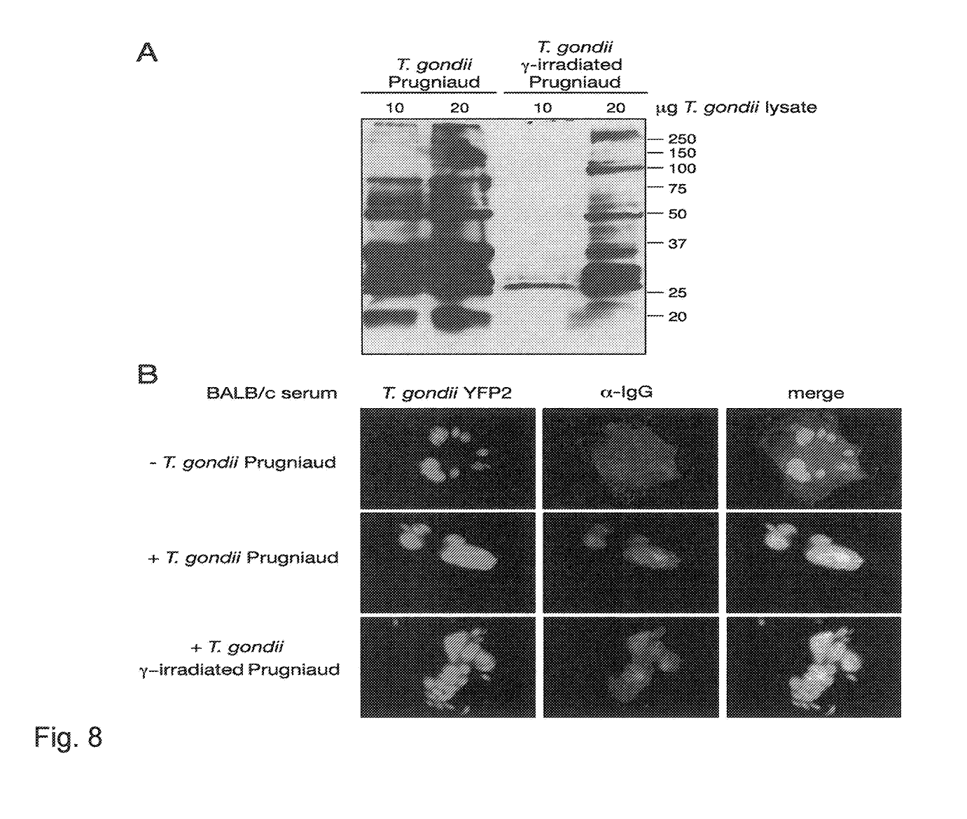

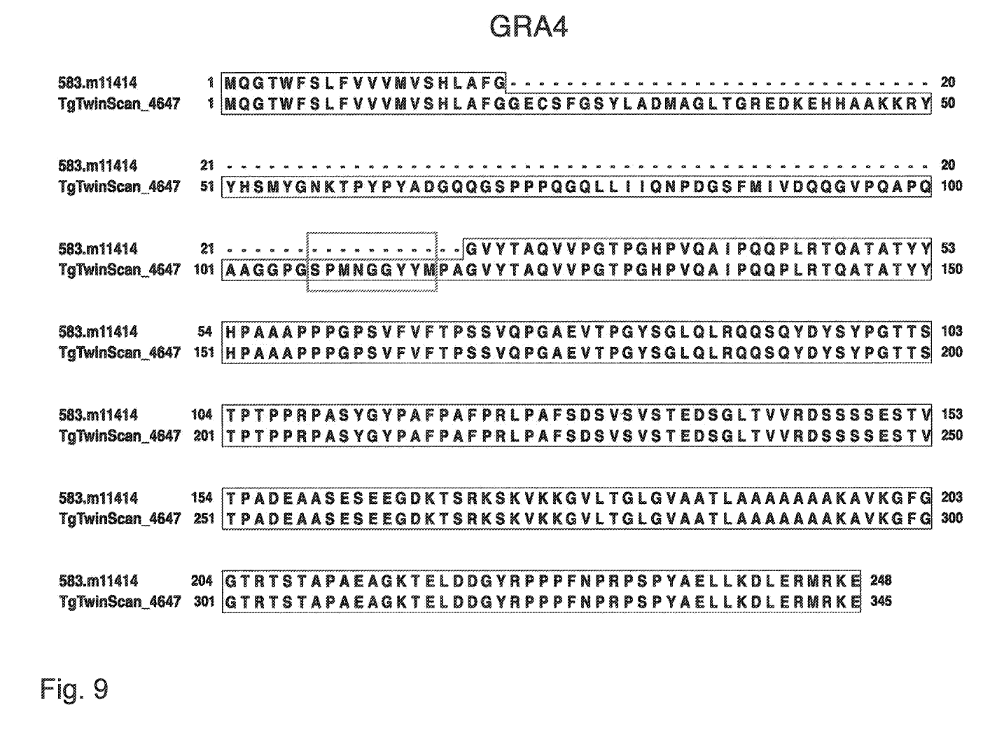

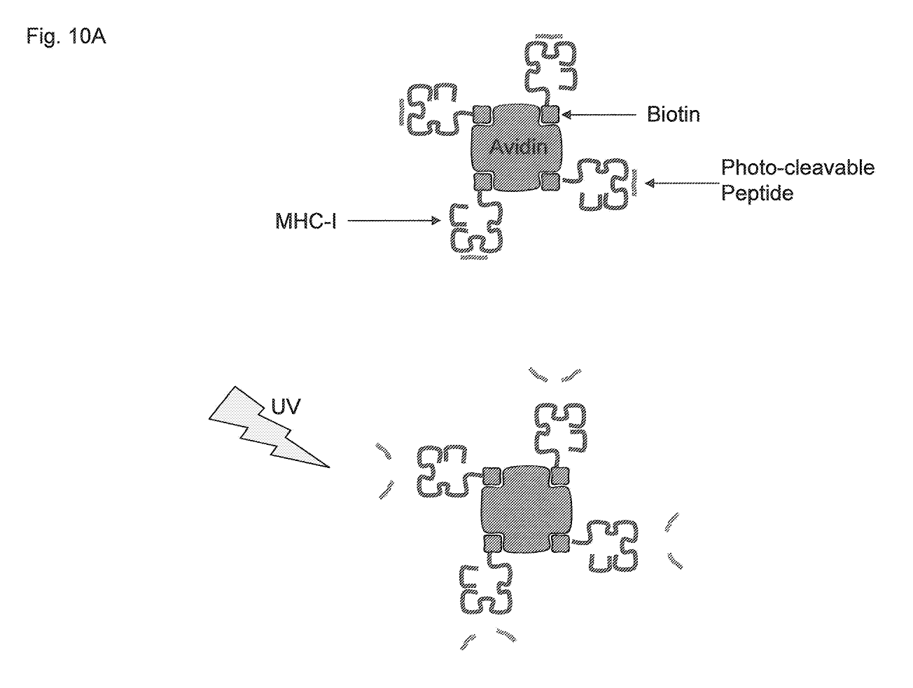

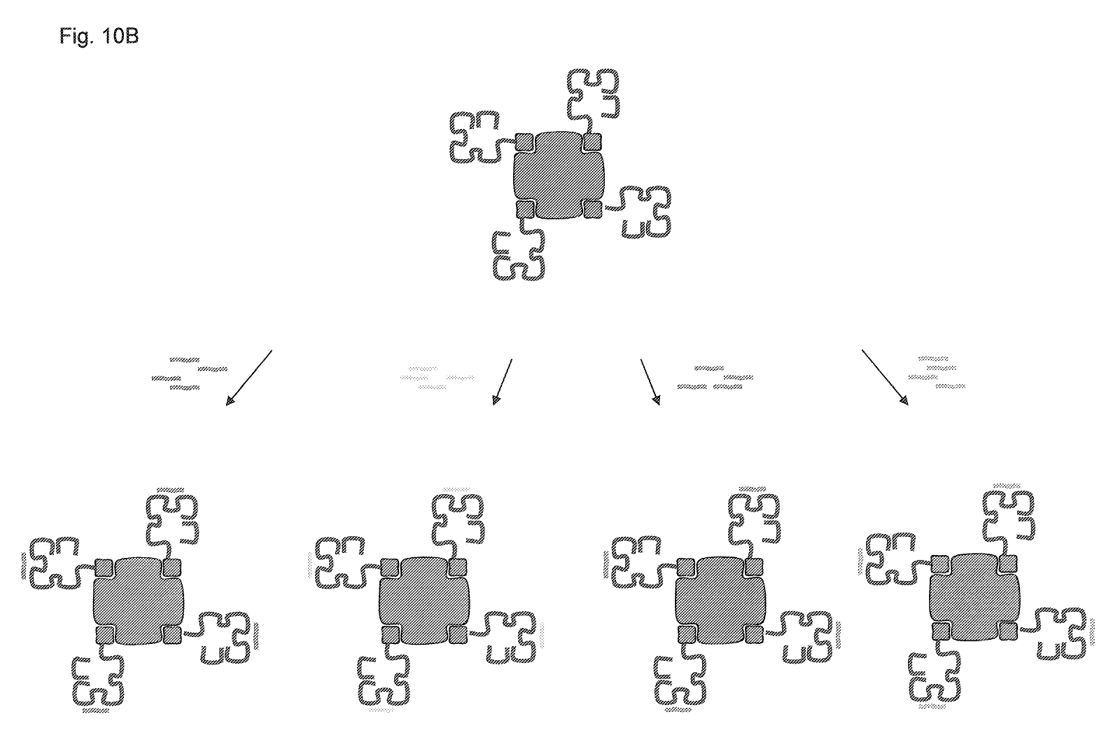

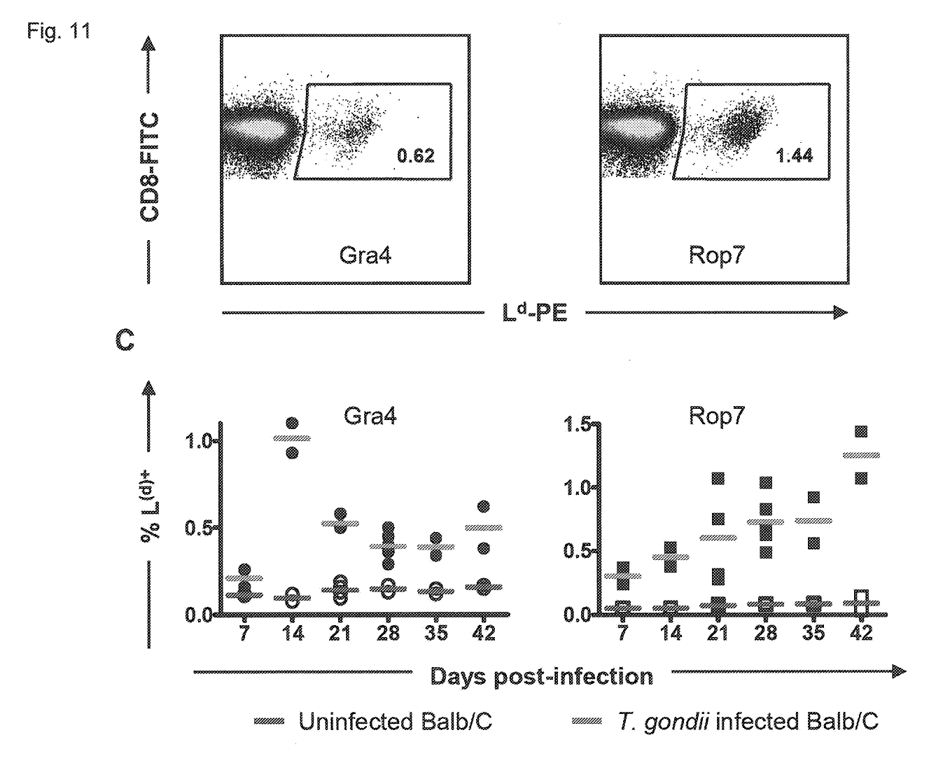

The differences seen for the H-2L^{d}/ROP7-specific T cells prompted us to investigate whether these CD8⁺ T cells could be detected in BALB/c animals infected with replication-deficient T. gondii 6 weeks after infection. BALB/c mice infected with 2×10⁶ γ-irradiated T. gondii Pru tachyzoites failed to elicit an H-2L^{d}/ROP7-specific response in the brain or the spleen (data not shown). We confirmed that all animals were parasite-exposed by checking for serum IgG specific for T. gondii (FIGS. 7B, 7C, and 9). The absence of H-2L^{d}/ROP7 CD8⁺ T cells in this model can be explained by a requirement of the parasite to present the ROP7 antigenic peptide from the bradyzoite stage to CD8⁺ T cells.

Rhoptries are organelles that secrete their contents during invasion of the tachyzoite stage [34], and their function during the bradyzoite stage has not been investigated. We therefore examined the expression levels of ROP7 and GRA4 in relation to each other in the tachyzoite and bradyzoite stage of T. gondii. ME49 bradyzoites were prepared from the brains of infected Swiss-Webster mice and the corresponding ME49 tachyzoites came from serial passage in vitro through fibroblasts (HFFs). GRA4 protein levels were higher during the tachyzoite stage than the bradyzoite stage, whereas expression of ROP7 was comparable between the 2 stages (FIG. 6B).

Discussion

Even though IFN-γ-producing CD8⁺ T cells protect mice against toxoplasmic encephalitis [35, 36], the epitopes that mediate this recognition are unknown. Conventional methods to discover CD8⁺ T cell epitopes are especially difficult and time-consuming for parasitic infections and account for this dearth of information. Therefore, we set up a class I MHC tetramer-based screen to directly visualize parasite-specific CD8⁺ T cells derived from a primary T. gondii infection. We discovered 2 CD8⁺ T cell epitopes derived from the T. gondii proteins GRA4 and ROP7. They are unique to GRA4 and ROP7, and neither epitope is polymorphic for the type I (GT1), type II (ME49) or type III (VEG) strains of T. gondii sequenced to date. Together, these common strains account for 90% of the isolates of T. gondii recovered worldwide. Interestingly, the GRA4 protein induces the proliferation of T lymphocytes from infected animals of an H-2^{d} and H-2^{k} background [37]. Improvements in protein annotation, secretory protein prediction, and stage-specific microarray transcription, as well as mass spectrometry proteome data, available through the Toxoplasma database, should facilitate further selection of proteins for epitope identification. Relaxation of the search criteria to include peptides of 8 and 10 amino acids may increase the yield of CD8⁺ T cell epitopes.

GRA4 is a protein secreted through the apicomplexan-specific dense granules (FIG. 4A) into the parasitophorous vacuole (PV). Here, GRA4 forms a multimeric complex with GRA6 and GRA2 and stably associates with the intravacuolar network [19]. Even though the protein composition of the dense granules within the PV is now known, the function of these proteins is still poorly understood [38]. We found the H-2L^{d}/GRA4-restricted peptide SPMNGGYYM in the annotation by using the TgTwinScan model, but not with the other annotation algorithms (FIG. 9), which is not available in the print version). Rhoptries are the second organelle released during invasion and some ROP proteins are injected directly into the host cell (FIG. 4A) [33]. ROP7 is a rhoptry bulb protein—a member of the ROP2 kinase family—and is injected into the host cell cytoplasm relocalizing to the PV membrane, possibly interacting with the host cell cytoplasm [18, 39]. The epitope IPAAAGRFF in ROP7 maps to its hydrophobic N-terminal region [40]. Because of the sequence similarity of proteins within the ROP2 family, a sequence related to the IPAAAGRFF epitope with a single amino acid difference is present in 2 other rhoptry proteins—IPAAALRFF for both ROP2 and ROP8. However, T cells from T. gondii-infected mice consistently failed to stain with H-2L^{d}/IPAAALRFF tetramers (data not shown).

The frequency of the ROP7-specific CD8⁺ T cells appears somewhat lower than CD8⁺ T cells reactive to the secreted model antigen β-galactosidase [31]. Whether this implies the presence of other antigens important for the CD8⁺ T cell response to this stage of the life cycle of T. gondii or is simply the result of an overexpressed model antigen remains open. Of note, the model antigen-specific T cell population was detected only when β-galactosidase was expressed under a tachyzoite promoter. The identification of 2 endogenous T. gondii antigens that evoke CD8⁺ T cell responses with such different kinetics underscores the importance of knowing the true parasite-derived epitopes. The prominent GRA4-specific CD8⁺ immune response during the first 2 weeks after infection, during the T. gondii tachyzoite stage, correlates with the protein expression data (FIG. 6B). Delivery of the ROP7 antigen during the bradyzoite stage for presentation to CD8⁺ T cells is a definite possibility, as ROP7 is transcribed (M. Matrajt and D. Roos, personal communication) and, according to our data, expressed in both the tachyzoite and bradyzoite stage (FIG. 6B). The failure to detect ROP7-specific CD8⁺ T cells during acute infection might indicate the presence of yet another population of CD8⁺ T cells that contributes to the early response. Moreover, the secreted protein pool in tachyzoites is large and complex, with other proteins presumably dominating the response. In bradyzoites, the mixture of secreted proteins is less diverse, and the exchange with the host cell is dramatically reduced. ROP proteins are required to assure host cell survival [34]. Because bradyzoites have an extended life span, the ROP proteins might have an as yet underappreciated role in bradyzoites, and if so, they are likely the most abundantly secreted factors.

The pathway for delivery of antigens into the MHC class I presentation pathway for proteins expressed into the parasitophorous vacuole remains elusive [41]. Both GRA4 and ROP7 are transcribed equally in Pru tachyzoites and ME49 cysts, as judged by microarrays (A. Bahl and D. Roos, personal communication). However, we observed altered protein levels for GRA4 in the 2 parasitic stages, and it is tempting to speculate that escape from the PV for GRA4 is differentially regulated during the tachyzoite stage as opposed to the bradyzoite stage. Indeed, GRA4 may be limited or not secreted during the bradyzoite stage and is not part of the cyst wall [42, 43]. ROP7, on the other hand, is most likely associated with the PV membrane, possibly in the host cell cytoplasm [3, 18]. Regardless of the level and timing of expression, ROP7 and GRA4 must access the class I processing machinery. Intracerebral CD8⁺ T cells infiltrate from the acute phase of T. gondii infection in response to a transgenically expressed tachyzoite-stage antigen; they persist and finally are slowly eliminated by apoptosis [44]. Unknown expression levels and localization of the transgenic antigen chosen may account for the seemingly different behavior we see for the H-2L^{d}/ROP7-specific intracerebral T cells. Selective traffic of antigen-specific CD8⁺ T cells into the brain occurs in vivo and is dependent on expression of class I MHC by cerebral endothelium and the presence of the cognate antigen [45]. Once in the brain, the CD8⁺ T cells can undergo additional proliferation [46]. With the identification of 2 stage-specific endogenous T. gondii CD8⁺ epitopes, these questions now become tractable without the need for genetically engineered parasites [47].

Underperformance of the immune system causes recrudescence of T. gondii. Continuous surveillance by CD8⁺ T cells likely keeps T. gondii under control, and candidate CD8⁺ T cells capable of such surveillance include those that recognize ROP7, expressed even at the bradyzoite stage. Our results represent an important step toward a more complete characterization of the immune response to T. gondii. The possible identification of epitopes that afford protection against the outgrowth of parasites in cysts may facilitate the development of strategies to vaccinate against or otherwise control this widespread and clinically important pathogen.

Materials and Methods

Antibodies and parasite strains. J. F. Dubremetz provided ROP7 antibody T₄3H₁ [18], D. Sibley provided the GRA4 antibody [19], J. Saeij provided the SAG2Y antibody [20], and J. Gaertig provided the 12G10 anti-tubulin antibody. T. gondii Pru AHXGPRT and ME49 tachyzoites were propagated in human foreskin fibroblast (HFF) monolayers grown in Dulbecco's modified Eagle medium containing 10% fetal calf serum and penicillin-streptomycin. The 73F9 and 9×G4 mutants were previously isolated [21].

Isolation and characterization of the 76-E2 mutant. Mutant 76-E2 was identified in a secondary screen of a signature-tagged insertional mutant library [21] as reduced in bradyzoite development within tissue culture of HFF cells, It was 1.5-fold reduced in complete Dolichos biflorus cyst wall formation and 14-fold reduced in BAG1 expression, compared with both the wild-type and E2 parental parasites. In vivo brain cyst counts for 76-E2 after 4 weeks in CBA/J mice were 10-fold to 40-fold lower than counts for the wild type (data not shown). However, the most striking phenotype of the 76-E2 mutant in mice was the absence of larger cysts (25-50 μm) that are commonly seen in mice infected with wild type Pru (FIG. 7A). The E2 parental strain was indistinguishable from wild-type parasites.

The insertion site of the pLK47 plasmid in the 76-E2 mutant was in the 3′UTR of the Dense Granule 3 gene (GRA3; FIG. 7B). This insertion disrupted some but not all of the GRA3 transcripts (FIG. 7C) and reduced the GRA3 protein expression level (FIG. 7D). Complementation of the 76-E2 mutant with the full GRA3 genomic locus did not restore the bradyzoite development phenotype (data not shown). Deletion of the entire GRA3 ORF was unsuccessful after multiple attempts; however, disruption of the GRA3 protein alone by removal of the GRA3 promoter and start codon resulted in >80% efficiency of homologous recombination at the locus. Strains lacking GRA3 did not recreate the 76-E2 mutant phenotypes observed in vitro and in vivo. This indicated that the GRA3 protein was not involved in the development phenotypes observed [50]. Immediately downstream of the GRA3 locus is a histone deacetylase 3 (HDAC3) that has been shown to be important for the timed expression of genes during development [51]. To determine whether the insertion in 76-E2 disrupted expression of HDAC3, a Northern blot analysis was preformed with tachyzoite RNA using a probe to the HDAC3 ORF (FIG. 7E). Expression of HDAC3 does not appear to be disrupted in 76-E2.

To determine whether the insertion site in the 76-E2 mutant was the cause of the bradyzoite development phenotype, the exact insertion site was targeted by homologous recombination. Disruption of the exact insertion site with a positive selectable marker occurred in 100% of the clones from 2 independent electroporations; however, the in vitro bradyzoite development defect was not recapitulated (data not shown). Although it is likely that an electroporation-induced mutation occurred in the genome of the 76-E2 mutant to cause the observed defect, it is also possible that the size of the insertion is responsible for the observed phenotype. The pLK47 plasmid inserted in tandem at least 4 times, creating an insertion >20 kb that could disrupt the chromatin structure in the local area. This may cause expression defects in neighboring genes further up or downstream. It is intriguing that homologous recombination was so high at the locus for certain disruptions but not for a larger deletion at this locus. Future microarray analysis of the 76-E2 mutant will allow us to determine the affected genes.

Characterization of the 9×G4 mutant. The mutants uncovered in the modified signature-tagged mutagenesis screen were analyzed for their lethality to IFN-γ^{-/-} mice. These mice (originally purchased from the JAX laboratories and bred at the University of Wisconsin) were infected with 25 tachyzoites, syringe-lysed from HFF cells. Immediately after infection, parasites were analyzed by plaque assay to ensure accurate enumeration and viability. Two mice were infected for each mutant or wild-type Pru parasite, and the experiments were repeated at least twice. The 9×G4 mutant had dramatically reduced lethality, with half of the infected IFN-γ^{-/-} mice surviving and the life span of the other half being extended to 14 and 15 days. Wild-type Pru was lethal to IFN-γ^{-/-} mice as had been seen before [52], with all mice succumbing to infection by 9 days after infection. The 2 IFN-γ^{-/-} mice that survived infection with 9×G4 were sacrificed at 22 days after infection; their serum showed reactivity to T. gondii and their brains contained a few small bradyzoite cysts.

Experimental animals and T. gondii infection. All animal protocols were approved by the Massachusetts Institute of Technology (MIT) Committee on Animal Care. Swiss-Webster and BALB/c mice were obtained from Taconic. Forty cysts of the T. gondii ME49 strain (a gift from G. Yap) were isolated from the brain homogenate of an infected Swiss-Webster animal and injected intraperitoneally into BALB/c animals for future analysis of the T cell response. Alternatively, BALB/c mice were infected with 5000 Prugniaud tachyzoites or 2×10⁶ γ-irradiated Prugniaud tachyzoites (15 kRad).

Composition of the Toxoplasma secretome gene list. ToxoDB version 3.0 [47] was used to extract a list of TgTwinScan gene predictions with a presumed signal peptide. Genes with a predicted GPI anchor were removed from the list. In addition, 66 plastid targeted proteins, which also contain a signal peptide, were removed from the list. Genes with an expected plastid localization were identified by BLAST homology comparison (E+1 cutoff) against the complete catalogue of plastid targeted proteins in Plasmodium (PlasmoDB). The remaining 467 candidates where checked manually for SAGE and EST hits: genes with bradyzoite-only ESTs [31] and genes with no expression data were removed from the list, unless alignments with putative microneme, rhoptry, or dense granule proteins from other apicomplexans were apparent. Furthermore, predicted ORFs of <100 aa were ignored. The condensed list consisted of 57 and was supplemented with genes whose products show experimentally verified microneme [48] and rhoptry localization [49]. The final list consisted of 73 T. gondii proteins (see U.S. Ser. No. U61/077,807 and U.S. Ser. No. 61/077,835).

H-2L^{d} epitope prediction. The 73 selected open reading frames (ORFs) were analyzed by use of the Web-based predictive algorithms BIMAS [22], RANKPEP [23], and SYFPEITHI [24] and predicted 9 amino acid residue epitopes that scored higher then 150, 92, and 21, respectively, were incorporated into the screen. Double hits were removed, yielding 246 unique nonameric sequences.

Peptide synthesis. Conditional ligands p29-P6* to p29-P8* were constructed manually, using 9-flourenylmethloxycarbonyl-based solid-phase peptide synthesis. The MIT Center for Cancer Research biopolymers facility (Cambridge, Mass.) synthesized the peptides used for screening.

Protein expression and purification. Recombinant expression of murine β₂m, as well as the luminal portion of the H-2L^{d} heavy chain with a C-terminal BirA recognition sequence (plasmid gift of J. D. Altman), was accomplished by following established protocols. Refolding of the MHC complex with conditional ligands p29-P6* to p29-P8* was followed by biotinylation, size-exclusion chromatography (S75) [25], and assembled monomers were stored at −80° C. Class I MHC tetramers [26] were produced by addition of streptavidin-phycoerythrin (Invitrogen) to monomer at a final molar ratio of 4:1, respectively, and peptide exchange was effected by irradiation at 365 nm (Stratalinker 2400 UV), as described elsewhere [16].

Cell preparation and tetramer staining. The peritoneal cavity of BALB/c mice was lavaged, and splenocytes and mononucleated cells from the brain were prepared. For screening, CD8⁺ T cells were isolated with a Milteny Biotech kit. To purify brain T cells, the mice were perfused intracardially with PBS and the brain homogenized and passed over a 35% Percoll solution, followed by a 70%/35% Percoll gradient. Cell suspensions were treated with ethidium monoazide under exclusion of light, and washed and irradiated with incandescent light. The cells were incubated for 45 min in 96-well plates (˜1×10⁵ cells at 50 μL/well) with freshly prepared H-2L^{d} tetramer and fluorescein isothiocyanate-conjugated anti-CD8 mAb (Becton Dickinson) followed by fixing with 4% formaldehyde, and they were then analyzed by flow cytometry.

Cyst purification. Brains from ME49-infected Swiss-Webster mice were homogenized in 1% Tween 20 in PBS. The homogenate was passed over a 90%/30% Percoll gradient and centrifuged. The 90%/30% interface and half of the 30% layer were collected and repeatedly washed with PBS.

In vitro intracellular IFN-γ detection. Splenocytes from Pru- or ME49-infected BALB/c mice were seeded at 4×10⁶ cells per well and restimulated overnight with 10 μg/mL of peptide. Cells were treated for 4 h with 10 μg/mL brefeldin A, then labeled with tetramer and anti-CD8 mAb as described above and stained with anti-IFN-γ mAb by using the BD Cytofix/Cytoperm kit (Becton Dickinson).

Western blot analysis. ME49 tachyzoites, and bradyzoites harvested from mouse brain were lysed in PBS by freeze-thawing. We then analyzed 0.5, 2, 5 and 10 μg of the tachyzoite and bradyzoite lysates by SDS-PAGE. Immunoblot analysis was performed with ROP7 antibodies, GRA4 antibodies, SAG2Y antibodies, or tubulin antibodies at dilutions of 1:1000, 1:2000, 1:2000, and 1:1000, respectively.

### References for Example 2

1. Montoya J G, Liesenfeld O. Toxoplasmosis. Lancet 2004; 363:1965-76.

2. Dubey J P, Lindsay D S, Speer C A. Structures of Toxoplasma gondii tachyzoites, bradyzoites, and sporozoites and biology and development of tissue cysts. Clin Microbiol Rev 1998; 11:267-99.

3. Laliberte J, Carruthers V B. Host cell manipulation by the human pathogen Toxoplasma gondii. Cell Mol Life Sci 2008; 65:1900-15.

4. Weiss L M, Kim K. The development and biology of bradyzoites of Toxoplasma gondii. Front Biosci 2000; 5:D391-405.

5. Denkers E Y, Gazzinelli R T. Regulation and function of T-cell-mediated immunity during Toxoplasma gondii infection. Clin Microbiol Rev 1998; 11:569-88.

6. McLeod R, Skamene E, Brown C R, Eisenhauer P B, Mack D G. Genetic regulation of early survival and cyst number after peroral Toxoplasma gondii infection of A×B/B×A recombinant inbred and B10 congenic mice. J Immunol 1989; 143:3031-4.

7. Johnson J J, Roberts C W, Pope C, et al. In vitro correlates of Ld-restricted resistance to toxoplasmic encephalitis and their critical dependence on parasite strain. J Immunol 2002; 169:966-73.

8. Suzuki Y, Orellana M A, Schreiber R D, Remington J S. Interferon-gamma: the major mediator of resistance against Toxoplasma gondii. Science 1988; 240:516-8.

9. Suzuki Y, Conley F K, Remington J S. Importance of endogenous IFN-gamma for prevention of toxoplasmic encephalitis in mice. J Immunol 1989; 143:2045-50.

10. Gazzinelli R, Xu Y, Hieny S, Cheever A, Sher A. Simultaneous depletion of CD4+ and CD8+ T lymphocytes is required to reactivate chronic infection with Toxoplasma gondii. J Immunol 1992; 149:175-80.

11. Khan I A, Ely K H, Kasper L H. Antigen-specific CD8+ T cell clone protects against acute Toxoplasma gondii infection in mice. J Immunol 1994; 152:1856-60.

12. Nielsen H V, Lauemoller S L, Christiansen L, Buus S, Fomsgaard A, Petersen E. Complete protection against lethal Toxoplasma gondii infection in mice immunized with a plasmid encoding the SAG1 gene. Infect Immun 1999; 67:6358-63.

13. Desolme B, Mevelec M N, Buzoni-Gatel D, Bout D. Induction of protective immunity against toxoplasmosis in mice by DNA immunization with a plasmid encoding Toxoplasma gondii GRA4 gene. Vaccine 2000; 18:2512-21.

14. Caetano B C, Bruna-Romero O, Fux B, Mendes E A, Penido M L, Gazzinelli R T. Vaccination with replication-deficient recombinant adenoviruses encoding the main surface antigens of Toxoplasma gondii induces immune response and protection against infection in mice. Hum Gene Ther 2006; 17:415-26.

15. Toebes M, Coccoris M, Bins A, et al. Design and use of conditional MHC class I ligands. Nature Medicine 2006; 12:246-51.

16. Grotenbreg G M, Roan N R, Guillen E, et al. Discovery of CD8+ T cell epitopes in Chlamydia trachomatis infection through use of caged class I MHC tetramers. Proc Natl Acad Sci USA 2008; 105:3831-6.

17. Bakker A H, Hoppes R, Linnemann C, et al. Conditional MHC class I ligands and peptide exchange technology for the human MHC gene products HLA-A1, -A3, -A11, and -B7. Proc Natl Acad Sci USA 2008; 105:3825-30.

18. Hajj H E, Lebrun M, Fourmaux M N, Vial H, Dubremetz J F. Characterization, biosynthesis and fate of ROP7, a ROP2 related rhoptry protein of Toxoplasma gondii. Mol Biochem Parasitol 2006; 146:98-100.

19. Labruyere E, Lingnau M, Mercier C, Sibley L D. Differential membrane targeting of the secretory proteins GRA4 and GRA6 within the parasitophorous vacuole formed by Toxoplasma gondii. Mol Biochem Parasitol 1999; 102:311-24.

20. Saeij J P, Arrizabalaga G, Boothroyd J C. A cluster of four surface antigen genes specifically expressed in bradyzoites, SAG2CDXY, plays an important role in Toxoplasma gondii persistence. Infect Immun 2008; 76:2402-10.

21. Frankel M B, Mordue D G, Knoll L J. Discovery of parasite virulence genes reveals a unique regulator of chromosome condensation 1 ortholog critical for efficient nuclear trafficking, Proc Natl Acad Sci USA 2007; 104:10181-6.

22. Parker K C, Bednarek M A, Coligan J E. Scheme for ranking potential HLA-A2 binding peptides based on independent binding of individual peptide side-chains. J Immunol 1994; 152:163-75.

23. Reche P A, Glutting J P, Zhang H, Reinherz E L. Enhancement to the RANKPEP resource for the prediction of peptide binding to MHC molecules using profiles. Immunogenetics 2004; 56:405-19.

24. Rammensee H G, Bachmann J, Emmerich N P N, Bachor O A, Stevanovic S. SYFPEITHI: database for MHC ligands and peptide motifs. Immunogenetics 1999; 50:213-9.

25. Garboczi D, Hung D, Wiley D. HLA-A2-peptide complexes: refolding and crystallization of molecules expressed in Escherichia coli and complexed with single antigenic peptides. Proc Natl Acad Sci USA 1992; 89:3429-33.

26. Altman J D, Moss P A H, Goulder P J R, et al. Phenotypic analysis of antigen-specific T lymphocytes. Science 1996; 274:94-96.

27. Grotenbreg G M, Nicholson M J, Fowler K D, et al. Empty class II major histocompatibility complex created by peptide photolysis establishes the role of DM in peptide association. J Biol Chem 2007; 282:21425-36.

28. Corr M, Boyd L F, Frankel S R, Kozlowski S, Padlan E A, Margulies D H. Endogenous peptides of a soluble major histocompatibility complex class I molecule, H-2Lds: sequence motif, quantitative binding, and molecular modeling of the complex. J Exp Med 1992; 176:1681-92.

29. Balendiran G K, Solheim J C, Young A C, Hansen T H, Nathenson S G, Sacchettini J C. The three-dimensional structure of an H-2Ld-peptide complex explains the unique interaction of Ld with beta-2 microglobulin and peptide. Proc Natl Acad Sci USA 1997; 94:6880-5.

30. Chen J, Eisen H N, Kranz D M. A model T-cell receptor system for studying memory T-cell development. Microbes Infect 2003; 5:233-240.

31. Kwok L Y, Lutjen S, Soltek S, et al. The induction and kinetics of antigen-specific CD8 T cells are defined by the stage specificity and compartmentalization of the antigen in murine toxoplasmosis. J Immunol 2003; 170:1949-57.

32. Kissinger J C, Gajria B, Li L, Paulsen I T, Roos D S. ToxoDB: accessing the Toxoplasma gondii genome. Nucleic Acids Res 2003; 31:234-6.

33. Gubbels M J, Li C, Striepen B. High-throughput growth assay for Toxoplasma gondii using yellow fluorescent protein. Antimicrob Agents Chemother 2003; 47:309-16.

34. Boothroyd J C, Dubremetz J F. Kiss and spit: the dual roles of Toxoplasma rhoptries. Nat Rev Microbiol 2008; 6:79-88,

35. Parker S J, Roberts C W, Alexander J. CD8+ T cells are the major lymphocyte subpopulation involved in the protective immune response to Toxoplasma gondii in mice. Clin Exp Immunol 1991; 84:207-12.

36. Khan I A, Green W R, Kasper L H, Green K A, Schwartzman J D. Immune CD8(+) T cells prevent reactivation of Toxoplasma gondii infection in the immunocompromised host. Infect Immun 1999; 67:5869-76.

37. Chardes T, Velge-Roussel F, Mevelec P, Mevelec M N, Buzoni-Gatel D, Bout D. Mucosal and systemic cellular immune responses induced by Toxoplasma gondii antigens in cyst orally infected mice. Immunology 1993; 78:421-9.

38. Mercier C, Adjogble K D, Daubener W, Delauw M F. Dense granules: are they key organelles to help understand the parasitophorous vacuole of all apicomplexa parasites? Int J Parasitol 2005; 35:829-49.

39. El Hajj H, Lebrun M, Fourmaux M N, Vial H, Dubremetz J F. Inverted topology of the Toxoplasma gondii ROP5 rhoptry protein provides new insights into the association of the ROP2 protein family with the parasitophorous vacuole membrane. Cell Microbiol 2007; 9:54-64.

40. El Hajj H, Demey E, Poncet J, et al. The ROP2 family of Toxoplasma gondii rhoptry proteins: proteomic and genomic characterization and molecular modeling. Proteomics 2006; 6:5773-84.

41. Gubbels M J, Striepen B, Shastri N, Turkoz M, Robey E A. Class I major histocompatibility complex presentation of antigens that escape from the parasitophorous vacuole of Toxoplasma gondii. Infect Immun 2005; 73:703-11.

42. Ferguson D J, Cesbron-Delauw M F, Dubremetz J F, Sibley L D, Joiner K A, Wright S. The expression and distribution of dense granule proteins in the enteric (Coccidian) forms of Toxoplasma gondii in the small intestine of the cat. Exp Parasitol 1999; 91:203-11.

43. Ferguson D J. Use of molecular and ultrastructural markers to evaluate stage conversion of Toxoplasma gondii in both the intermediate and definitive host. Int J Parasitol 2004; 34:347-60.

44. Schluter D, Meyer T, Kwok L Y, et al. Phenotype and regulation of persistent intracerebral T cells in murine Toxoplasma encephalitis. J Immunol 2002; 169:315-22.

45. Galea I, Bernardes-Silva M, Forse P A, van Rooijen N, Liblau R S, Perry V H. An antigen-specific pathway for CD8 T cells across the blood-brain barrier. J Exp Med 2007; 204:2023-30.

46. Ling C, Sandor M, Suresh M, Fabry Z. Traumatic injury and the presence of antigen differentially contribute to T-cell recruitment in the CNS. J Neurosci 2006; 26:731-41.

47. Dzierszinski F S, Hunter C A. Advances in the use of genetically engineered parasites to study immunity to Toxoplasma gondii. Parasite Immunol 2008; 30:235-44.

48. Zhou X W, Kafsack B F, Cole R N, Beckett P, Shen R F, Carruthers V B. The opportunistic pathogen Toxoplasma gondii deploys a diverse legion of invasion and survival proteins. J Biol Chem 2005; 280:34233-44.

49. Bradley P J, Ward C, Cheng S J, et al. Proteomic analysis of rhoptry organelles reveals many novel constituents for host-parasite interactions in Toxoplasma gondii. J Biol Chem 2005; 280:34245-58.

50. Craver M P, Knoll L J. Increased efficiency of homologous recombination in Toxoplasma gondii dense granule protein 3 demonstrates that GRA3 is not necessary in cell culture but does contribute to virulence. Mol Biochem Parasitol 2007; 153:149-57.

51. Saksouk N, Bhatti M M, Kieffer S, et al. Histone-modifying complexes regulate gene expression pertinent to the differentiation of the protozoan parasite Toxoplasma gondii. Mol Cell Biol 2005; 25:10301-14.

52. Scharton-Kersten T M, Wynn T A, Denkers E Y, et al. In the absence of endogenous IFN-gamma, mice develop unimpaired IL-12 responses to Toxoplasma gondii while failing to control acute infection. J Immunol 1996; 157:4045-54.

### Example 3

### Generation of Monoclonal Mice with Pre-Defined Specificity Against Toxoplasma gondii Epitopes via Somatic Cell Nuclear Transfer

SCNT was used to generate monoclonal mice using T cells specific for a T. gondii epitope of interest as nuclear donors. As described in Example 2, T. gondii specific H-2Ld-restricted T cell epitopes were identified, one from dense granule protein GRA4 and the other from rhoptry protein ROP7. A third epitope, denoted Kb-A4, was identified using Kb-tetramers. It has the sequence SVLAFRRL from the protein designated >TgTwinScan_{—}1327 identified previously (Carruthers, et al., J. Biol. Chem., 280(40): 34233-34244 (2005)). To isolate T cells specific for a particular epitope, B6CF1 male mice were infected with T. gondii ME49 and splenocytes for SCNT were prepared on day 10 p.i. (tgd057 and Gra4) or day 18 p.i. (Rop7) as described previously (15). To isolate splenocytes, the spleen of a T. gondii-infected mouse was disrupted between two frosted glass slides. The erythrocytes were lysed with ammonium chloride and the remaining cells washed two times with PBS and passed through a 70 μm cell strainer. Subsequently, the cells were distributed over 96-well plates (˜1×10⁵ cells in 50 μL/well) that contained saturating amounts of freshly prepared MHC tetramer containing the epitope of interest and FITC-conjugated anti-CD8 mAb (Becton Dickinson) and were incubated for 45 min. The cells were then washed once with PBS and taken up in PBS containing 1 μM propidium iodide (PI). T cells were sorted gating on PI-negative and the CD8/MHC-tetramer double positive cells using a FACSAria flow cytometer. Sorted cells were kept in RPMI 1640 medium supplemented with 10% fetal calf serum, penicillin (100 U/ml), streptomycin (100μg/ml) and 2 mM glutamine, and used for nuclear transfer within two hours after sorting. Experiments in which cells were returned to culture and used 1 to 7 days later did not yield SCNT-ES cells or live offspring in this initial work.

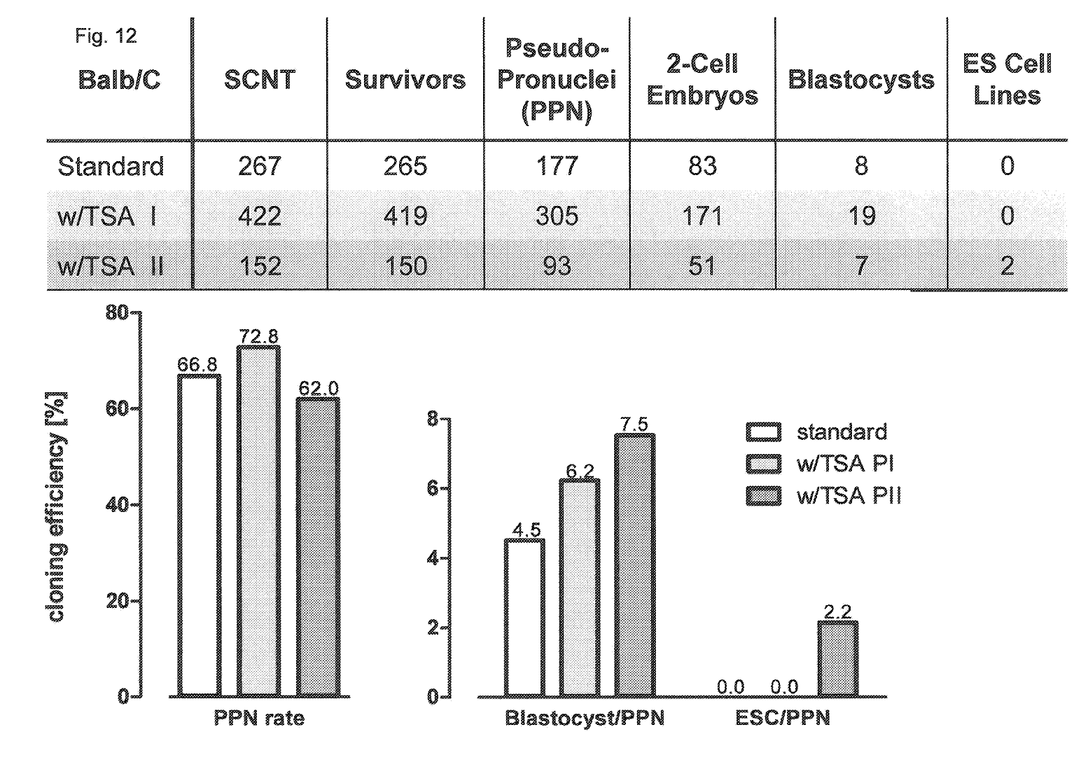

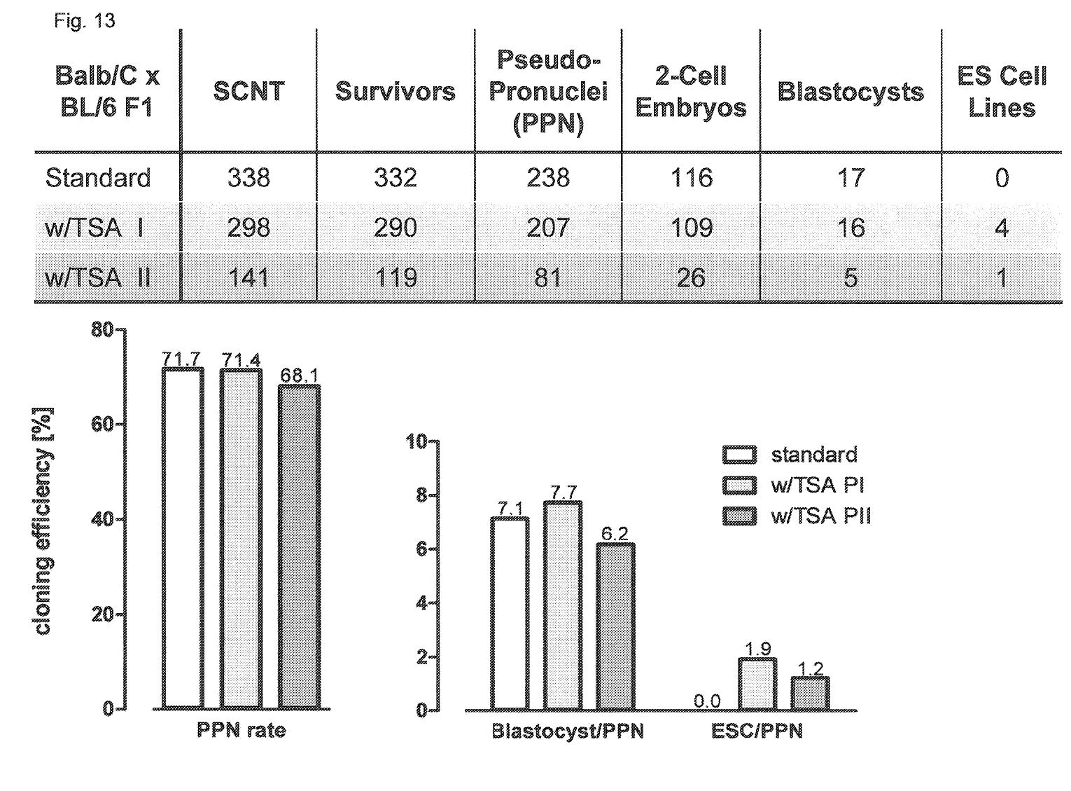

Somatic cell nuclear transfer was performed using standard techniques in which the donor nucleus was introduced into an enucleated oocyte using piezo-actuated micromanipulation (see, e.g., Hochedlinger, K. and Jaenisch, R., Nature, 415(6875): 1035-8 (2002); Kishigami S., et al., Nat Protoc., 1(1):125-38 (2006)). In some experiments, medium was supplemented with 5 nM trichostatin A (TSA) for 6 hours during oocyte activation (Protocol I). In other experiments, TSA was present for 6 hours during activation and for an additional 4 hours after activation, for a total of 10 hours (Protocol II) (Kishigami, S., et al., Biochem Biophys Res Commun, 340(1): 183-9 (2006); Rybouchkin, A., Y. Kato, and Y. Tsunoda, Biol Reprod, 74(6): p. 1083-9 (2006), ref. 17). Surviving embryos were maintained in culture under conditions suitable for development into fertilized blastocysts. ES cells were isolated from the inner cell mass of surviving blastocysts, and ES cell lines were established. A total of seven SCNT-derived ES cell lines were derived from CD8+ T cells specific for three different peptide-MHC complexes. Results obtained using Balb/c mice as nuclear donors are summarized in FIG. 12. Results obtained using Balb/c×Black 6 (B6) F1 mice as nuclear donors are summarized in FIG. 13. These results show that the technique can be applied using inbred or outbred mice as donors. The recipient oocytes were obtained from Black6×DBA2 F2 mice in all experiments.

In summary, we used CD8⁺ T cells specific for L^{d}-Tg-Rop7¹⁶¹⁻¹⁶⁹, L^{d}-Tg-Gra4¹⁰⁷⁻¹¹⁵, and K^{b}-Tg-tgd057⁵⁹⁻⁶⁶ on the BL/6×Balb/c F1 (B6CF1) background as donor cells for SCNT. Employing Trichostatin A (TSA) to inhibit histone deacetylases, we tested three different conditions (no TSA, 6 h TSA, and 10 h TSA) aimed at improving nuclear reprogramming (FIGS. 5 and 6). We generated SCNT-blastocysts with an overall efficiency of 7.2% per pseudo-pronucleus (PPN) in all three conditions, and obtained ES cells when using TSA treatment for 6 h or 10 h but not when using standard conditions. As a control, fertilized blastocysts on the B6CF1 background yielded more than 90% ES cells per blastocyst with and without TSA treatment (data not shown). When CD8⁺ T cells specific for L^{d}-Tg-Rop7¹⁶¹⁻¹⁶⁹ from pure Balb/c background were used as donor cells, we successfully derived SCNT-ES cells with similar efficiency, but only after TSA treatment for 10 h, confirming recent reports that TSA treatment indeed facilitates cloning of inbred mice (17, 18).

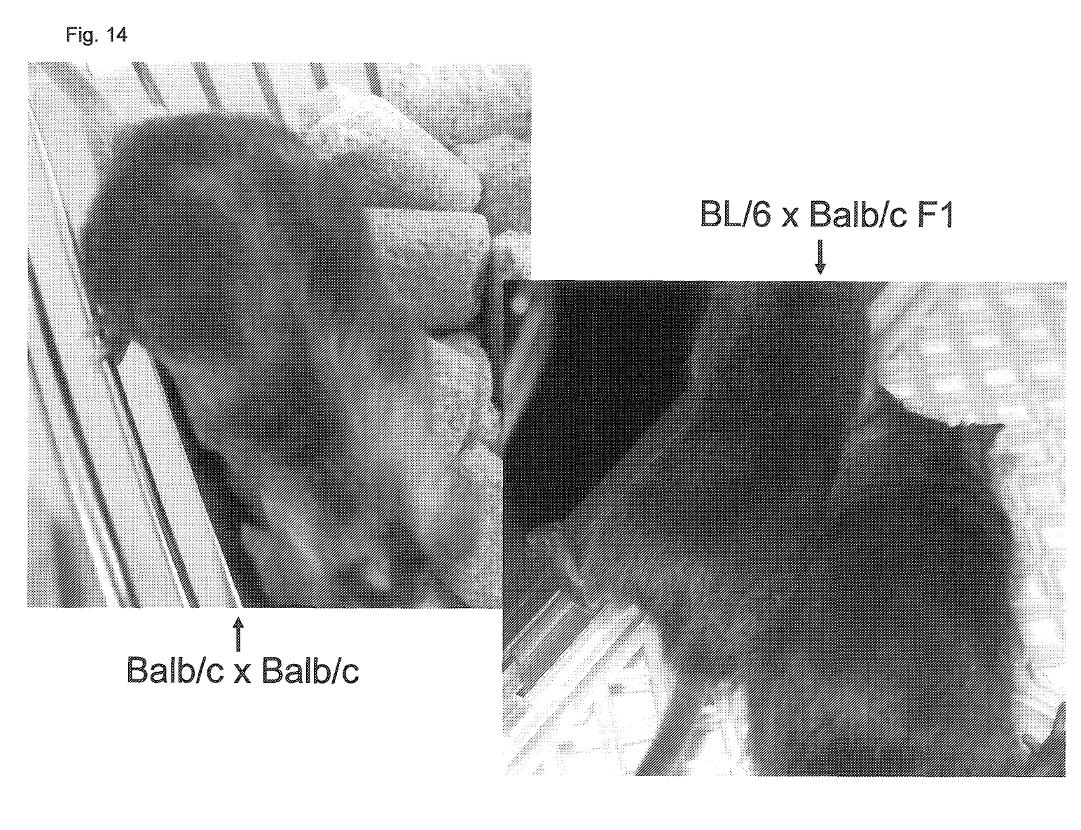

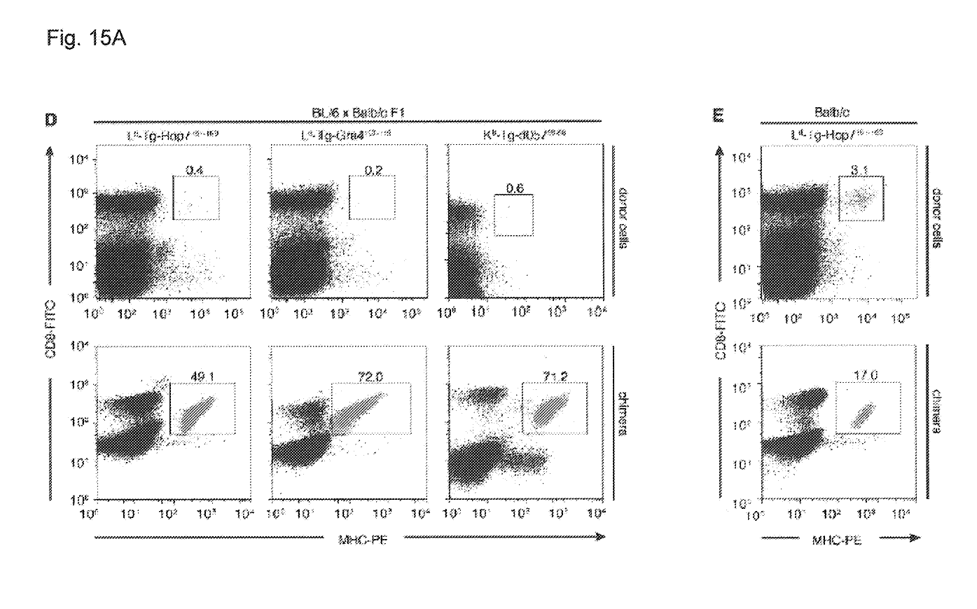

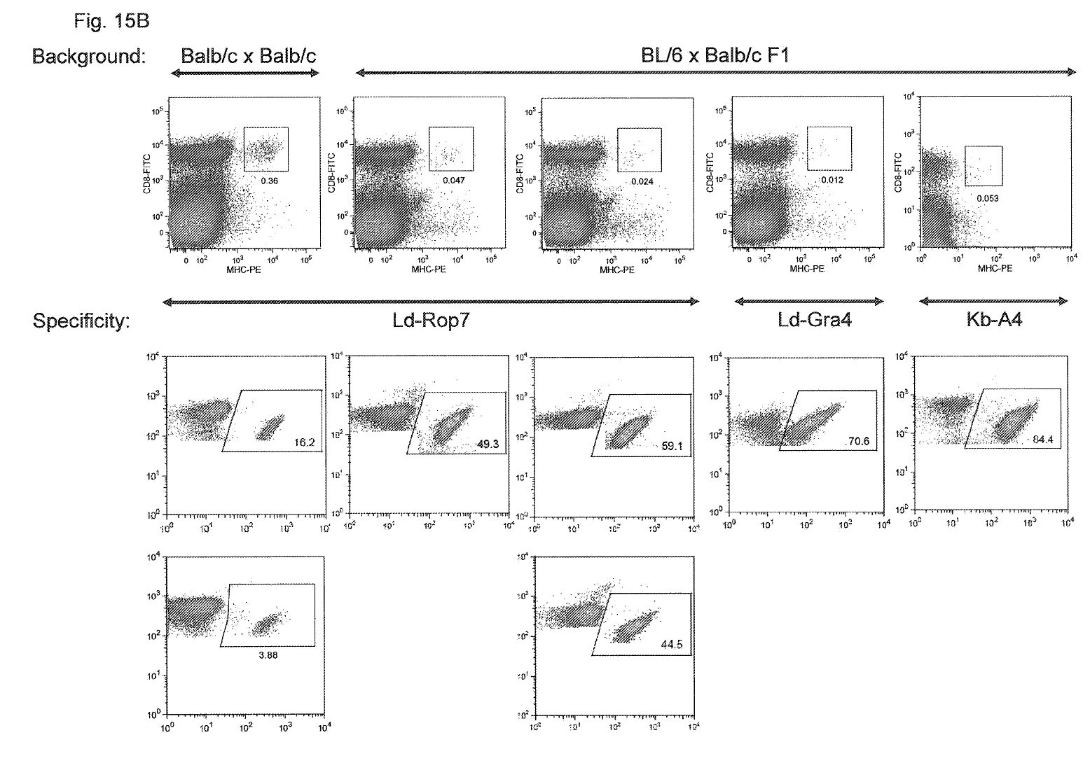

The SCNT-derived ES cell lines were used to generate chimeras by injecting them into blastocysts, which were transferred to pseudopregnant female mice. Chimeric mice derived from this process are shown in FIG. 14. We generated chimeric mice using the five SCNT-ESC lines from the B6CF1 background, all of which showed pronounced populations of tetramer-positive CD8⁺ T cells in the absence of any antigen exposure (FIGS. 15A and 15B). Chimeric mice derived from the two SCNT-ESC lines on a pure Balb/c background also yielded CD8+ T cells of correct specificity (FIGS. 15A and 15B), We showed that all seven SCNT-derived ES cells contributed to the hematopoietic system and accordingly yielded CD8+ T cells that recognize the corresponding peptide-MHC complexes in the absence of immunization (see FIG. 15, where “background” represents T. gondii infected wild type mice). In summary, we thus cloned T cells of desired specificity in seven out of seven cases, a rate that not only depends on TSA treatment, but also on stringent sorting criteria to obtain the necessary donor cells. Because such mice, generated via somatic cell nuclear transfer of T cells (or B cells) with pre-selected specificity, represent a new type of mouse model, we refer to these animals as transnuclear (TN) mice or monoclonal mice.

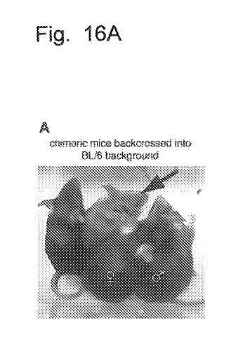

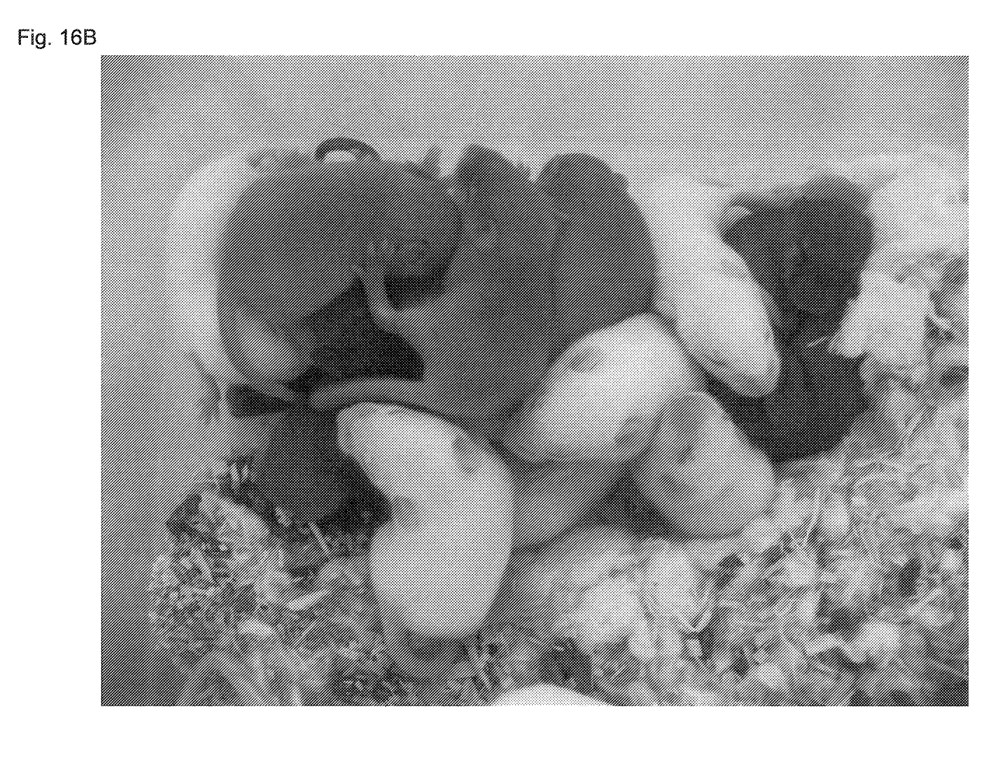

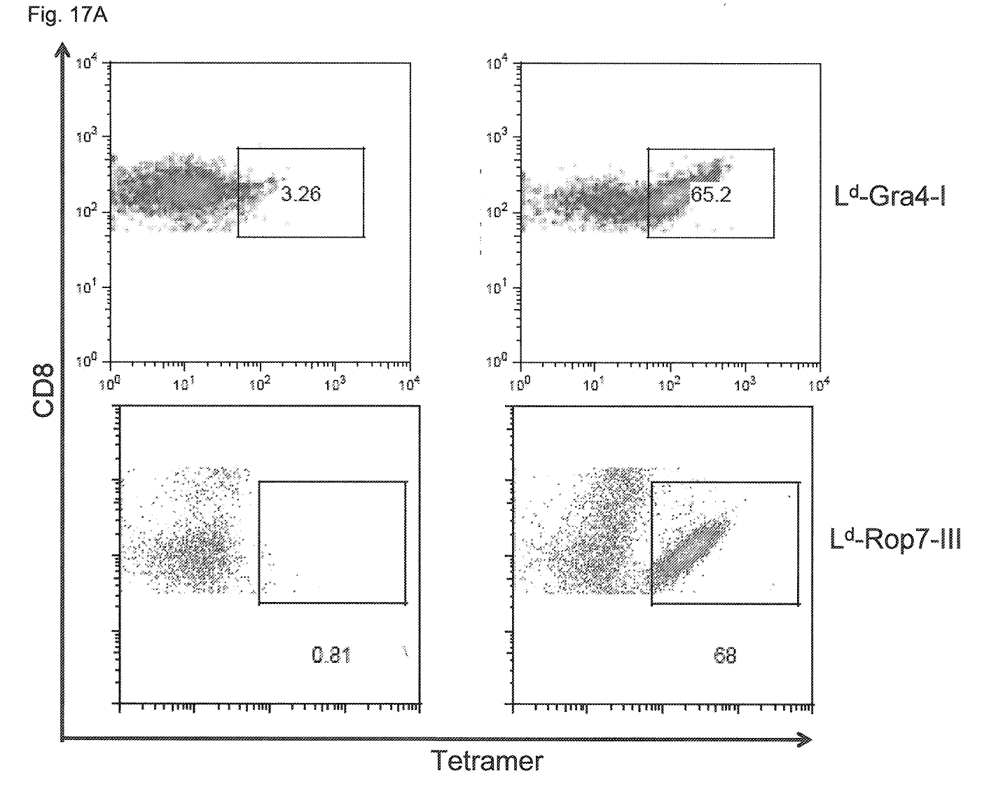

In order to test for germline transmission and to establish TN mouse lines, chimeric mice with H-2^{b} haplotype (K^{b}-Tg-tgd057⁵⁹⁻⁶⁶) were backcrossed into BL/6 background. Offspring with agouti coat color indicated germline-transmitting mice. Chimeric mice with H-2^{d} haplotype (L^{d}-Tg-Gra4¹⁰⁷⁻¹¹⁵ and L^{d}-Tg-Rop7¹⁶¹⁻¹⁶⁹) were backcrossed into Balb/c background and white offspring indicated germline transmission. Four of the SCNT-derived ES cell lines contributed to the germ cell lineage. FIGS. 16A and 16B show offspring resulting from back-crossing chimeric mice. All the litters from transmitting males were analyzed either by PCR (FIG. 17B) or by flow cytometry (FIGS. 17A and 17D) to identify offspring carrying the corresponding alpha- and beta-chain (see FIG. 17C for a list of mice and breeding). Offspring carrying the pre-defined TCR specificity were obtained (FIG. 17). It will be appreciated that only 25% of the offspring would be expected to have both rearranged TCR α and β chains derived from the original T cell.

We were able to secure germline transmission of the respective TCRs in five out of the five SCNT-ES cell lines derived from B6CF1 background, which should facilitate further in-depth characterization of these transnuclear CD8⁺ T cells. The resulting animals thus represent 3 different T cell receptor specificities (K^{b}-Tg-tgd057⁵⁹⁻⁶⁶, L^{d}-Tg-Gra4¹⁰⁷⁻¹¹⁵, and L^{d}-Tg-Rop7¹⁶¹⁻¹⁶⁹), restricted by two different MHC-I alleles (H-2L^{d} and H-2K^{b}). It was found that even among mice whose T cells all contain the correct, i.e., rearranged, TCR α and β chains, less than 100% of the T cells exhibit specificity for the epitope of interest in these experiments.

### Example 4

### Identification of Genomic TCR Rearrangements in Transnuclear Mice

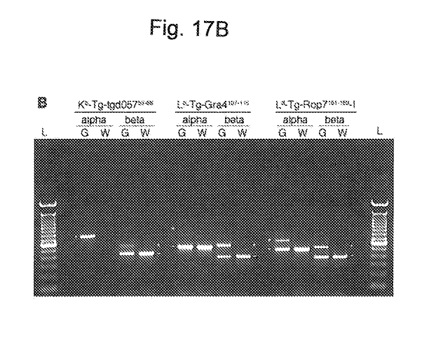

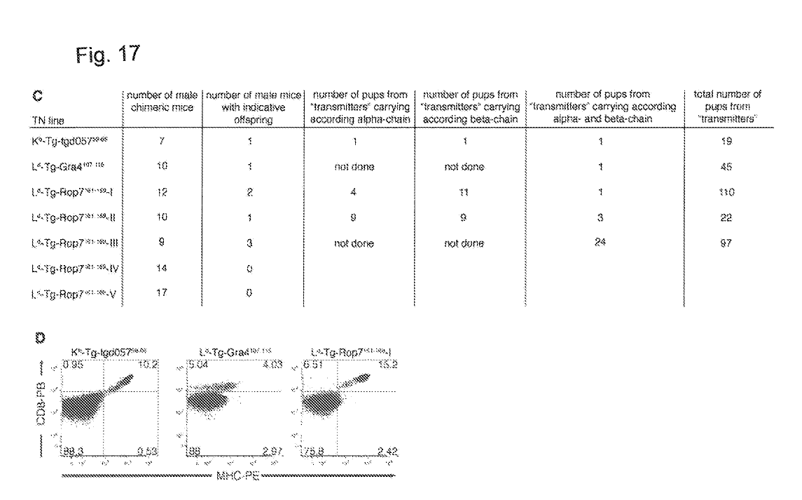

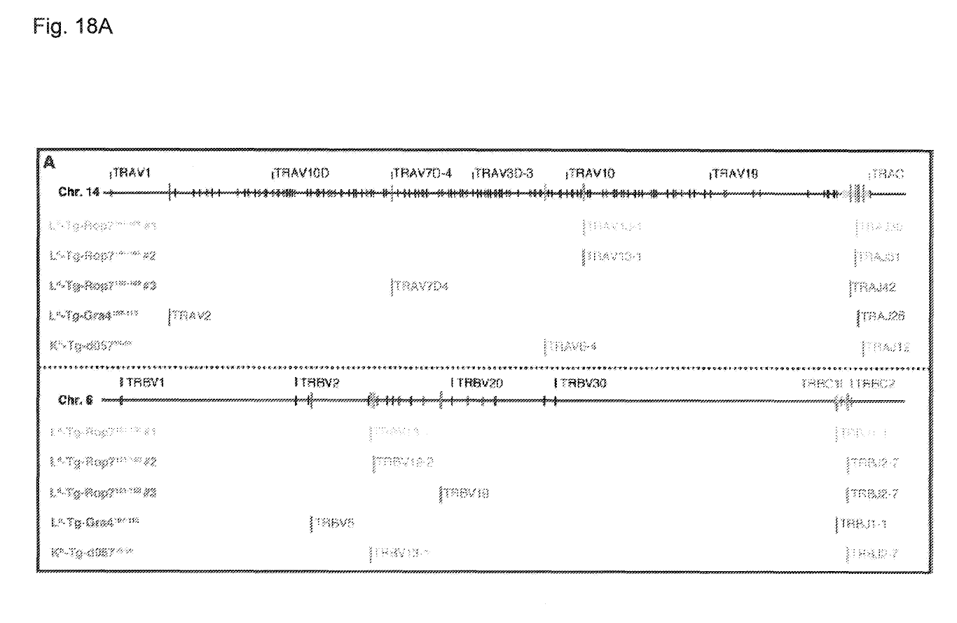

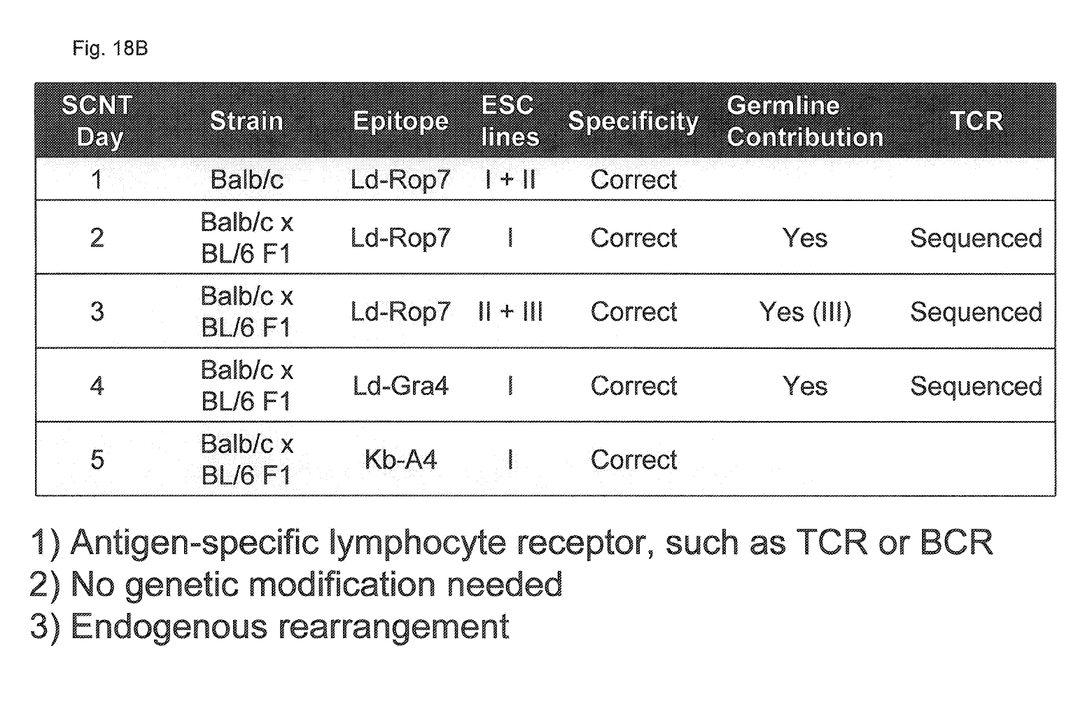

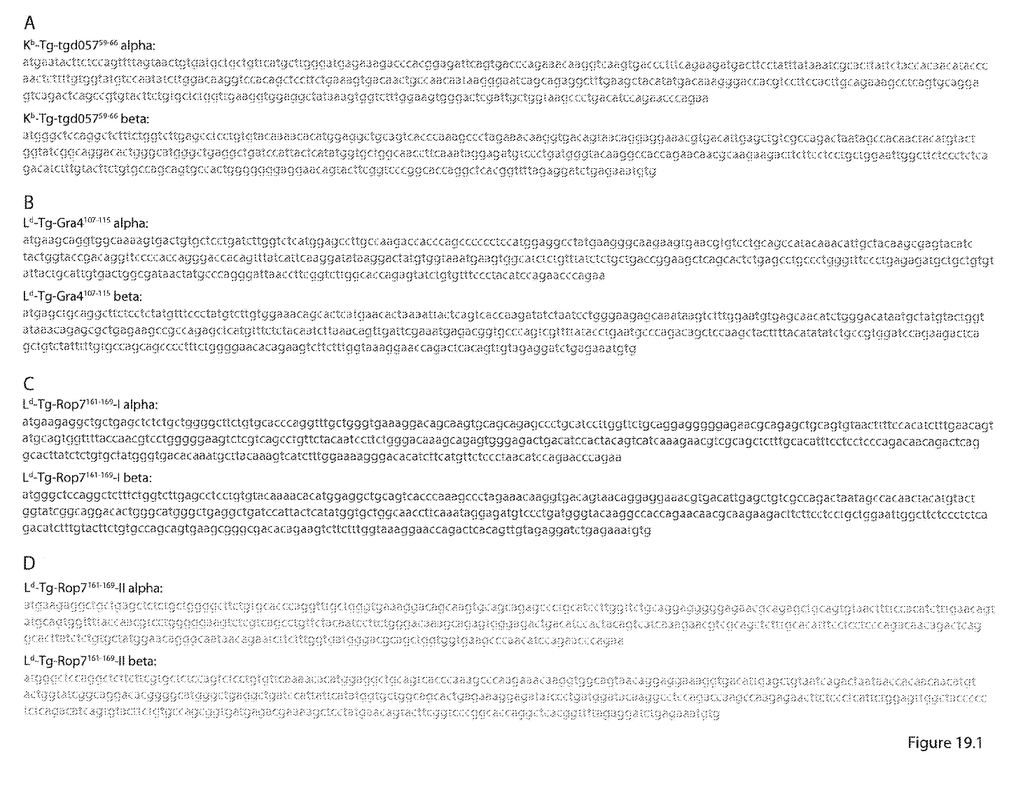

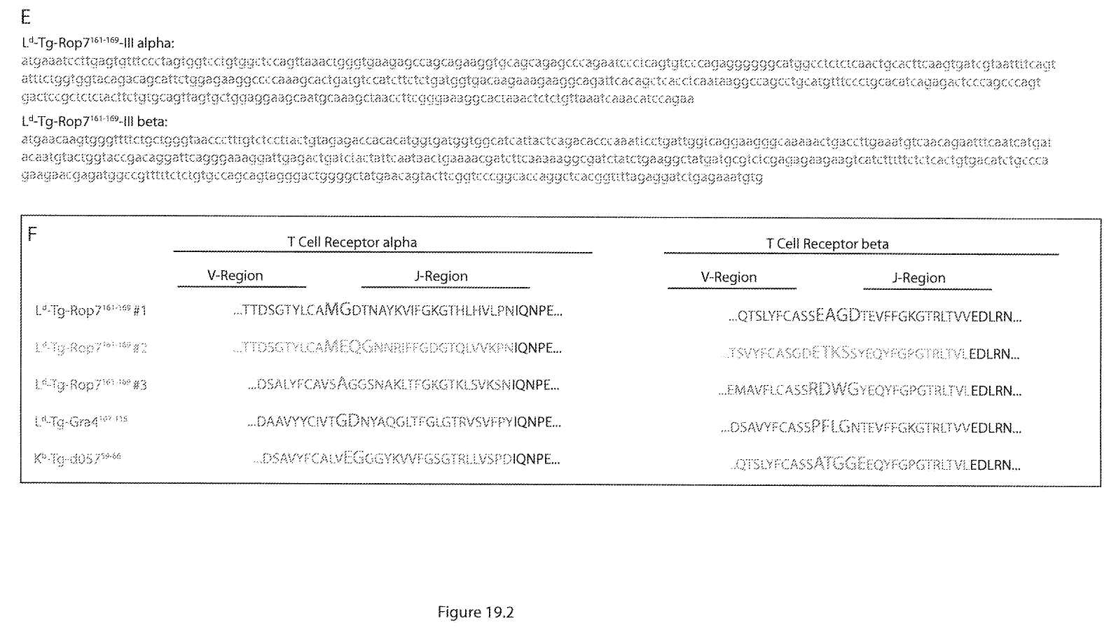

We identified the genomic rearrangements underlying the five CD8⁺ T cells on the B6CF1 background (FIG. 18A). Transnuclear CD8+ T cells from chimeric mice were FACsorted, RNA was isolated (Qiagen), and 5′-RACE was performed according to manufacturer's protocol (Invitrogen) using reported primers (28). We observed productive rearrangements for Vα6-4 and Jα12, and for Vβ13-1, Dβ2 and Jβ2-7 to generate the T cell receptor specific for K^{b}-Tg-tgd057⁵⁹⁻⁶⁶. We determined the rearrangements that generate the TCR specific for L^{d}-Tg-Gra4¹⁰⁷⁻¹¹⁵ as Vα2 and Jα26, and Vβ5, Dβ1, and Jβ1-1. We also identified the TCR for the three clones, which are specific for L^{d}-Tg-Rop7¹⁶¹⁻¹⁶⁹ as a recombination of Vα13-1 and Jα30, and Vβ13-1, Dβ1, and Jβ1-1 in clone 1, Vα13-1 and Jα31, and Vβ13-2, Dβ2, and Jβ2-7 in clone 2, and Vα7D4 and Jα42, and Vβ19, Dβ2, and Jβ2-7 in clone 3 (FIG. 19A-E for DNA sequence). A comparison of the peptide sequence revealed no obvious pattern of the complementary determining region 3 (CDR3) in the TCRs specific for the same MHC-peptide combination (FIG. 19F). FIG. 18B summarizes the data from a number of experiments.

### Example 5

### Further Characterization of Transnuclear Mice

Methods

Tetramer dissociation assay. 2×10⁶ negatively selected CD8⁺ T cells (BD) were stained with CD8-FITC (BD), live/dead blue (Invitrogen) and excess of tetramers for 45 min at 4° C., washed twice, resuspended in 1 ml PBS/2% FBS and incubated at 15° C. 100 μl aliquots were fixed immediately at various time points.

H-2L^{d} stabilization assay. 2×10⁵ T2-L^{d} cells were incubated for 3 h at 37° C. with the denoted range of peptides as described elsewhere (29). Peptide-stabilized surface L^{d} molecules were detected by staining with H-2Ld antibody (ABR), followed by staining with anti-mouse IgG2a/2b-FITC (BD).

Results

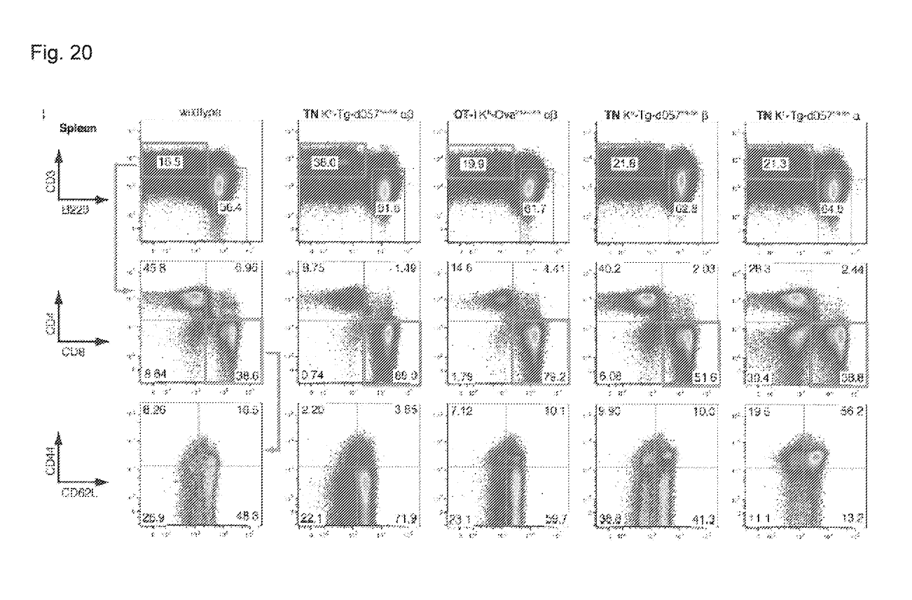

In order to characterize transnuclear mice, we compared splenocytes from the K^{b}-Tg-tgd057⁵⁹⁻⁶⁶ TN line with a transgenic line restricted to the same H-2K^{b}, OT-I. The OT-I line is specific for an ovalbumin-derived peptide (SIINFEKL) and since its generation, it has been used for a wide variety of immunological studies (19). A comparison of T cells (CD3⁺) and B cells (B220⁺) showed that the TN line has a relative increase in the CD3⁺ population (35.6% for TN versus 12.8% for wildtype) and that this population mainly consists of CD8⁺ T cells (91.3% for TN versus 43.1% for wildtype, FIG. 20. A skewing in the CD8⁺ population was also observed in the OT-I line (84.8%), but without a relative increase in CD3⁺ cells (17.7%). The expression level of CD44 and CD62L on CD8⁺ cells indicated that the majority of TN cells were CD44⁻CD62L⁺, which resembles a naive phenotype (64.1% for TN versus 69.6% for wildtype). To test whether a different H-2^{d} restricted transnuclear mouse can reproduce these results, we also compared the TN line specific for Gra4¹⁰⁷⁻¹¹⁵ to the transgenic line 2C. The 2C line was derived from an alloreactive clone (H-2^{b} cells anti-H-2^{d}) and its response against the QL9 peptide is particularly well characterized (20-22). Similarly, a relative increase in the CD3+ population was observed (49.2% for TN versus 27.3 for wildtype), with the majority being CD8+ (74.9% for TN versus 35.8% for wildtype) and naive (86% for TN versus 61.2% for wildtype).

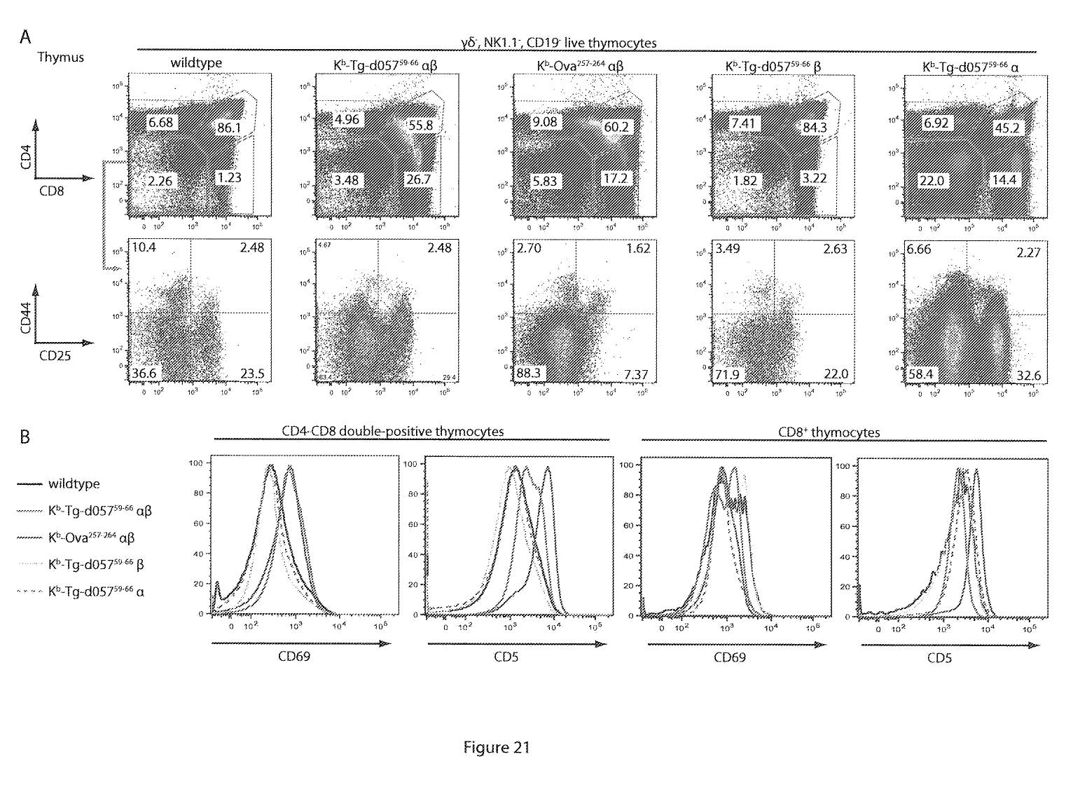

We next compared the thymic development of our TN line specific for K^{b}-Tg-tgd057⁵⁹⁻⁶⁶ to that of the transgenic line OT-I. An analysis of thymocytes using the markers CD4 and CD8 showed that TN mice have a decrease in the CD4-CD8 double-positive (DP) population (55.8% for TN versus 86.1% for wildtype) and a relative increase in the CD8 single-positive (SP) population (26.7% for TN versus 1.23% for wildtype, FIG. 21A, upper row). Similar to the TN line, the transgenic OT-I also showed a relative increase in the CD8 SP population (17.2%), and a decrease in the CD4-CD8 DP population (60.2%). A look at the early stages of T cell development revealed, that CD4-CD8 double-negative (DN) cells, have a similar developmental pattern (DN1-DN4) compared to wildtype mice, with slight reduction in the DN1 stage (10.4% for wildtype versus 4.67% for TN) and an increase in DN3 (23.5% for wildtype and 29.4% for TN) (FIG. 20, lower row). Contrary to the TN line, the transgenic OT-I line had a very different pattern than wildtype mice, with decreased DN3 stage (7.37%) and increased DN4 (88.3%). A look at CD5, a negative regulator of TCR signaling, showed that both K^{b}-Tg-tgd057⁵⁹⁻⁶⁶ and OT-I have a higher expression level than wildtype during the CD4-CD8 double-positive stage, with the TN line expressing less than the transgenic line (FIG. 2113). A difference of CD5-expression between transnuclear and transgenic can also be observed in the CD8 SP population. Upregulation of CD69 during the CD4-CD8 double-positive stage in the transnuclear as well as the transgenic line indicates that both are undergoing selection to a higher degree than wildtype mice.

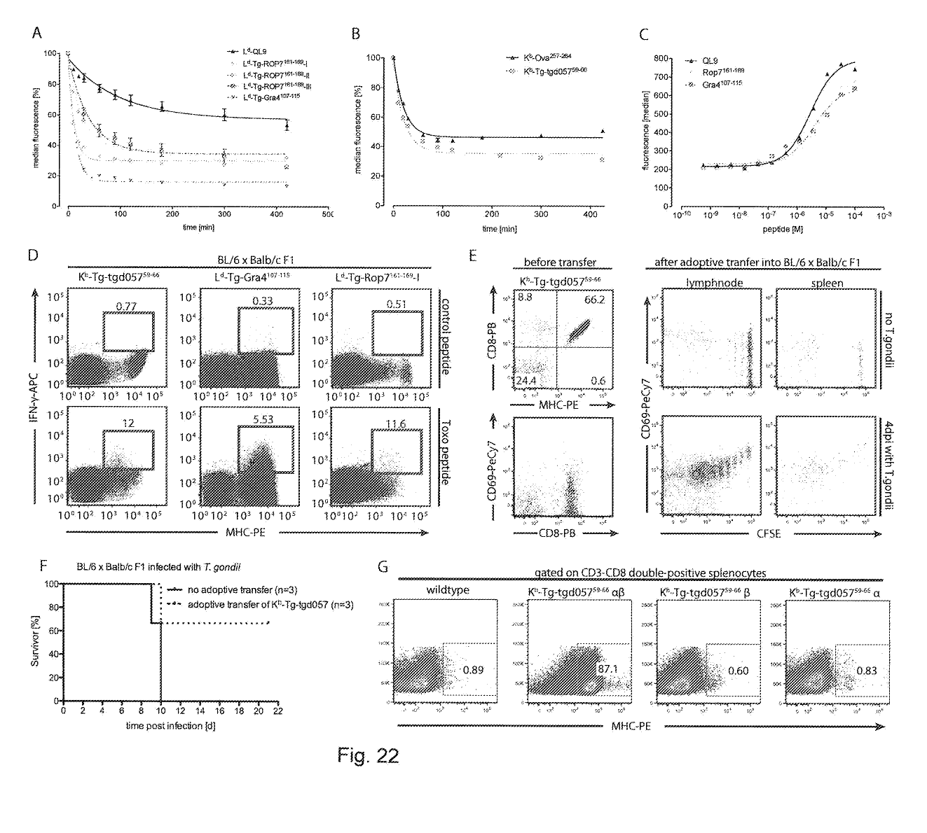

We hypothesized that TN CD8⁺ T cells, obtained via nuclear transfer of freshly isolated CD8⁺ T cells, without the use of in vitro culture and subsequent antigen-stimulation, may have MHC binding characteristics distinct from conventional TCR transgenic mice. We performed an MHC-I-tetramer dissociation assay, which showed that all tested TN CD8⁺ T cells of H-2L^{d} haplotype dissociated faster from their cognate peptide-MHC-I complex than transgenic 2C cells (FIG. 22A. Although we are aware of the limitations of tetramer dissociation as a surrogate parameter for TCR affinity (23), we consider it possible that the average T. gondii-specific H-2L^{d} restricted TCR may well be of lower affinity than the highly selected 2C receptor. The modest affinities of such CD8⁺ T cells might explain the massive expansion of CD8⁺ T cells commonly observed in viral infections. A comparison of transnuclear K^{b}-Tg-tgd057⁵⁹⁻⁶⁶ with transgenic K^{b}-Ova²⁵⁷⁻²⁶⁴ OT-I cells showed similar dissociation rates (FIG. 22B). The observed differences in dissociation rate of the H-2L^{d} T cells are not due to differences in peptide-MHC interactions, since the peptides QL9, Gra4¹⁰⁷⁻¹¹⁵, and Rop7¹⁶¹⁻¹⁶⁹ stabilized H-2L^{d} in TAP−/− cells equally well (FIG. 22C).

### Example 6

### Functional Activity of Transnuclear CD8⁺ T Cells in vitro and in vivo

Methods

Interferon-γ assay. Bone-marrow derived APC were loaded with 50 μM T. gondii peptide and incubated with CD8⁺ T cells at a 1:5 ratio plus 10 ng/ml IL-2 for 6d. Cultures were restimulated with freshly isolated splenocytes loaded with 1 μM T. gondii or control peptide at a 1:1 ratio for 6 h and 10 μg/ml brefeldin A was added during last 2 h as described previously (15).

Activation, proliferation and survival assay. Negatively selected CD8⁺ T cells were isolated from transnuclear mice, stained with CFSE according to manufacturer's protocol (Invitrogen), and injected i.v. into B6CF1 mice infected with 1×10⁶ Pru tachyzoites. Spleen and lymphnodes were isolated 4dpi, and analyzed by flow cytometry using CFSE, CD69-PE-Cy7, CD8-Pacificblue (all antibodies BD), live/dead blue (Invitrogen) and MHC-I tetramer-PE.

Results

To test whether transnuclear CD8⁺ T cells retain their function in vitro, we examined their ability to produce interferon-γ (IFN-γ) upon stimulation with peptide-loaded antigen-presenting cells. CD8⁺ T cells specific for K^{b}-Tg-tgd057⁵⁹⁻⁶⁶, L^{d}-Tg-Gra4¹⁰⁷⁻¹¹⁵, and L^{d}-Tg-Rop7¹⁶¹⁻¹⁶⁹ secrete IFN-γ only when stimulated with the corresponding peptide (FIG. 22D).

We next analyzed the ability of our transnuclear CD8⁺ T cells to respond in vivo to an infection with T. gondii (FIG. 22E). FACS analysis showed that 66% of CD8⁺ T cells from a chimeric mouse specific for K^{b}-Tg-tgd057⁵⁹⁻⁶⁶ stained with the K^{b}-Tg-tgd057⁵⁹⁻⁶⁶ MHC-I tetramer after purification (upper left plot). These cells had a resting phenotype as judged by the absence of CD69 (lower left plot) prior to adoptive transfer. We transferred 1.2×10⁶ CFSE-labeled cells per mouse (about 8×10⁵ tetramer-positive CD8⁺ T cells) and observed upregulation of CD69 and robust proliferation upon subsequent infection of the recipients with T. gondii. When challenged with a lethal dose of T. gondii, 2 out of 3 mice survived the acute phase of infection (FIG. 22F), whereas all of the controls died.

### Example 7

### Further Studies Using Transnuclear Mice

To test how efficiently an endogenous pre-rearranged α- or β-chain, which is part of a specific TCR, can lead to a TCR with the same specificity, we analyzed mice carrying only one copy of either the α- or the β-chain of K^{b}-Tg-tgd057⁵⁹⁻⁶⁶. We determined the presence of T cell receptors specific for K^{b}-Tg-tgd057⁵⁹⁻⁶⁶ on CD3⁺ CD8⁺ T cells carrying a single copy of the β-chain (FIG. 22G). Compared to wildtype mice, there was no increase in specific T cells (0.60% for β-chain versus 0.89% for wildtype). Similarly, the presence of one copy of α-chain did not increase the presence of according specific T cells (0.83%).

### Example 8

### Generation and Characterization of Transnuclear Mice with Pre-Defined Specificity via Somatic Cell Nuclear Transfer Using B Cells as Nuclear Donors

SCNT was used to generate transnuclear mice using B cells specific for an epitope of an antigen of interest (Ovalbumin) as nuclear donors. To isolate B cells specific for a particular epitope, B6CF1 male mice were injected with Ovalbumin and complete Freund's adjuvant (CFA) intraperitoneally. Mice were then boosted with Ovalbumin and incomplete Freund's adjuvant once a week for a minimum of two consecutive weeks. Successful immunization was confirmed by analyzing the peripheral blood for the presence of Ovalbumin-specific antibodies (using ELISA). To isolate B cells, the spleen of an immunized mouse was disrupted between two frosted glass slides. The erythrocytes were lysed with ammonium chloride and the remaining cells washed two times with PBS and passed through a 70 μm cell strainer. Subsequently, the cells were incubated with saturating amounts of Ovalbumin-PE complexes comprising multiple Ovalbumin molecules cross-linked together (“Ova complex”), FITC-conjugated anti-IgM antibody, and anti-CD19-allophycocyanin (CD19-APC) antibody. Cells were sorted using a FACSAria flow cytometer and the following criteria: CD19-positive, IgM-negative, and Ovalbumin-positive. It is also possible to sort directly for a certain isotype, such as IgG1, or any other isotype, e.g., using a labeled antibody that binds to IgG1 constant region.

Sorted cells were kept in RPMI 1640 medium supplemented with 10% fetal calf serum, penicillin (100 U/ml), streptomycin (100 μg/ml) and 2 mM glutamine, and used for nuclear transfer within three hours after sorting. SCNT was performed as described above in order to derive ES cell lines. The recipient oocyte was Bl/6 DBA2 F2 (BDF2). Three SCNT-ES cell lines were derived of which one was specific for Ovalbumin. The Ova-specific SCNT-derived ES cell line was used to generate chimeras by injection into blastocysts, which were transferred to pseudopregnant female mice. Resulting chimeras were back-crossed into the BL/6 background, resulting in a TN IgG1-Ova line.

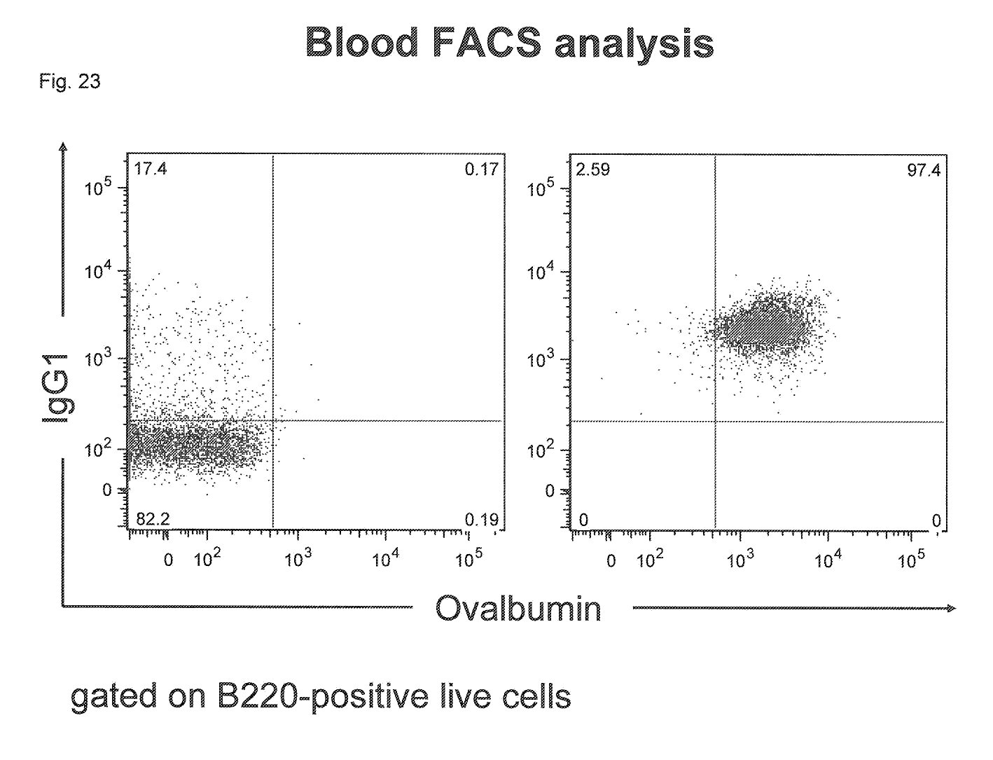

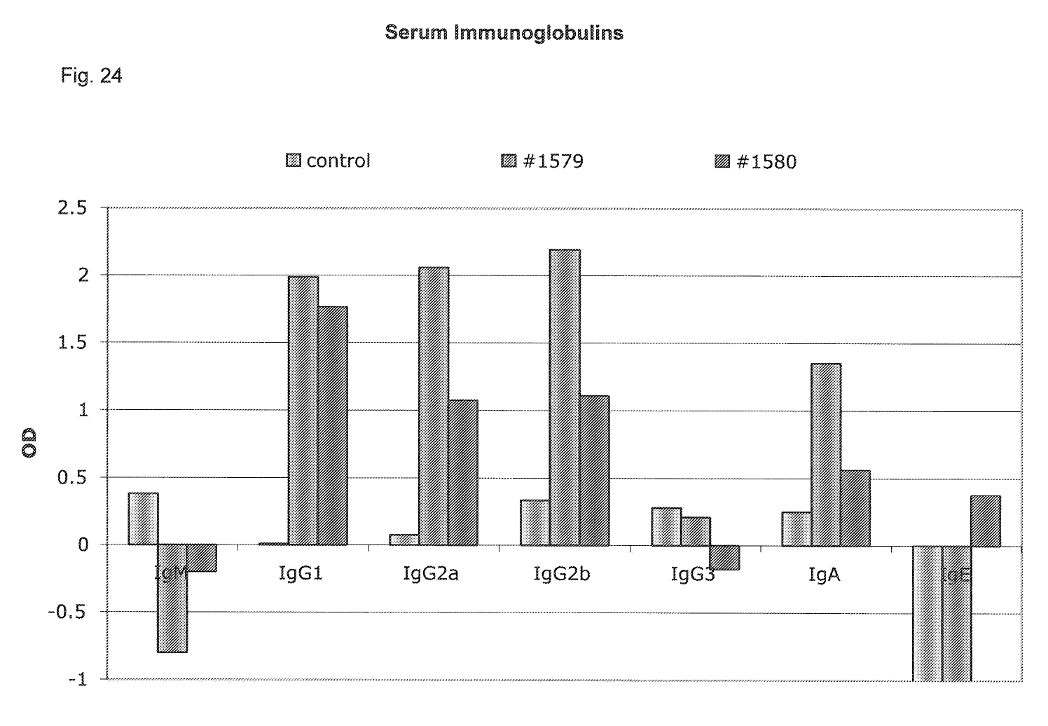

Flow cytometry was performed to analyze blood cells obtained from a TN IgG1-Ova mouse. Cells were stained using labeled anti-IgG1 antibody and labeled Ova complex. Results for B220 positive cells are shown in FIG. 23. A control mouse (left) has 17.4% IgG1⁺ B cells and almost no specificity for Ovalbumin (0.17% and 0.19%) while a TN IgG1-Ova mouse has almost exclusively IgG1⁺ B cells (2.59%+97.4%) with the great majority being specific for Ovalbumin (97.4%). ELISA analysis of serum immunoglobulins from transnuclear mice derived from B cells expressing Ovalbumin-specific IgG1 was performed. Plates were coated with Ovalbumin and then incubated with serum from either control or two different mice (#1579 and #1580) from the TN IgG1-Ova line. As shown in FIG. 24, control mice have very few immunoglobulins specific for Ovalbumin, independent of the isotype while transnuclear mice exhibit considerable levels of Ovalbumin-specific immunoglobulins. The Ova-specific immunoglobulins were of the IgG1, IgG2a, IgG2b, IgA, and possibly IgE isotype, consistent with removal of the IgM and IgG3 loci from the genome once a B cell has switched to IgG1.

### Example 9

### Generation of Monoclonal Mice with Pre-Defined Specificity Against C. trachomatis Epitopes via SCNT

Example 3 is repeated using T cells that are specific for C. trachomatis epitopes identified as described in Example 1, except that tetraploid complementation is used to generate chimeric mice that are derived primarily from ES cells.

### Example 10

### Generation of Transnuclear Mice Using T Cells Obtained from Mice Immunized with Candidate Mycobacterium tuberculosis Vaccine

Mice are immunized with DNA and/or recombinant modified vaccinia virus Ankara strain (MVA) vaccines, each encoding Mycobacterium tuberculosis antigen 85A fused to the tissue plasminogen activator leader sequence (30). CD4⁺ and CD8⁺ T cells specific for antigen 85 are identified and used to generate transnuclear mice by SCNT. In some experiments, T cells specific for each of the following antigen 85 epitopes are used:

| | | |
|---|---|---|
| p11b | EWYDQSGLSVVMPVGGQSSF | (SEQ ID NO: 7) |
| p15c | TFLTSELPGWLQANRHVKPT | (SEQ ID NO: 8) |
| p24c | QRNDPLLNVGKLIANNTRVW | (SEQ ID NO: 9) |
| p27c | LGGNNLPAKFLEGFVRTSNI | (SEQ ID NO: 10) |

Mice having T cells specific for antigen 85 are identified. T cells are isolated from these mice and their functional activity is assessed in a manner similar to that described in Example 6. In other experiment, transnuclear mice having T cells specific for antigen 85 are challenged with 10⁶ CFU M. tuberculosis bacteria. The ability of these mice to resist infection in the lungs and spleen relative to controls is assessed.

### Example 11

### Generation of Transnuclear Mice Using B Cells Obtained from Mice Immunized with HIV-1 gp120 Core Protein

Recombinant HIV-1 gp120 is prepared from CHO cells as described (31) and used to immunize mice. B cells specific for gp120 epitopes are isolated. In some experiments the B cells are selected to be IgG1, IgM, or IgA positive. The isolated B cells are used to generate transnuclear mice by SCNT. The antibody response to inoculation with gp120 is assessed.

### Example 12

Example 11 is repeated except that B cells specific for gp120 are reprogrammed to iPS cells as described in Hanna, et al., Cell, 133(2):250-64 (2008). The iPS cells are then used to generate mice.

### References for Examples 3-12

1. P. Wong, E. G. Pamer, Annu. Rev. Immunol. 21, 29 (2003).
2. I. J. Amanna, M. K. Slifka, S. Crotty, Immunol. Rev. 211, 320 (2006).
3. L. E. Harrington, R. Most Rv, J. L. Whitton, R. Ahmed, J. Virol. 76, 3329 (2002).
4. S. Gredmark-Russ, E. J. Cheung, M. K. Isaacson, H. L. Ploegh, G. M. Grotenbreg, J. Virol. (2008).
5. M. Moutaftsi et al., Nat. Biotechnol. 24, 817 (2006).
6. T. Wakayama, R. Yanagimachi, Mol. Reprod. Dev. 58, 376 (2001).
7. S. Kishigami et al., Nat Protoc 1, 125 (2006).
8. K. Eggan et al., Proc. Natl. Acad. Sci. USA 98, 6209 (2001).
9. K. Inoue et al., Curr. Biol. 15, 1114 (2005).
10. K. Hochedlinger, R. Jaenisch, Nature 415, 1035 (2002).
11. T. Serwold, K. Hochedlinger, M. A. Inlay, R. Jaenisch, I. L. Weissman, J. Immunol. 179, 928 (2007).
12. S. B. Koralov, T. I. Novobrantseva, K. Hochedlinger, R. Jaenisch, K. Rajewsky, J. Exp. Med. 201, 341 (2005).
13. Y. Suzuki, J. S. Remington, J. Immunol. 144, 1954 (1990).
14. F. S. Dzierszinski, C. A. Hunter, Parasite Immunol. 30, 235 (2008).
15. E. M. Frickel et al., J. Infect. Dis. (2008).
16. N. Blanchard et al., Nat. Immunol. 9, 937 (2008).
17. S. Kishigami et al., J Reprod Dev 53, 165 (2007).
18. S. Kishigami et al., Biochem. Biophys. Res. Commun. 340, 183 (2006).
19. K. A. Hogquist et al., Cell 76, 17 (1994).
20. D. M. Kranz, D. H. Sherman, M. V. Sitkovsky, M. S. Pasternack, H. N. Eisen, Proc. Natl. Acad. Sci. USA 81, 573 (1984).
21. W. C. Sha et al., Nature 336, 73 (1988).
22. J. Chen, H. N. Eisen, D. M. Kranz, Microbes Infect 5, 233 (2003).
23. X. L. Wang, J. D. Altman, J. Immunol. Methods 280, 25 (2003).
24. V. Kouskoff, K. Signorelli, C. Benoist, D. Mathis, J. Immunol. Methods 180, 273 (1995).
25. H. Pircher, K. Burki, R. Lang, H. Hengartner, R. M. Zinkernagel, Nature 342, 559 (1989).
26. C. Mamalaki et al., Dev Immunol 3, 159 (1993).
27. M. J. Barnden, J. Allison, W. R. Heath, F. R. Carbone, Immunol. Cell Biol. 76, 34 (1998).
28. W. W. Overwijk et al., J. Exp. Med. 198, 569 (2003).
29. C. J. Schlueter, T. C. Manning, B. A. Schodin, D. M. Kranz, J. Immunol. 157, 4478 (1996).
30. McShane, H., et al. Infection and Immunity, 70(3): 1623-1626 (2002).
31. Li, H., et al. J Immunol., 180(6):4011-21, (2008).

One skilled in the art readily appreciates that the present invention is well adapted to carry out the objects and obtain the ends and advantages mentioned, as well as those inherent therein. The details of the description and the examples herein are representative of certain embodiments, are exemplary, and are not intended as limitations on the scope of the invention. Modifications therein and other uses will occur to those skilled in the art. These modifications are encompassed within the spirit of the invention. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth in so many words herein. It should also be understood that any embodiment or aspect of the invention can be explicitly excluded from the claims, regardless of whether the specific exclusion is recited in the specification.

Where the claims or description relate to a composition of matter, e.g., an animal or cell, it is to be understood that methods of making or using the composition of matter according to any of the methods disclosed herein, and methods of using the composition of matter for any of the purposes disclosed herein are aspects of the invention, unless otherwise indicated or unless it would be evident to one of ordinary skill in the art that a contradiction or inconsistency would arise. Where the claims or description relate to a method, e.g., a method of making a non-human animal and/or cell, it is to be understood that the non-human animal and/or cell, and methods of using it, are aspects of the invention, unless otherwise indicated or unless it would be evident to one of ordinary skill in the art that a contradiction or inconsistency would arise.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” generally includes numbers that fall within a range of 1% or in some embodiments within a range of 5% of a number or in some embodiments within a range of 10% of a number in either direction (greater than or less than the number) unless otherwise stated or otherwise evident from the context (except where such number would impermissibly exceed 100% of a possible value). It should be understood that, unless clearly indicated to the contrary, in any methods claimed herein that include more than one act, the order of the acts of the method is not necessarily limited to the order in which the acts of the method are recited, but the invention includes embodiments in which the order is so limited. It should also be understood that any product or composition of the invention may be “isolated”, e.g., separated from at least some of the components with which it is usually associated in nature; prepared or purified by a process that involves the hand of man; and/or not occurring in nature.

1. A method of producing a non-human mammal, the method comprising:
(a) providing a non-human mammalian T or B cell that has a predefined specificity, wherein the mammalian T or B cell is not a natural killer (NK) T cell; and
(b) generating a non-human mammal using the mammalian T or B cell, wherein at least some cells of the non-human mammal contain TCR or BCR genes derived from the mammalian T or B cell.

2. The method of claim 1, wherein the mammalian T or B cell has rearranged TCR alpha and beta chain genes or rearranged BCR heavy and light chain genes, respectively, and at least some cells of the non-human mammal contain rearranged TCR alpha and beta chain genes or BCR heavy and light chain genes, respectively, that assemble to form a TCR or BCR with the same specificity as those of the T or B cell.

3. The method of claim 1, wherein the cell is a conventional T cell.

4. The method of claim 1, wherein the cell is a CD8+ T cell.

5. The method of claim 1, wherein the T or B cell is specific for a predefined epitope.

6. The method of claim 5, wherein the predefined epitope is a peptide.

7. The method of claim 1, wherein the T or B cell is specific for a predefined antigen.

8. The method of claim 7, wherein the predefined antigen is a protein.

9. The method of claim 7, wherein the predefined antigen is produced by a microorganism.

10. The method of claim 7, wherein the predefined antigen is produced by a pathogen.

11. The method of claim 7, wherein the predefined antigen is a tumor antigen.

12. The method of claim 1, wherein the non-human mammal is a mouse.

13. The method of claim 1, wherein the T cell has a non-invariant TCR alpha chain.

14. The method of claim 1, wherein generating the non-human mammal comprises reprogramming the nucleus of the T or B cell to pluripotency.

15. The method of claim 1, wherein generating the non-human mammal comprises performing somatic cell nuclear transfer (SCNT) using a T or B cell with a predefined specificity as a nuclear donor.

16. The method of claim 15, wherein SCNT is performed within 24 hours of isolating the T or B cell from an animal.

17. The method of claim 15, wherein the SCNT embryo is cultured in medium containing an inhibitor of histone deacetylase.

18. The method of claim 17, wherein the inhibitor is trichostatin A.

19. The method of claim 1, wherein generating the non-human mammal comprises performing two step cloning.

20. The method of claim 19, wherein said two step cloning comprises introducing ES cells into mouse tetraploid blastocysts by injection under conditions that result in production of an embryo.

21. The method of claim 1, wherein the non-human mammal is not genetically modified.

22. The method of claim 1, wherein T and B cells of the non-human mammal do not contain a TCR or BCR transgene.

23. The method of claim 1, wherein the method comprises:
(a) reprogramming a T or B cell that has a predefined specificity of interest to form an induced pluripotent stem (iPS) cell; and
(b) generating a non-human mammal from the iPS cell.

24. The method of claim 1, wherein the method comprises:
(a) isolating from a first non-human mammal a T or B cell that has a predefined specificity of interest; and
(b) generating a second non-human mammal from the T or B cell.

25. The method of claim 24, wherein the method comprises immunizing the first non-human mammal with an antigen of interest prior to isolating the T or B cell.

26. The method of claim 24, wherein the method comprises infecting the first non-human mammal with a microorganism of interest prior to isolating the T or B cell.

27. The method of claim 24, wherein the step of isolating comprises:
(a) obtaining T cells from the first non-human mammal;
(b) contacting the T cells with an MHC-epitope complex; and
(c) isolating a T cell that binds to the MHC-epitope complex.

28. The method of claim 24, wherein the step of isolating comprises:
(a) obtaining B cells from the first non-human mammal;
(b) contacting the B cells with an epitope or antigen; and
(c) isolating a B cell that binds to the epitope or antigen.

29. The method of claim 24, wherein the step of isolating comprises:
(a) obtaining B cells from the first non-human mammal;
(b) culturing individual B cells under conditions in which antibody is secreted; and
(c) isolating a B cell that secretes an antibody having the predefined specificity.

30. The method of claim 1, further comprising isolating T or B cells from the non-human mammal.

31. The method of claim 30, further comprising analyzing the T or B cells.

32. The method of claim 1, further comprising analyzing the immune response of the non- human mammal to an antigen towards which the T or B cell has specificity.

33. A non-human mammal produced according to the method of claim 1 or a descendant thereof.

34-35. (canceled)

36. An ES cell or iPS cell produced from a T or B cell with a predefined specificity.

37-40. (canceled)

41. A method of producing a non-human mammal, the method comprising
(a) providing a T or B cell isolated from an individual suffering from or at risk of a disease; and
(b) generating a non-human mammal from the T or B cell.

42-45. (canceled)

46. The method of claim 1, wherein at least 50% of the T cells or at least 50% of the B cells of the non-human mammal are specific for a predefined antigen or epitope, and wherein T and B cells of the non- human mammal do not comprise a TCR or BCR transgene, respectively.
